(19) United States
(12) Patent Application Publication
(10) Pub. No.: US 2021/0401834 A1
(43) Pub. Date: Dec. 30, 2021

(54) MODIFIED RELEASE FORMULATIONS OF MODIFIED FORMS OF TRIMETAZIDINE

(71) Applicant: IMBRIA PHARMACEUTICALS, INC., Boston, MA (US)

(72) Inventors: George Mooney, Boston, MA (US); Scott Herbig, Boston, MA (US)

(21) Appl. No.: 17/359,726

(22) Filed: Jun. 28, 2021

Related U.S. Application Data
(60) Provisional application No. 63/183,299, filed on May 3, 2021.
(60) Provisional application No. 63/046,117, filed on Jun. 30, 2020.

(51) Int. Cl. A61K 31/496; A61K 47/38; A61K 9/00

(52) U.S. Cl. CPC A61K 31/496; A61K 9/2054; A61K 9/0053; A61K 47/38

(57) ABSTRACT

The invention provides pharmaceutical compositions that contain modified release formulations of modified forms of trimetazidine, such as CV-8972. The compositions include an erodible polymer, such as hydroxypropyl methylcellulose (HPMC), that allows sustained release of the modified form of trimetazidine and it metabolic products in the body. The invention also provides methods of treating conditions, including angina and heart failure, using such compositions.

This application claims the benefit of, and priority to, U.S. Provisional Patent Application No. 63/046,117, filed Jun. 30, 2020, and U.S. Provisional Patent Application No. 63/183,299, filed May 3, 2021, the contents of each of which are incorporated by reference.

### FIELD OF THE INVENTION

The invention relates pharmaceutical compositions containing modified-release formulation of modified forms of trimetazidine and the use of such compositions to treat medical conditions, including angina and heart failure.

### BACKGROUND

Heart disease is the leading cause of death worldwide, accounting for 15 million deaths across the globe in 2015. In many forms of heart disease, decreased cardiac efficiency stems from changes in mitochondrial energy metabolism. Mitochondria are sub-cellular compartments in which metabolites derived from glucose and fatty acids are oxidized to produce high-energy molecules. Increasing fatty acid oxidation in the heart decreases glucose oxidation, and vice versa. Glucose oxidation is a more efficient source of energy, but in certain types of heart disease, such as angina, heart failure, ischemic heart disease, and diabetic cardiomyopathies, fatty acid oxidation predominates in cardiac mitochondria. As a result, the pumping capacity of the heart is reduced.

### SUMMARY

CV-8972 (U.S. Pat. No. 10,556,013, the content of which is incorporated by reference herein in its entirety), a modified form trimetazidine, was recently identified as a promising therapeutic candidate for cardiovascular conditions. CV-8972 has the IUPAC name 2-[4-[(2,3,4-trimethoxyphenyl)methyl]piperazin-1-yl]ethyl pyridine-3-carboxylate and the following structure:

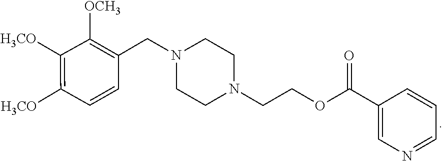 [SMILES: COc1ccc(CN2CCN(CCOC(=O)c3cccnc3)CC2)c(OC)c1OC]

Without being limited by any particular theory or mechanism of action, it is believed that CV-8972 is broken down sequentially into several specific, biologically active metabolites when CV-8972 is provided to humans. CV-8972 is initially broken down into niacin and a modified form of trimetazidine, which is identified also identified in U.S. Pat. No. 10,556,013 as CV-8814, having the following structure:

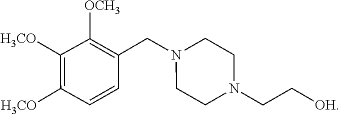 [SMILES: COc1ccc(CN2CCN(CCO)CC2)c(OC)c1OC]

CV-8814 is subsequently converted in the body to trimetazidine. Significantly, both trimetazidine and CV-8814 promote glucose oxidation by blocking 3-ketoacyl-CoA thiolase, and thus both are active pharmaceutical ingredients (APIs). Consequently, CV-8972 is metabolized in the body into individual components that exert distinct biochemical effects to promote glucose oxidation and improve overall mitochondrial respiration in the heart. Because CV-8972 yields different metabolic products that act synergistically, CV-8972 is useful as a therapeutic agent for treating heart diseases characterized by elevated fatty acid oxidation. The CV-8814 and trimetazidine produced from CV-8972 shift cardiac metabolism from fatty acid oxidation to glucose oxidation to allow the use of a more efficient source of energy. The niacin produced from CV-8972 stimulates metabolic pathways that are common to oxidation of both glucose and fatty acids and that may also be impaired in patients with heart disease.

This invention recognizes that CV-8972 is rapidly broken down in the body to niacin and CV-8814. The invention also recognizes that high amounts of niacin in the body can have certain side effects, such as flushing. The invention further recognizes that a formulation that slows and/or controls the breakdown of CV-8972 into niacin and CV-8814 would provide a beneficial effect of reducing or eliminating any niacin side effects (e.g., flushing) while also prolonging the efficacy of a single dose of CV-8972, thereby leading to less frequent dosing.

In that manner, the invention provides modified-release formulations of CV-8972 that promote gradual metabolism of CV-8972 in the digestive tract. The modified-release formulations of the invention have improved therapeutic properties because they lead to less acute and more prolonged increases of the pharmacologically active products of CV-8972 in circulation. Thus, the formulations provided herein greatly increase the utility of this promising new drug candidate.

The compositions of the invention contain a mixture that includes a modified form of trimetazidine, such as CV-8972, and an erodible polymer, such as hydroxypropyl methylcellulose (HPMC). When such compositions are administered orally to a subject, the mixture absorbs water in the digestive tract, and the polymer gradually breaks down. Consequently, maximum levels of the active pharmaceutical ingredients in the subject's plasma are achieved two or more hours after administration of the compositions, and peak levels are about 50% lower than those produced by conventional formulations containing the same dose of the therapeutic agent. The compositions of the invention thus provide extended periods in which the metabolic products of CV-8972 or other modified forms of trimetazidine are maintained in the body above a therapeutic threshold while mitigating side effects that result from high peak levels. The invention further provides methods of treating cardiac conditions by providing the compositions described herein.

The pharmaceutical compositions of the invention are useful for treating any condition that can be ameliorated by improving cardiac mitochondrial function. In particular, the compositions are useful for treating cardiovascular conditions, such as angina and heart failure. The compositions can be easily administered orally, e.g., as a tablet or capsule. Moreover due to the sustained release of therapeutic agent, the compositions need only be taken once or twice per day.

In an aspect, the invention provides pharmaceutical compositions containing a mixture that includes a modified form of trimetazidine and an erodible polymer that promotes swelling of the mixture in an aqueous environment.

The modified form of trimetazidine may be any compound that is structurally related to trimetazidine, has a similar biochemical function to trimetazidine, or is metabolized in the body to produce trimetazidine. The modified form of trimetazidine may have a structure of one of Formulas (IX) and (X):

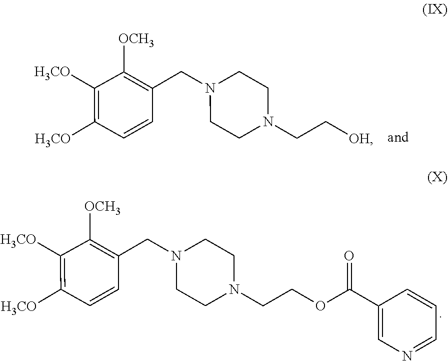 [SMILES: COc1ccc(CN2CCN(CCO)CC2)c(OC)c1OC ; COc1ccc(CN2CCN(CCOC(=O)c3cccnc3)CC2)c(OC)c1OC]

The erodible polymer may be any biocompatible polymer that breaks down in the body and promotes swelling of a mixture containing the modified form of trimetazidine. The polymer may be biodegradable. The polymer may be hydrophilic. The polymer may promote formation of a hydrogel. The polymer may be a cellulose derivative. The polymer may be methylcellulose, hydroxypropyl methylcellulose, ethyl cellulose, hydroxyethyl cellulose, or sodium carboxymethylcellulose.

The mixture may contain multiple polymeric forms of HPMC. The different polymeric forms of HPMC may differ in one or more properties. The different polymeric forms of HPMC may differ in one or more of viscosity, degree of methoxyl substitution, degree of hydroxypropoxyl substitution, and average molecule weight.

Each polymeric form of HPMC may independently have a defined viscosity. The viscosity may be from about 2 cP to about 4 cP, from about 4 cP to about 6 cP, from about 5 cP to about 8 cP, from about 12 cP to about 18 cP, from about 40 cP to about 60 cP, from about 80 cP to about 120 cP, from about 300 cP to about 500 cP, from about 1200 cP to about 2400 cP, from about 2500 cP to about 5000 cP, from about 9000 cP to about 18,000 cP, from about 12,000 cP to about 24,000 cP, from about 12,000 cP to about 24,000 cP, from about 75,000 cP to about 150,000 cP, at least about 2 cP at least about 4 cP at least about 5 cP at least about 12 cP at least about 40 cP at least about 80 cP at least about 300 cP at least about 1200 cP at least about 2500 cP at least about 9000 cP at least about 12,000 cP at least about 12,000 cP at least about 75,000 cP less than about 4 cP, less than about 6 cP, less than about 8 cP, less than about 18 cP, less than about 60 cP, less than about 120 cP, less than about 500 cP, less than about 2400 cP, less than about 5000 cP, less than about 18,000 cP, less than about 24,000 cP, less than about 24,000 cP, or less than about 150,000 cP for a 2% aqueous solution of the polymeric form at 20° C.

Each polymeric form of HPMC may independently have a defined degree of methoxyl substitution. The degree of methoxyl substitution may be from about 19% to about 24%, from about 22% to about 24%, from about 27% to about 30%, from about 27% to about 30%, or from about 28% to about 32%.

Each polymeric form of HPMC may independently have a defined degree of hydroxypropoxyl substitution. The degree of hydroxypropoxyl substitution may be from about 4% to about 8%, from about 7% to about 10%, from about 7% to about 12%, from about 8% to about 10%, from about 8% to about 11%, or from about 9% to about 12%.

Each polymeric form of HPMC may independently have a defined average molecular weight. The average molecular weight may be about 10 kDa, about 13 kDa, about 20 kDa, about 26 kDa, about 41 kDa, about 63 kDa, about 86 kDa, about 110 kDa, about 120 kDa, about 140 kDa, about 180 kDa, or about 220 kDa.

Mixtures containing multiple polymeric forms of HPMC may contain one polymeric form at a defined amount. The HPMC may contain about 50%, about 60%, about 70%, about 80%, about 90%, about 95%, about 96%, about 97%, about 98%, about 99%, at least 50%, at least 60%, at least 70%, at least 80%, at least 90%, at least 95%, at least 96%, at least 97%, at least 98%, or at least 99% by weight of one polymeric form.

The mixture may contain a defined amount of the modified form of trimetazidine. The mixture may contain at least 5%, at least 10%, at least 20%, at least 30%, at least 40%, at least 50%, at least 60%, at least 70%, or at least 80% by weight of the modified form of trimetazidine.

The pharmaceutical composition may be formulated for a particular route of administration. The pharmaceutical may be formulated for oral, enteral, intravenous, or rectal administration.

The pharmaceutical composition may be formulated as a unit dosage containing a defined amount of the modified form of trimetazidine. The unit dosage may contain about 5 mg, about 10 mg, about 20 mg, about 50 mg, about 100 mg, about 200 mg, about 500 mg, from about 5 mg to about 10 mg, from about 5 mg to about 20 mg, from about 5 mg to about 50 mg, from about 5 mg to about 100 mg, from about 5 mg to about 200 mg, from about 5 mg to about 500 mg, from about 10 mg to about 20 mg, from about 10 mg to about 50 mg, from about 10 mg to about 100 mg, from about 10 mg to about 200 mg, from about 10 mg to about 500 mg, from about 20 mg to about 50 mg, from about 20 mg to about 100 mg, from about 20 mg to about 200 mg, from about 20 mg to about 500 mg, from about 50 mg to about 100 mg, from about 50 mg to about 200 mg, from about 50 mg to about 500 mg, from about 100 mg to about 200 mg, from about 100 mg to about 500 mg, or from about 200 mg to about 500 mg of the modified form of trimetazidine.

The pharmaceutical composition may be formulated such that the maximum level of the modified form of trimetazidine or a metabolite of the modified form of trimetazidine in a sample from the subject is achieved at a defined interval after the pharmaceutical composition has been provided to the subject.

The metabolite of the modified form of trimetazidine may be any compound produced when the modified form of trimetazidine is metabolized in the body. The metabolite of the modified form of trimetazidine may be a compound of Formula (IX), trimetazidine, nicotinic acid, nicotinamide, or nicotinamide riboside.

The interval between the time point at which the composition is provided to the subject and the time point at which the maximum level of the modified form of trimetazidine or metabolite of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

The sample in which the modified form of trimetazidine or metabolite of the modified form of trimetazidine is measured may be any fluid-containing sample from the subject. The sample may be a plasma sample, blood sample, serum sample, saliva sample, urine sample, sputum sample, phlegm sample, stool sample, or gastric sample.

The pharmaceutical composition may be formulated such that, when the composition is provided to a subject, the interval between a first time point at which the maximum level of the modified form of trimetazidine or a metabolite of the modified form of trimetazidine is achieved in a sample from the subject and a second time point at which a half-maximum level of the modified form of trimetazidine or the metabolite of the modified form of trimetazidine is achieved in a sample from the subject is defined.

The interval between the time point at which the maximum level of the modified form of trimetazidine or a metabolite of the modified form of trimetazidine is achieved in a sample from the subject and the time point at which the half-maximal level of the modified form of trimetazidine or the metabolite of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

The pharmaceutical composition may be formulated such that, when the composition is provided to a subject, the maximum level of the modified form of trimetazidine or a metabolite of the modified form of trimetazidine in a sample from the subject does not exceed a defined value.

The maximum level of the modified form of trimetazidine or a metabolite of the modified form of trimetazidine in a sample from the subject may be less than about 6 μg/mL, less than about 5 μg/mL, less than about 4 μg/mL, less than about 3 μg/mL, less than about 2 μg/mL, or less than about 1 μg/mL.

In another aspect, the invention provides pharmaceutical compositions containing a mixture that includes a modified form of trimetazidine and hydroxypropyl methylcellulose (HPMC) in a defined weight ratio.

The mixture may contain the modified form of trimetazidine and HPMC in a weight ratio of about 1:5, about 1:4, about 1:3, about 1:2, about 1:1, about 3:2, about 2:1, about 3:1, about 4:1, about 5:1, from about 1:100 to about 100:1, from about 1:100 to about 50:1, from about 1:100 to about 20:1, from about 1:100 to about 10:1, from about 1:100 to about 5:1, from about 1:100 to about 2:1, from about 1:50 to about 100:1, from about 1:50 to about 50:1, from about 1:50 to about 20:1, from about 1:50 to about 10:1, from about 1:50 to about 5:1, from about 1:50 to about 2:1, from about 1:20 to about 100:1, from about 1:20 to about 50:1, from about 1:20 to about 20:1, from about 1:20 to about 10:1, from about 1:20 to about 5:1, from about 1:20 to about 2:1, from about 1:10 to about 100:1, from about 1:10 to about 50:1, from about 1:10 to about 20:1, from about 1:10 to about 10:1, from about 1:10 to about 5:1, from about 1:10 to about 2:1, from about 1:5 to about 100:1, from about 1:5 to about 50:1, from about 1:5 to about 20:1, from about 1:5 to about 10:1, from about 1:5 to about 5:1, from about 1:5 to about 2:1, from about 1:3 to about 100:1, from about 1:3 to about 50:1, from about 1:3 to about 20:1, from about 1:3 to about 10:1, from about 1:3 to about 5:1, or from about 1:3 to about 2:1.

The modified form of trimetazidine may be any compound that is structurally related to trimetazidine, such as any of those described above.

The mixture may contain multiple polymeric forms of HPMC. The different polymeric forms of HPMC may differ in one or more properties. The different polymeric forms of HPMC may differ in one or more of viscosity, degree of methoxyl substitution, degree of hydroxypropoxyl substitution, and average molecule weight.

Each polymeric form of HPMC may independently have a defined viscosity, degree of methoxyl substitution, degree of hydroxypropoxyl substitution, or average molecule weight, such as any of the values for those parameters described above.

Mixtures containing multiple polymeric forms of HPMC may contain one polymeric form at a defined amount, such as any of the amounts described above.

The mixture may contain a defined amount of the modified form of trimetazidine, such as any of the amounts described above.

The pharmaceutical composition may be formulated for a particular route of administration. The pharmaceutical may be formulated for oral, enteral, intravenous, or rectal administration.

The pharmaceutical composition may be formulated as a unit dosage containing a defined amount of the modified form of trimetazidine, such as any of the amounts described above.

The pharmaceutical composition may be formulated such that a maximum level of the modified form of trimetazidine or a metabolite of the modified form of trimetazidine in a sample from the subject is achieved at a defined interval after the pharmaceutical composition has been provided to the subject.

The metabolite of the modified form of trimetazidine may be any of the compounds described above.

The interval between the time point at which the composition is provided to the subject and the time point at which the maximum level of the modified form of trimetazidine or metabolite of the modified form of trimetazidine is achieved in a sample from the subject may be any of the intervals described above.

The sample in which the modified form of trimetazidine is measured may be any of the samples described above.

The pharmaceutical composition may be formulated such that, when the composition is provided to a subject, the interval between a first time point at which a maximum level of the modified form of trimetazidine or a metabolite of the modified form of trimetazidine is achieved in a sample from the subject and a second time point at which a half-maximum level of the modified form of trimetazidine or the metabolite of the modified form of trimetazidine is achieved in a sample from the subject is defined.

The interval between the time point at which the maximum level of the modified form of trimetazidine or a metabolite of the modified form of trimetazidine is achieved in a sample from the subject and the time point at which the half-maximal level of the modified form of trimetazidine or a metabolite of the modified form of trimetazidine is achieved in a sample from the subject may be any of the intervals described above

The pharmaceutical composition may be formulated such that, when the composition is provided to a subject, the maximum level of the modified form of trimetazidine or a metabolite of the modified form of trimetazidine in a sample from the subject does not exceed a defined value.

The maximum level of the modified form of trimetazidine or a metabolite of the modified form of trimetazidine in a sample from the subject may be any of the values described above.

In another aspect, the invention provides methods of treating a disease, disorder, condition in a subject by providing to the subject a pharmaceutical composition containing a mixture that includes a modified form of trimetazidine and an erodible polymer that promotes swelling of the mixture in an aqueous environment.

The modified form of trimetazidine may be any compound that is structurally related to trimetazidine, such as any of those described above.

The erodible polymer may be any biocompatible polymer that promotes swelling of a mixture containing the modified form of trimetazidine. The polymer may be biodegradable. The polymer may be hydrophilic. The polymer may promote formation of a hydrogel. The polymer may be a cellulose derivative. The polymer may be methylcellulose, hydroxypropyl methylcellulose, ethyl cellulose, hydroxyethyl cellulose, or sodium carboxymethylcellulose.

The mixture may contain multiple polymeric forms of HPMC. The different polymeric forms of HPMC may differ in one or more properties. The different polymeric forms of HPMC may differ in one or more of viscosity, degree of methoxyl substitution, degree of hydroxypropoxyl substitution, and average molecule weight.

Each polymeric form of HPMC may independently have a defined viscosity, degree of methoxyl substitution, degree of hydroxypropoxyl substitution, or average molecule weight, such as any of the values for those parameters described above.

Mixtures containing multiple polymeric forms of HPMC may contain one polymeric form at a defined amount, such as any of the amounts described above.

The mixture may contain a defined amount of the modified form of trimetazidine, such as any of the amounts described above.

The pharmaceutical composition may be provided to the subject by a particular route of administration. The pharmaceutical may be provided to the subject orally, enterally, intravenously, or rectally.

The method may yield a maximum level of the modified form of trimetazidine or a metabolite of the modified form of trimetazidine in a sample from the subject at a defined interval after the pharmaceutical composition has been provided to the subject.

The metabolite of the modified form of trimetazidine may be any compound produced when the modified form of trimetazidine is metabolized in the body. The metabolite of the modified form of trimetazidine may be a compound of Formula (IX), trimetazidine, nicotinic acid, nicotinamide, or nicotinamide riboside.

The interval between the time point at which the composition is provided to the subject and the time point at which the maximum level of the modified form of trimetazidine or metabolite of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

The sample in which the modified form of trimetazidine or metabolite of the modified form of trimetazidine is measured may be any fluid-containing sample from the subject. The sample may be a plasma sample, blood sample, serum sample, saliva sample, urine sample, sputum sample, phlegm sample, stool sample, or gastric sample.

The method may yield a defined interval between a first time point at which a maximum level of the modified form of trimetazidine or a metabolite of the modified form of trimetazidine is achieved in a sample from the subject and a second time point at which a half-maximum level of the modified form of trimetazidine or the metabolite of the modified form of trimetazidine is achieved in a sample from the subject.

The interval between the time point at which the maximum level of the modified form of trimetazidine or a metabolite of the modified form of trimetazidine is achieved in a sample from the subject and the time point at which the half-maximal level of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

The method may yield a maximum level of the modified form of trimetazidine or a metabolite of the modified form of trimetazidine in a sample from the subject that does not exceed a defined value.

The maximum level of the modified form of trimetazidine or a metabolite of the modified form of trimetazidine in a sample from the subject may be less than about 6 μg/mL, less than about 5 μg/mL, less than about 4 μg/mL, less than about 3 μg/mL, less than about 2 μg/mL, or less than about 1 μg/mL.

The disease, disorder, or condition may be any condition that can be ameliorated by improving cardiac mitochondrial function. The disease, disorder, or condition may be a cardiovascular condition. The disease, disorder, or condition may be aneurysm, angina, atherosclerosis, cardiomyopathy, cerebral vascular disease, congenital heart disease. coronary artery disease (CAD), coronary heart disease, diabetic cardiomyopathy, heart attack, heart disease, heart failure, high blood pressure (hypertension), ischemic heart disease, pericardial disease, peripheral arterial disease, refractory angina, rheumatic heart disease, stable angina, stroke, transient ischemic attack, unstable angina, or valvular heart disease.

In another aspect, the invention provides methods of treating a disease, disorder, condition in a subject by providing to the subject a pharmaceutical composition containing a mixture that includes a modified form of trimetazidine and hydroxypropyl methylcellulose (HPMC), wherein the mixture comprises the modified form of trimetazidine and HPMC in a defined weight ratio.

The mixture may contain the modified form of trimetazidine and HPMC in one of the ratios described above.

The modified form of trimetazidine may be any compound that is structurally related to trimetazidine, such as any of those described above.

The mixture may contain multiple polymeric forms of HPMC. The different polymeric forms of HPMC may differ in one or more properties. The different polymeric forms of HPMC may differ in one or more of viscosity, degree of methoxyl substitution, degree of hydroxypropoxyl substitution, and average molecule weight.

Each polymeric form of HPMC may independently have a defined viscosity, degree of methoxyl substitution, degree of hydroxypropoxyl substitution, or average molecule weight, such as any of the values for those parameters described above.

Mixtures containing multiple polymeric forms of HPMC may contain one polymeric form at a defined amount, such as any of the amounts described above.

The mixture may contain a defined amount of the modified form of trimetazidine, such as any of the amounts described above.

The pharmaceutical composition may be provided to the subject by a particular route of administration. The pharmaceutical may be provided to the subject orally, enterally, intravenously, or rectally.

The method may yield a maximum level of the modified form of trimetazidine or a metabolite of the modified form of trimetazidine in a sample from the subject at a defined interval after the pharmaceutical composition has been provided to the subject.

The metabolite of the modified form of trimetazidine may be any of the compounds described above.

The interval between the time point at which the composition is provided to the subject and the time point at which the maximum level of the modified form of trimetazidine or metabolite of the modified form of trimetazidine is achieved in a sample from the subject may be any of the intervals described above.

The sample in which the modified form of trimetazidine is measured may be any of the samples described above.

The method may yield a defined interval between a first time point at which a maximum level of the modified form of trimetazidine or a metabolite of the modified form of trimetazidine is achieved in a sample from the subject and a second time point at which a half-maximum level of the modified form of trimetazidine or the metabolite of the modified form of trimetazidine is achieved in a sample from the subject.

The interval between the time point at which the maximum level of the modified form of trimetazidine or a metabolite of the modified form of trimetazidine is achieved in a sample from the subject and the time point at which the half-maximal level of the modified form of trimetazidine or a metabolite of the modified form of trimetazidine is achieved in a sample from the subject may be any of the intervals described above.

The method may yield a maximum level of the modified form of trimetazidine or a metabolite of the modified form of trimetazidine in a sample from the subject that does not exceed a defined value.

The maximum level of the modified form of trimetazidine or a metabolite of the modified form of trimetazidine in a sample from the subject may be any of the values described above.

The disease, disorder, or condition may be any condition that can be ameliorated by improving cardiac mitochondrial function, such as any of those described above.

### BRIEF DESCRIPTION OF THE DRAWINGS

FIG. 1 is a schematic of the pathways of hydrolysis and metabolism of CV-8972.

FIG. 2 is graph of the aqueous solubility of CV-8972 as a function of pH generated via dynamic methods due to instability.

FIG. 3 is a process flow diagram for manufacture of the CV-8972 prototype dry compression MR tablet formulations.

FIG. 4 is a process flow diagram for manufacture of the CV-8972 200 and 50 mg wet granulated, MR Tablet formulations used for pilot lots.

FIG. 5 is graph showing the dissolution profile of 50 mg and 200 mg 4-hour modified release hydrogel tablets in 0.1 M HCl pH 1.0 at 37° C.

FIG. 6 is graph showing the dissolution profile of 50 mg and 200 mg 8-hour modified release hydrogel tablets in 0.1 M HCl pH 1.0 at 37° C.

FIG. 7 is graph showing the dissolution profiles of 200 mg 8-hour modified release hydrogel tablets in 0.1 M HCl pH 1.0 at 37° C. following storage under indicated conditions.

FIG. 8 is a graph showing CV-8814 plasma levels in dogs following oral dosing of 200 mg (free base equivalent) of CV-8972 under fasted conditions.

FIG. 9 is a graph showing the simulated combined plasma levels of CV-8814 and trimetazidine projected in humans following oral dosing 200 mg IMB-1018972 MR tablets as 8-hour release tablets under MAD dosing (QD and BID) and fed conditions.

FIG. 10 is a table of the disposition of subjects of an FIH study of IMB-1018972.

FIG. 11 is a table of assessments given for the Single-Dose MR Part of an FIH study of IMB-1018972

FIG. 12 is a table of assessments given for the Multiple-Dose MR part of an FIE study of IMB-1018972.

FIG. 13 is a table of analysis data sets for the Single-Dose MR Part of an FIH study of IMB-1018972.

FIG. 14 a table of analysis data sets for the Multiple-Dose MR Part of an FIE study of IMB-1018972.

FIG. 15 is a table of a summary of demographic characteristics—Single-Dose MR Part (Safety Set) of an FIH study of IMB-1018972.

FIG. 16 is a table of a summary of demographic characteristics—Multiple-Dose MR Part (Safety Set) of an FIH study of IMB-1018972.

FIG. 17 is a table of the Extent of Exposure—Single-Dose MR Part (Safety Set) of an FIE study of IMB-1018972.

FIG. 18 is a table of the Extent of Exposure—Multiple-Dose MR Part (Safety Set) of an FIE study of IMB-1018972.

FIG. 19 is a graph of Geometric Mean IMB-1028814 Plasma Concentration-Time Profiles (Linear)—Single-Dose MR Part (PK Set) of an FIH study of IMB-1018972.

FIG. 20 is a graph of Geometric Mean IMB-1028814 Plasma Concentration-Time Profiles (Semi-Logarithmic Scale)—Single-Dose MR Part (PK Set) of an FIH study of IMB-1018972.

FIG. 21 is a graph of Geometric Mean Trimetazidine Plasma Concentration-Time Profiles (Linear)—Single-Dose MR Part (PK Set) of an FIH study of IMB-1018972.

FIG. 22 is a graph of Geometric Mean Trimetazidine Plasma Concentration-Time Profiles (Semi-Logarithmic Scale)—Single-Dose MR Part (PK Set) of an FIH study of IMB-1018972.

FIG. 23 is a graph of Geometric Mean IMB-1028814+Trimetazidine Plasma Concentration-Time Profiles (Linear)—Single-Dose MR Part (PK Set) of an FIH study of IMB-1018972.

FIG. 24 is a graph of Geometric Mean IMB-1028814+Trimetazidine Plasma Concentration-Time Profiles (Semi-Logarithmic Scale)—Single-Dose MR Part (PK Set) of an FIH study of IMB-1018972.

FIG. 25 is a table of Summary Statistics Geometric Mean [Range]) of IMB-1028814, Trimetazidine, and IMB-1028814+Trimetazidine Plasma Pharmacokinetic Parameters—Single-Dose MR Part (PK Set) of an FIH study of IMB-1018972.

FIG. 26 is a table of Exploratory Analysis of Food Effect for IMB-1028814 and Trimetazidine following Administration of 200 mg 8-hour MR IMB-1018972—Single-Dose MR Part (PK Set) of an FIH study of IMB-1018972.

FIG. 27 is a graph of Geometric Mean IMB-1028814 Plasma Concentration-Time Profiles from Day 1 through Day 5 (Linear)—Multiple-Dose MR Part (PK Set) of an FIH study of IMB-1018972.

FIG. 28 is a graph of Geometric Mean IMB-1028814 Plasma Concentration-Time Profiles from Day 1 through Day 5 (Semi-Logarithmic Scale)—Multiple-Dose MR Part (PK Set) of an FIH study of IMB-1018972.

FIG. 29 is a graph of Geometric Mean Trimetazidine Plasma Concentration-Time Profiles from Day 1 through Day 5 (Linear)—Multiple-Dose MR Part (PK Set) of an FIH study of IMB-1018972.

FIG. 30 is a graph of Geometric Mean Trimetazidine Plasma Concentration-Time Profiles from Day 1 through Day 5 (Semi-Logarithmic Scale)—Multiple-Dose MR Part (PK Set) of an FIH study of IMB-1018972.

FIG. 31 is a graph of Geometric Mean IMB-1028814+Trimetazidine Plasma Concentration-Time Profiles from Day 1 through Day 5 (Linear)—Multiple-Dose MR Part (PK Set) of an FIH study of IMB-1018972.

FIG. 32 is a graph of Geometric Mean IMB-1028814+Trimetazidine Plasma Concentration-Time Profiles from Day 1 through Day 5 (Semi-Logarithmic Scale)—Multiple-Dose MR Part (PK Set) of an FIH study of IMB-1018972.

FIG. 33 is a graph of Geometric Mean IMB-1028814 Plasma Concentration-Time Profiles after Day 1 through Day 5 (Linear)—Multiple-Dose MR Part (PK Set) of an FIH study of IMB-1018972.

FIG. 34 is a graph of Geometric Mean IMB-1028814 Plasma Concentration-Time Profiles after Day 1 through Day 5 (Semi-Logarithmic Scale)—Multiple-Dose MR Part (PK Set) of an FIE study of IMB-1018972.

FIG. 35 is a graph of Geometric Mean Trimetazidine Plasma Concentration-Time Profiles after Day 1 through Day 5 (Linear)—Multiple-Dose MR Part (PK Set) of an FIH study of IMB-1018972.

FIG. 36 is a graph of Geometric Mean Trimetazidine Plasma Concentration-Time Profiles after Day 1 through Day 5 (Semi-Logarithmic Scale)—Multiple-Dose MR Part (PK Set) of an FIE study of IMB-1018972.

FIG. 37 is a graph of Geometric Mean IMB-1028814+Trimetazidine Plasma Concentration-Time Profiles after Day 1 through Day 5 (Linear)—Multiple-Dose MR Part (PK Set) of an FIH study of IMB-1018972.

FIG. 38 is a graph of Geometric Mean IMB-1028814+Trimetazidine Plasma Concentration-Time Profiles after Day 1 through Day 5 (Semi-Logarithmic Scale)—Multiple-Dose MR Part (PK Set) of an FIH study of IMB-1018972.

FIG. 39 is a table of Summary Statistics Geometric Mean [Range]) of IMB-1028814, Trimetazidine, and IMB-1028814+Trimetazidine Plasma Pharmacokinetic Parameters—Multiple-Dose MR Part (PK Set) of an FIH study of IMB-1018972.

FIG. 40A and FIG. 40B is a table Summary of All TEAEs by System Organ Class, Preferred Term and Treatment—Single-Dose MR Part (Safety Set) of an FIE study of IMB-1018972.

FIG. 41 is a table Summary of All TEAEs by System Organ Class, Preferred Term and Treatment—Single-Dose MR Part (Safety Set) of an FIH study of IMB-1018972.

FIG. 42 is a table Summary of All TEAEs by Treatment, Relationship, and Severity-Single-Dose MR Part (Safety Set) of an FIH study of IMB-1018972.

FIG. 43 is a table Summary of All TEAEs by Treatment, Relationship, and Severity—Multiple-Dose MR Part (Safety Set) of an FIH study of IMB-1018972.

### DETAILED DESCRIPTION

The invention provides pharmaceutical compositions that contain a mixture of a modified form of trimetazidine, such as CV-8972, and an erodible polymer, such as hydroxypropyl methylcellulose (HPMC). The polymer absorbs water to promote swelling of the mixture when the compositions are in an aqueous environment, such as the gastrointestinal (GI) tract. In addition, the polymer gradually breaks down in an aqueous milieu to allow controlled release of the modified form of trimetazidine, which then breaks-down into niacin and CV-8814 in a controlled manner, thereby providing a controlled and modified release of niacin into the body. Consequently, when the compositions are administered orally to a subject, levels of active pharmaceutical ingredient (API) in the blood exhibit both lower peaks and longer durations above the minimum therapeutic threshold than when the same dosage of the same modified form of trimetazidine is provided in a conventional formulation and side effects from niacin are minimized.

The modified-release formulations of the invention confer several advantages over prior formulations for oral delivery of modified forms of trimetazidine. First, due to their extended maintenance of the API above a threshold value in the blood, the pharmaceutical compositions of the invention can be administered less frequently than prior compositions. For example, oral formulations containing CV-8972 provided herein are suitable for once-per-day or twice-per-day dosing regimens. In addition, because peak levels of API in the blood are as much as 50% lower than levels achieved with prior compositions, the formulations of the invention reduce side effects that result from niacin and interaction between the API and unintended targets in vivo. Finally, the aforementioned attributes improve the overall therapeutic efficacy of modified forms of trimetazidine, such as CV-8972.

### Modified Forms of Trimetazidine

The invention provides compositions that contain mixtures that include modified forms of trimetazidine. Trimetazidine has the following structure:

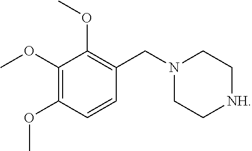 [SMILES: COc1ccc(CN2CCNCC2)c(OC)c1OC]

Trimetazidine is described as the first cytoprotective anti-ischemic agent developed and has long been used to treat angina.

Trimetazidine promotes glucose oxidation by inhibiting oxidation of fatty acids. Glucose oxidation and fatty acid oxidation are energy-producing metabolic pathways that compete with each other for substrates. In glucose oxidation, glucose is broken down to pyruvate via glycolysis in the cytosol of the cell. Pyruvate then enters the mitochondria, where it is converted to acetyl coenzyme A (acetyl-CoA). In beta-oxidation of fatty acids, which occurs in the mitochondria, two-carbon units from long-chain fatty acids are sequentially converted to acetyl-CoA. The remaining steps in energy production from oxidation of glucose or fatty acids are common to the two pathways. Briefly, they include breakdown of acetyl-CoA to carbon dioxide via the citric acid cycle, the concomitant generation of a proton gradient across the mitochondrial inner membrane via a series of oxygen-dependent electron transport reactions, and the use of the energy potential in the proton gradient to drive ATP synthesis. Trimetazidine inhibits oxidation of fatty acids by blocking long-chain 3-ketoacyl-CoA thiolase, thus causing cells to rely on glucose oxidation to support energy production.

Forcing cardiac mitochondria to rely on oxidation of glucose rather fatty acids as an energy source provides a therapeutic benefit for many patients with cardiovascular conditions. In certain types of heart disease, the overall efficiency of energy production by cardiac mitochondria is diminished due in part to an increased reliance on fatty acid oxidation over glucose oxidation. Glucose oxidation is a more efficient pathway for energy production, as measured by the number of ATP molecules produced per 02 molecule consumed, than is fatty acid oxidation. Thus, overall cardiac efficiency can be increased by agents that promote glucose oxidation, such as trimetazidine.

CV-8972 was recently identified as a trimetazidine-derivative having improved pharmacological properties. CV-8972 has the IUPAC name 2-[4-[(2,3,4-trimethoxyphenyl)methyl]piperazin-1-yl]ethyl pyridine-3-carboxylate and the structure of Formula (X):

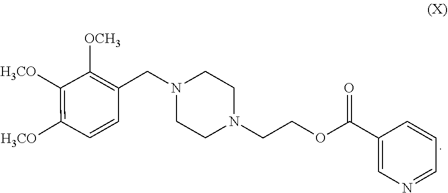 [SMILES: COc1ccc(CN2CCN(CCOC(=O)c3cccnc3)CC2)c(OC)c1OC]

When CV-8972 is administered to a subject, it is initially broken into nicotinic acid and CV-8814, which has the IUPAC name 2-[4-[(2,3,4-trimethoxyphenyl)methyl]piperazin-1-yl]ethanol and the structure of Formula (IX):

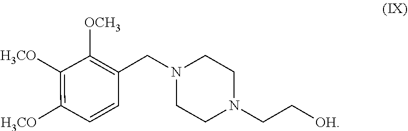 [SMILES: COc1ccc(CN2CCN(CCO)CC2)c(OC)c1OC]

CV-8814 is a hydroxyethyl derivative of trimetazidine, and the hydroxyethyl group is subsequently removed in the body to provide trimetazidine. CV-8972 and its metabolic products are described in U.S. Pat. No. 10,556,013, the contents of which are incorporated herein by reference.

The improved therapeutic properties of CV-8972 are due in part to the effect of nicotinic acid. Nicotinic acid serves as a precursor for synthesis of nicotinamide adenine dinucleotide (NAD⁺), the oxidized form of an essential coenzyme in the mitochondrial electron transport reaction. Supplying a NAD⁺ precursor ensures that mitochondrial redox reactions occur robustly to drive ATP synthesis, regardless of whether oxidation of glucose or fatty acids is used to feed the citric acid cycle. Thus, the nicotinic acid product of CV-8972 promotes mitochondrial respiration.

The stepwise breakdown of CV-8972 to CV-8814 and then to trimetazidine also contributes to the improved therapeutic properties of CV-8972. Like trimetazidine, CV-8814 inhibits 3-ketoacyl-CoA thiolase, so CV-8972 delivers two different active pharmaceutical ingredients (APIs). However, CV-8814 does not produce the same undesirable side effects as trimetazidine. In addition, due to the sequential metabolism of CV-8972, the level of circulating trimetazidine following a dose of CV-8972 is much lower than the level following of comparable dose of trimetazidine itself. Therefore, compared to unadulterated trimetazidine, CV-8972 provides a more sustained level of circulating API and fewer side effects.

Other modified forms of trimetazidine that may be used in compositions of the invention are described in, for example, U.S. Pat. Nos. 4,100,285 and 4,574,156, the contents of each of which are incorporated herein by reference.

### Erodible Polymers, Including HPMC

The invention provides compositions that contain mixtures that include erodible polymers that promote swelling of the mixture in an aqueous environment. An erodible polymer is any polymer that breaks down inside the body within a physiologically relevant time frame. The erodible polymer may have other characteristics that promote the gradual release of the modified form of trimetazidine from the mixture. For example and without limitation, the polymer may be one or more of the following: biocompatible, i.e., not harmful to living tissue; hydrophilic; hygroscopic; tending to form a hydrogel.

Without wishing to be bound by theory, the polymer-containing mixtures may promote gradual release by one or more mechanisms. For example, swelling of the mixture by absorption of water may facilitate diffusion of the modified form of trimetazidine from the mixture. Degradation of the polymer may also allow the modified form of trimetazidine to be released from the mixture. Osmotic pressure due the high concentration gradient of compound between the inside and outside of the mixture may also contribute to diffusion of the modified form of trimetazidine from the mixture.

For example and without limitation, the polymer may be a cellulose derivative, a gelatin derivative, e.g., a cross-linked gelatin derivative, or a polyester derivative.

Derivatives of cellulose, is a linear chain β(1→4) linked D-glucose units, include polymers that contain substitutions on one of more of the hydroxyl groups of each glucose unit. Substituents may be organic or inorganic and are typically attached via ester or ether linkages. Cellulose ester derivatives include carboxymethyl cellulose (CMC), e.g., sodium carboxymethyl cellulose, ethyl cellulose, ethyl hydroxyethyl cellulose, ethyl methyl cellulose, hydroxyethyl cellulose, hydroxyethyl methyl cellulose, hydroxypropyl cellulose (HPC), hydroxypropyl methylcellulose (HPMC), and methylcellulose. Cellulose ether derivatives include cellulose acetate, cellulose acetate butyrate, cellulose acetate propionate, cellulose propionate, cellulose sulfate, cellulose triacetate, and nitrocellulose. The use of cellulose-based polymers to form biodegradable hydrogels is known in the art and described in, for example, Sannino, et al., Biodegradable Cellulose-based Hydrogels: Design and applications, Materials 2009, 2, 353-373; doi:10.3390/ma2020353, the contents of which are incorporated herein by reference.

The mixture may contain multiple polymers or multiple polymeric forms of the same polymer. For example, HPMC polymeric forms may differ in a variety of physical properties, including viscosity, degree of methoxyl substitution, degree of hydroxypropoxyl substitution, or average molecule weight.

The viscosity of a HMPC polymeric form may be determined by testing under standard conditions, including the concentration of HMPC in the solution and the temperature of the solution. For example and without limitation, the HPMC concentration may be 1%, 1.5%, 2%, 2.5%, or 3%. For example and without limitation, the temperature of the solution may be 15° C., 16° C., 17° C., 18° C., 19° C., 20° C., 21° C., 22° C., 23° C., 24° C., or 25° C.

A polymeric form of a cellulose derivative, such as HPMC, may have a defined viscosity. For example and without limitation, a polymeric form of HPMC may have a viscosity of from about 2 cP to about 4 cP, from about 4 cP to about 6 cP, from about 5 cP to about 8 cP, from about 12 cP to about 18 cP, from about 40 cP to about 60 cP, from about 80 cP to about 120 cP, from about 300 cP to about 500 cP, from about 1200 cP to about 2400 cP, from about 2500 cP to about 5000 cP, from about 9000 cP to about 18,000 cP, from about 12,000 cP to about 24,000 cP, from about 12,000 cP to about 24,000 cP, from about 75,000 cP to about 150,000 cP, at least about 2 cP at least about 4 cP at least about 5 cP at least about 12 cP at least about 40 cP at least about 80 cP at least about 300 cP at least about 1200 cP at least about 2500 cP at least about 9000 cP at least about 12,000 cP at least about 12,000 cP at least about 75,000 cP less than about 4 cP, less than about 6 cP, less than about 8 cP, less than about 18 cP, less than about 60 cP, less than about 120 cP, less than about 500 cP, less than about 2400 cP, less than about 5000 cP, less than about 18,000 cP, less than about 24,000 cP, less than about 24,000 cP, or less than about 150,000 cP for a 2% aqueous solution of the polymeric form at 20° C.

Polymeric forms of cellulose derivatives, such as HPMC, may vary in their degree of substitution of the glucose units. The degree of substitution may be expressed as a weight percentage of the substituent or as a molar ratio of substituent to glucose unit. For a cellulose derivative that has two different substituents, such as HPMC, the polymeric form may be described by the degree of substitution for each substituent.

Each polymeric form of HPMC may independently have a defined degree of methoxyl substitution. For example and without limitation, the degree of methoxyl substitution may be from about 19% to about 24%, from about 22% to about 24%, from about 27% to about 30%, from about 27% to about 30%, or from about 28% to about 32%.

Each polymeric form of HPMC may independently have a defined degree of hydroxypropoxyl substitution. For example and without limitation, the degree of hydroxypropoxyl substitution may be from about 4% to about 8%, from about 7% to about 10%, from about 7% to about 12%, from about 8% to about 10%, from about 8% to about 11%, or from about 9% to about 12%.

Each polymeric form of HPMC may independently have a defined average molecular weight. The average molecular weight may be about 10 kDa, about 13 kDa, about 20 kDa, about 26 kDa, about 41 kDa, about 63 kDa, about 86 kDa, about 110 kDa, about 120 kDa, about 140 kDa, about 180 kDa, or about 220 kDa.

When multiple forms of a polymer, such as HPMC, are present, one or more polymeric forms may be present in a defined amount. For example and without limitation, a polymer, such as HPMC, may contain about 50%, about 60%, about 70%, about 80%, about 90%, about 95%, about 96%, about 97%, about 98%, about 99%, at least 50%, at least 60%, at least 70%, at least 80%, at least 90%, at least 95%, at least 96%, at least 97%, at least 98%, or at least 99% by weight of one polymeric form.

### Pharmaceutical Compositions

Pharmaceutical compositions of the invention include modified-release formulations that contain one or more modified forms of trimetazidine. The formulations contain mixtures that include one or more modified forms of trimetazidine and one or more erodible polymers that promote swelling of the mixture in an aqueous environment. The hygroscopic and erodible properties of the polymers may allow the mixture to form a hydrogel that slowly breaks down in the digestive tract of the subject. Consequently, the mixture promotes the steady release of the modified form of trimetazidine and metabolic products thereof into circulation.

The mixture may contain a defined amount of the modified form of trimetazidine. The mixture may contain at least 5%, at least 10%, at least 20%, at least 30%, at least 40%, at least 50%, at least 60%, at least 70%, or at least 80% by weight of the modified form of trimetazidine.

The mixture may contain the modified form of trimetazidine and the polymer in a defined weight ratio. For example and without limitation, the mixture may contain the modified form of trimetazidine and the polymer in a weight ratio of about 1:5, about 1:4, about 1:3, about 1:2, about 1:1, about 3:2, about 2:1, about 3:1, about 4:1, about 5:1, from about 1:100 to about 100:1, from about 1:100 to about 50:1, from about 1:100 to about 20:1, from about 1:100 to about 10:1, from about 1:100 to about 5:1, from about 1:100 to about 2:1, from about 1:50 to about 100:1, from about 1:50 to about 50:1, from about 1:50 to about 20:1, from about 1:50 to about 10:1, from about 1:50 to about 5:1, from about 1:50 to about 2:1, from about 1:20 to about 100:1, from about 1:20 to about 50:1, from about 1:20 to about 20:1, from about 1:20 to about 10:1, from about 1:20 to about 5:1, from about 1:20 to about 2:1, from about 1:10 to about 100:1, from about 1:10 to about 50:1, from about 1:10 to about 20:1, from about 1:10 to about 10:1, from about 1:10 to about 5:1, from about 1:10 to about 2:1, from about 1:5 to about 100:1, from about 1:5 to about 50:1, from about 1:5 to about 20:1, from about 1:5 to about 10:1, from about 1:5 to about 5:1, from about 1:5 to about 2:1, from about 1:3 to about 100:1, from about 1:3 to about 50:1, from about 1:3 to about 20:1, from about 1:3 to about 10:1, from about 1:3 to about 5:1, or from about 1:3 to about 2:1.

The pharmaceutical composition may be formulated for a particular route of administration. The pharmaceutical may be formulated for oral, enteral, intravenous, or rectal administration.

The pharmaceutical composition may be formulated as a unit dosage containing a defined amount of the modified form of trimetazidine. The unit dosage may contain about 5 mg, about 10 mg, about 20 mg, about 50 mg, about 100 mg, about 200 mg, about 500 mg, from about 5 mg to about 10 mg, from about 5 mg to about 20 mg, from about 5 mg to about 50 mg, from about 5 mg to about 100 mg, from about 5 mg to about 200 mg, from about 5 mg to about 500 mg, from about 10 mg to about 20 mg, from about 10 mg to about 50 mg, from about 10 mg to about 100 mg, from about 10 mg to about 200 mg, from about 10 mg to about 500 mg, from about 20 mg to about 50 mg, from about 20 mg to about 100 mg, from about 20 mg to about 200 mg, from about 20 mg to about 500 mg, from about 50 mg to about 100 mg, from about 50 mg to about 200 mg, from about 50 mg to about 500 mg, from about 100 mg to about 200 mg, from about 100 mg to about 500 mg, or from about 200 mg to about 500 mg of the modified form of trimetazidine.

The pharmaceutical composition may be formulated such that it produces a defined value for one or more parameters, as described below in relation to methods of the invention. For example and without limitation, the parameter may be Cₘₐₓ, the interval between administration and achieving Cₘₐₓ, T_{1/2}, or AUC.

Pharmaceutical compositions of the invention may contain excipients. For example and without limitation, the composition may contain sweetening agents, flavoring agents, coloring agents, or preserving agents. The compositions may contain one or more of mannitol, starch, and magnesium stearate.

### Methods of Treatment

The invention provides methods of treating a disease, disorder, condition in a subject by providing any of the compositions described above. The modified release formulations of the invention provide steadier release of the modified form of trimetazidine than do conventional formulations. The superior release profile may be reflected in one or more parameters described below.

One parameter that may be used to distinguish formulations of the invention from other compositions that contain the same dosage of modified form of trimetazidine is Cₘₐₓ, the maximum level of a drug or metabolite of the drug in a sample following the administration of a dose of the drug but prior to administration of a second dose. The formulations of the invention may yield Cₘₐₓ values that are lower than those produced by conventional formulations that contain the same dosage. In methods of the invention, the lower Cₘₐₓ may be expressed in relative terms, e.g., by comparison to a Cₘₐₓ resulting from administration of another formulation, or in absolute terms, e.g., by comparison to a defined threshold value. The Cₘₐₓ may be for the modified form of trimetazidine or a metabolite of the compound. For example, following administration of a composition containing the compound of Formula (X), the Cₘₐₓ of the compound of Formula (X) may be determined, or the Cₘₐₓ of the compound of Formula (IX), trimetazidine, or nicotinic acid may be determined.

For example and without limitation, the maximum level of the modified form of trimetazidine or a metabolite of the modified form of trimetazidine in a sample from the subject may be less than about 20 μg/mL, less than about 15 μg/mL, less than about 12 μg/mL, less than about 10 μg/mL, less than about 8 μg/mL, less than about 6 μg/mL, less than about 5 μg/mL, less than about 4 μg/mL, less than about 3 μg/mL, less than about 2 μg/mL, less than about 1 μg/mL, less than about 0.8 μg/mL, less than about 0.6 μg/mL, less than about 0.4 μg/mL, less than about 0.2 μg/mL, or less than about 0.1 μg/mL.

For example and without limitation, the maximum level of the modified form of trimetazidine or a metabolite of the modified form of trimetazidine in a sample from the subject may be less than about 10%, less than about 20%, less than about 30%, less than about 40%, less than about 50%, less than about 60%, less than about 70%, less than about 80%, less than about 90% of the maximum level resulting from administration of a different composition that contains the same amount of the modified form of trimetazidine.

Another parameter that may be used to distinguish formulations of the invention from other compositions that contain the same dosage of modified form of trimetazidine is the interval between administration of the composition to the subject and the time point at which the modified form of trimetazidine or a metabolite of the modified form of trimetazidine achieves its Cₘₐₓ in a sample from the subject. The modified-release formulations of the invention may yield longer intervals to Cₘₐₓ than do other formulations. The interval to Cₘₐₓ may be expressed in relative terms, e.g., by comparison to the interval for another formulation, or in absolute terms, e.g., by comparison to a defined period of time.

For example and without limitation, the interval between the time point at which the composition is provided to the subject and the time point at which the maximum level of the modified form of trimetazidine or metabolite of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

For example and without limitation, the interval between the time point at which the composition is provided to the subject and the time point at which the maximum level of the modified form of trimetazidine or metabolite of the modified form of trimetazidine is achieved in a sample from the subject may be at least 10%, least 20%, least 30%, least 40%, least 50%, least 60%, least 70%, least 80%, least 90%, least 100%, least 120%, least 150%, least 200%, least 250%, least 300%, least 400% greater than the interval following administration of another composition containing the same dosage of modified form of trimetazidine.

Another parameter that may be used to distinguish formulations of the invention from other compositions that contain the same dosage of modified form of trimetazidine is T_{1/2}, the interval between the time point at Cₘₐₓ of the modified form of trimetazidine or a metabolite of the modified form of trimetazidine is achieved and the time point at which the concentration of the compound or metabolite reaches its half-maximum value. The modified-release formulations of the invention may yield higher T_{1/2} values, i.e., longer intervals, than those produced by other formulations containing the same dosage of modified form of trimetazidine. The T_{1/2} may be expressed in relative terms, e.g., by comparison to the T_{1/2} for another formulation, or in absolute terms, e.g., by comparison to a defined period of time.

For example and without limitation, the interval between the time point at which the maximum level of the modified form of trimetazidine or a metabolite of the modified form of trimetazidine is achieved in a sample from the subject and the time point at which the half-maximal level of the modified form of trimetazidine is achieved in a sample from the subject may be at least 1 hour, at least 1.5 hours, at least 2 hours, at least 2.5 hours, at least 3 hours, at least 4 hours, at least 5 hours, at least 6 hours, at least 7 hours, at least 8 hours, from about 1 hour to about 8 hours, from about 2 hours to about 8 hours, from about 3 hours to about 8 hours, from about 4 hours to about 8 hours, from about 1 hour to about 7 hours, from about 2 hours to about 7 hours, from about 3 hours to about 7 hours, from about 4 hours to about 7 hours, from about 1 hour to about 6 hours, from about 2 hours to about 6 hours, from about 3 hours to about 6 hours, from about 4 hours to about 6 hours, from about 1 hour to about 5 hours, from about 2 hours to about 5 hours, from about 3 hours to about 5 hours, or from about 4 hours to about 5 hours.

For example and without limitation, the interval between the time point at which the maximum level of the modified form of trimetazidine or a metabolite of the modified form of trimetazidine is achieved in a sample from the subject and the time point at which the half-maximal level of the modified form of trimetazidine is achieved in a sample from the subject may be at least 10%, least 20%, least 30%, least 40%, least 50%, least 60%, least 70%, least 80%, least 90%, least 100%, least 120%, least 150%, least 200%, least 250%, least 300%, least 400% greater than the interval following administration of another composition containing the same dosage of modified form of trimetazidine.

Another parameter that may be used to distinguish formulations of the invention from other compositions that contain the same dosage of modified form of trimetazidine is area under the curve (AUC), the definite integral of concentration of the compound or metabolite as a function of time. The modified-release formulations of the invention may yield higher AUC values than those produced by other formulations containing the same dosage of modified form of trimetazidine. The AUC may be expressed in relative terms, e.g., by comparison to the AUC for another formulation, or in absolute terms, e.g., by comparison to a defined threshold.

For example and without limitation, the AUC the modified form of trimetazidine or a metabolite of the modified form of trimetazidine may be at least 10%, least 20%, least 30%, least 40%, least 50%, least 60%, least 70%, least 80%, least 90%, least 100%, least 120%, least 150%, least 200%, least 250%, least 300%, least 400% greater than the AUC following administration of another composition containing the same dosage of modified form of trimetazidine.

The sample in which the modified form of trimetazidine is measured may be any fluid-containing sample from the subject. The sample may be a plasma sample, blood sample, serum sample, saliva sample, urine sample, sputum sample, phlegm sample, stool sample, or gastric sample.

The composition may be provided to the subject by a particular route of administration. The pharmaceutical may be provided to the subject orally, enterally, intravenously, or rectally.

The composition may be provided according to a dosing regimen. A dosing regimen may include one or more of a dosage, dosing frequency, and duration.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least 1 week, at least 2 weeks, at least 3 weeks, at least 4 weeks, at least 6 weeks, at least 8 weeks, at least 10 weeks, at least 12 weeks, at least 4 months, at least 5 months, at least 6 months, at least 8 months, at least 10 months, at least 12 months or more.

### Diseases, Disorders, and Conditions

The methods of the invention may be used to treat a disease, disorder, or condition in a subject. The disease, disorder, or condition may be any condition that can be ameliorated by improving cardiac mitochondrial function. The disease, disorder, or condition may be a cardiovascular condition. The disease, disorder, or condition may be aneurysm, angina, atherosclerosis, cardiomyopathy, cerebral vascular disease, congenital heart disease. coronary artery disease (CAD), coronary heart disease, diabetic cardiomyopathy, heart attack, heart disease, heart failure, high blood pressure (hypertension), ischemic heart disease, pericardial disease, peripheral arterial disease, refractory angina, rheumatic heart disease, stable angina, stroke, transient ischemic attack, unstable angina, or valvular heart disease.

Angina pectoris (angina) is chest pain or pressure that is typically due to insufficient blood flow to the heart muscle. The pain or discomfort is retrosternal or left-sided and may radiate to the left arm, neck, jaw, or back. Several classifications of angina are known.

Stable angina, also called effort angina, is related to myocardial ischemia. In stable angina, chest discomfort and associated symptoms are usually triggered by some physical activity, such as running or walking, but symptoms are minimal or non-existent when the patient is at rest or has taken sublingual nitroglycerin. Symptoms typically abate several minutes after activity and recur when activity resumes. Symptoms may also be induced by cold weather, heavy meals, and emotional stress.

Unstable angina is angina that changes or worsens. Unstable angina has at least one of the following features: (1) it occurs at rest or with minimal exertion, usually lasting more than 10 minutes, (2) it is severe and of new onset, i.e., within the prior 4-6 weeks, and (3) it occurs with a crescendo pattern, i.e., distinctly more severe, prolonged, or frequent than before.

Cardiac syndrome X, also called microvascular angina, is angina-like chest pain, in the context of normal epicardial coronary arteries on angiography. Its primary cause is unknown, but factors apparently involved are endothelial dysfunction and reduced flow in the tiny resistance blood vessels of the heart. Microvascular angina may be part of the pathophysiology of ischemic heart disease.

Refractory angina is a chronic condition (≥3 months in duration) in which angina (1) occurs in the context of coronary artery disease (CAD), (2) cannot be controlled by a combination of optimal medical therapy, angioplasty, or bypass surgery, and (3) in which reversible myocardial ischemia has been clinically established to be the cause of the symptoms.

### EXAMPLES

### Example 1

Wet granulated formulations containing 50 mg or 200 mg free base equivalent of CV-8972 were prepared according to the composition shown in Table 1 for the 200 mg.

TABLE 1

| | | Active MR Tablet | | Placebo MR Tablet | |
|---|---|---|---|---|---|
| | | | % | | % |
| Material | Grade | mg/tab | Composition | mg/tab | Composition |
| API | CV-8972 | 261.30^{1,2} | 47.90 | — | — |
| Mannitol USP/NF | Mannogem EZ | 106.04³ | 18.89 | 507.30 | 89.0% |
| Hypromellose | Methocel E5 LV | 5.54 | 1.01 | — | — |
| USP/NF | | | | | |
| Hypromellose | Methocel K100M | 166.05 | 30.20 | — | — |
| USP/NF | | | | | |
| Pre-gelatinized | Starch 1500 | — | — | 57.0 | 10% |
| Starch USP/NF | (Partially Pre- | | | | |
| | gelatinized Maize | | | | |
| | Starch 2001) | | | | |
| Magnesium | Vegetable grade | 11.07 | 2.01 | 5.7 | 1.0% |
| Stearate NF, BP, JP | | | | | |

| | | | | |
|---|---|---|---|---|
| Totals | 550.00⁴ | 100.00 | 570.00⁴ | 100.0 |

¹CV-8972 is the •3HCl•1H₂O salt of the free base (Mol. wt. = 542.86 g/mole); 261.30 mg salt equivalent to 200 mg of free base CV-8972
²Dosed as free base (Mol. Wt. = 415.49 g/mole)-ratio of Mol. Wts. = 1.3066
³adjusted for potency of each API lot
⁴Total tablet fill weight may vary ±5%

These wet granulation formulations are based on the previous direct compression, blended powders, which were filled into a single punch by hand and compressed individually. The two sets of formulations differ very slightly in composition to allow for the switch to wet granulation, but are identical in terms of specifications and performance.

Owing to the high amount of drug substance (API) present in the tablets (approximately 50%) and its potential impact on the tablet characteristics, it was considered prudent to switch from direct compression to wet granulation in order to provide some better control over the flow, hardness, and other characteristics. Formulation design and scale-up experiments confirmed that this was a justified decision, as blending, powder flow, and API bulk density variability proved problematic in providing a suitable direct compression formulation. (Further work is planned to evaluate a dry-granulated process for commercial development in order to replace the current wet granulation process to better facilitate scale-up and high throughput manufacturing). The 200 mg MR 8-hour release tablet is designed to be taken twice daily (BID) by patients.

The form of CV-8972 used for formulations has the following structure and properties:

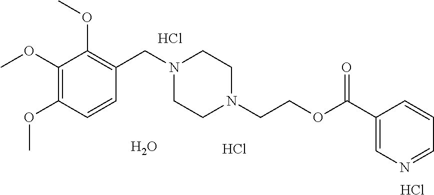 [SMILES: COc1ccc(CN2CCN(CCOC(=O)c3cccnc3)CC2)c(OC)c1OC ; O ; Cl ; Cl ; Cl]

Chemical name: 2-{4-[(2,3,4-trimethoxyphenyl)methyl]piperazin-1-yl}ethyl pyridine-3-carboxylate trihydrochloride monohydrate
Molecular Formula: C₂₂H₃₂N₃O₅Cl₃. 1H₂O
Molecular Weights: 542.86 g/mole (3HCl.1H₂O)
Associated Mol. Wts: 524.86 g/mole (3HCl anhydrous); 415.49 g/mole (free base, anhydrous)
pKa's (estimated): 4.85 (nicotinate); ˜5.50 (piperazine); ˜7.99 (piperazine)
cLog P (free base): 1.26-1.63
Melting Point: From DSC measurement on lot 289-MBA-33 (Tox.Lot):
Sharp melt at 221° C. preceded by broad band from 208° C. (DSC)
Loses majority of the monohydrate water between 100-127° C.
Calculated Melting Point=218.41° C.
Enthalpy of Fusion=−298 J/g

This molecule is a hybrid NCE which undergoes hydrolytic cleavage and metabolism as it is absorbed orally to produce CV-8814, Nicotinic acid, trimetazidine (TMZ), and potentially other metabolites systemically.

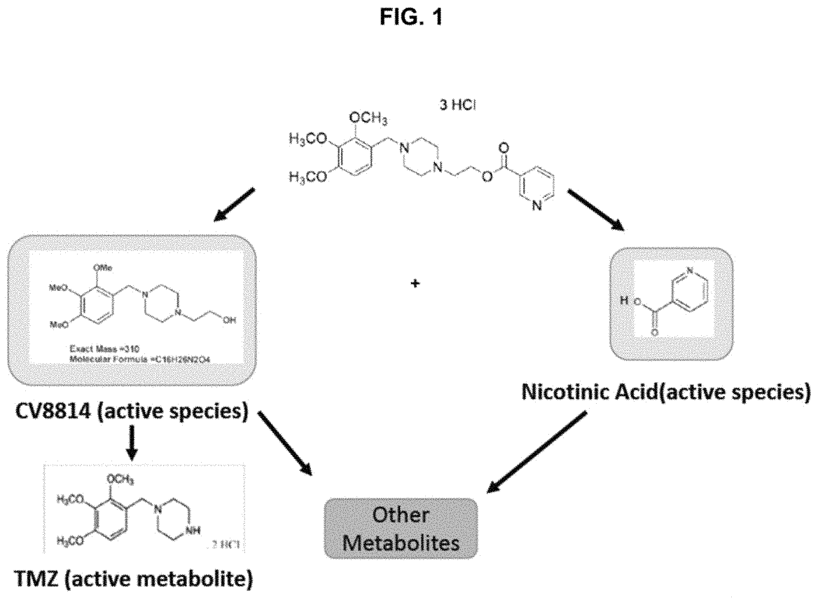

FIG. 1 is a schematic of the pathways of hydrolysis and metabolism of CV-8972. Note that there are other potential routes of metabolism not yet evaluated in humans.

CV-8972 as a salt is relatively insoluble in most organic solvents, but is much more so as the free base. However, as the most basic, estimated pKa is about 7.9, the molecule always exists as a charged ion in the physiological pH range of 1-8.

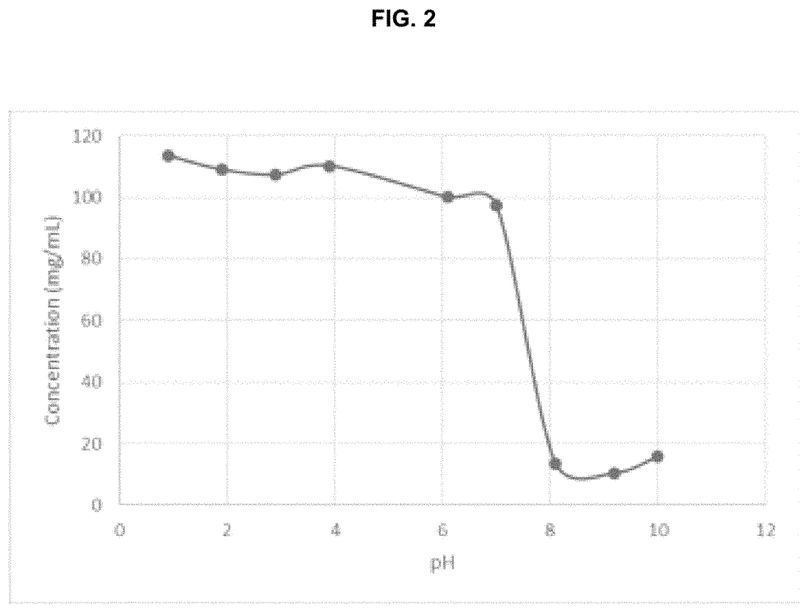

FIG. 2 is graph of the aqueous solubility of CV-8972 as a function of pH generated via dynamic methods due to instability. The acute drop in solubility between pH 6.1-9.2 suggests that dissolution kinetics could dramatically change on passage from gastric (pH 1-2) to the intestinal fluids (pH 6.5-8). However, given that the saturated base solubility is 5-10 mg/mL, a dose of 200 mg base is likely to be dissolving under sink conditions throughout the GI tract.

The pH stability profile is summarized and shown in Table 2 for a 1 mg/mL solution of CV-8972 at 23° C.

TABLE 2

| | | % lost | Amt. | | | | |
|---|---|---|---|---|---|---|---|
| | | in 25 | remaining | 1st order | T_{1/2} | T10% | T5% |
| pH | Medium | hrs | (mg/ml) | K_{obs} (hr − 1) | (hrs.) | (hrs.) | (hrs.) |
| 2.74 | Water | 12.1 | 0.879 | 0.00516 | 134.3 | 20.4 | 9.9 |
| 3.52 | 20 mM Phos | 6.6 | 0.934 | 0.00273 | 253.7 | 38.6 | 18.8 |
| 5.05 | 20 mM | 2.6 | 0.974 | 0.00105 | 657.5 | 100.0 | 48.7 |
| | Am. Acetate | | | | | | |
| 5.64 | 20 mM Phos | 6.0 | 0.94 | 0.00248 | 279.9 | 42.6 | 20.7 |
| 6.5 | 20 mM Phos | 24.4 | 0.756 | 0.01119 | 61.9 | 9.4 | 4.6 |
| 6.88 | 20 mM Phos | 40.3 | 0.597 | 0.02064 | 33.6 | 5.1 | 2.5 |

The hydrolysis proceeds via the first step shown in FIG. 1, as Nicotinic acid and CV-8814 are the only decomposition products generated. The optimal pH for solution stability appears to be around pH 5-5.1 for a 1 mg/mL solution with acid-catalyzed hydrolysis increasing at pH values less than 5.0 and base-catalyzed hydrolysis increasing above pH 5.0.

The excipients used in the formulation are standard for an erodible, hydrogel tablet to create a modified release product. As the CV-1018972 salt is very soluble, Hypromellose polymers of different molecular weights and degrees of cross-linking are used to hydrate gel and slowly release the soluble drug. Mannitol is included as a soluble sugar that balances the ingress of water into the formulation and Magnesium Stearate is used as a lubricant. Extensive screening of drug stability with a large array of excipients was previously performed, in addition to detailed stability evaluation of the drug in prototypical excipient blends and formulations.

### Example 2

Pilot and clinical tablet lots were manufactured by hand by direct blending and compression to support Phase 1 SAD/MAD studies at the ICU pharmacy. The formulations used in these evaluations are shown in Table 3.

TABLE 3

| | | Composition (mg/tab) | | | |
|---|---|---|---|---|---|
| | | | | 50 mg² − | |
| | | 50 mg² − | 200 mg² − | 8 Hr | 200 mg² − |
| Material | Grade | 4 Hr Release | 4 Hr Release | Release | 8 Hr Release |
| API | CV-89721^{1,} | 65.87 | 263.47 | 65.87 | 263.47 |
| Mannitol | Mannogem EZ³ | 360.33 | 135.05 | 304.87 | 107.27 |
| USP/NF | | | | | |
| Hypromellose | Methocel E5 LV | 5.53 | 5.53 | 5.65 | 5.65 |
| USP/NF | | | | | |
| Hypromellose | Methocel K4M | 110.70 | — | — | — |
| USP/NF | | | | | |
| Hypromellose | Methocel Kl00M | — | | 166.05 | 166.05 |
| USP/NF | | | | | |
| Hypromellose | Methocel K100 | | 138.37 | | |
| USP/NF | LV | | | | |
| Magnesium | Vegetable grade | 11.07 | 11.07 | 11.07 | 11.07 |
| Stearate | | | | | |
| USP/NF | | | | | |

| | | | | |
|---|---|---|---|---|
| Total⁴ | 553.50 | 553.50 | 553.51 | 553.51 |

¹CV-8972 is the •3HCl•1H₂O salt of the free base (Mol. wt. = 542.86 g/mole);
²Dosed as free base (Mol. Wt. = 415.49 g/mole)-ratio of Mol. Wts. = 1.3066
³adjusted for potency of each API lot
⁴Total tablet fill weight may vary ±5%

The formulations used for the studies were direct compression, blended powders filled into a single punch by hand and compressed individually at 50 mg and 200 mg free base equivalent of CV-8972 according to the composition shown in Table 3.

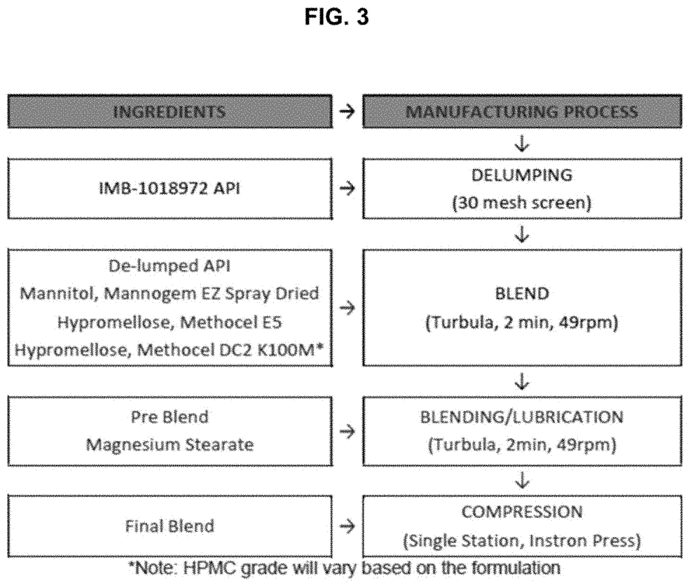

FIG. 3 is a process flow diagram for manufacture of the CV-8972 prototype dry compression MR tablet formulations.

The 8-hour MR release tablets were chosen for further studies and only at the doses of 200 mg and Placebo. The compositions are shown in Table 4.

TABLE 4

| | | | |
|---|---|---|---|
| % Composition | mg/Tablet | % Composition | mg/Tablet |

| | | |
|---|---|---|
| | 50 mg 8 Hr MR Tablet ¹ | 200 mg 8 Hr MR Tablet ¹ |
| Component | (lot #A0012007) | (lot #A0012017) |

High Shear Intra- Granular Components

| | | | | |
|---|---|---|---|---|
| CV-8972 tris HCl | 11.90% | 65.45 | 47.50%² | 261.25² |
| monohydrate(API) | | | | |
| Mannitol EZ (Diluent) | 55.10% | 303.05 | 19.30%² | 106.15² |
| HPMC E5 premium | 1.00% | 5.5 | 1.00% | 5.5 |
| LV(Binder) | | | | |

Extra Granular Components

| | | | | |
|---|---|---|---|---|
| HPMC K100M Premium DC2 | 30.00% | 165 | 30.20% | 166.1 |
| (Ext. Release polymer) | | | | |
| Magnesium stearate | 2.00% | 11 | 2.00% | 11.000 |
| (Lubricant) | | | | |
| TOTAL | 100.00% | 550.00 | 100.00% | 550.00 |

¹ Based on 550 mg total tablet weight and 100% purity of API
²The amounts of CV-8972 and Mannitol EZ are adjusted for potency of the API lot being used

The pilot demonstration lots were manufactured at the 5 kg level total batch size to evaluate performance with scale-up and equipment train compatibility for future GMP lots.

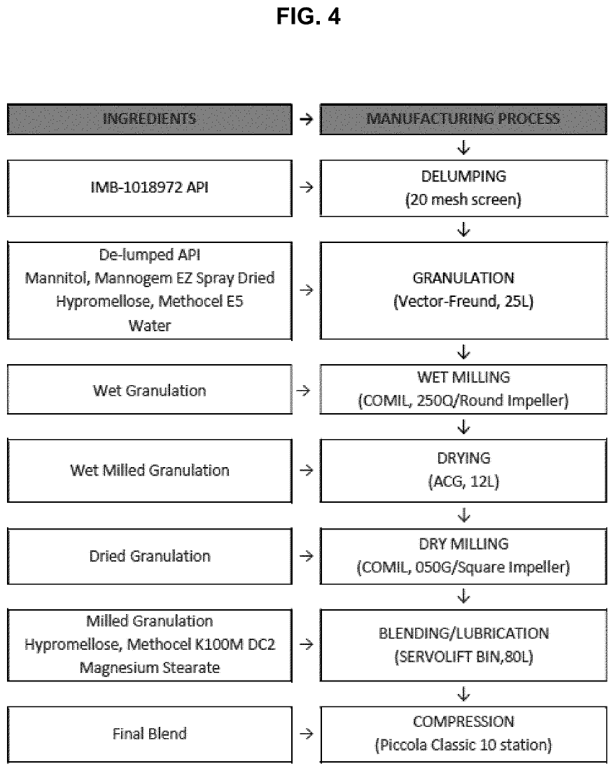

FIG. 4 is a process flow diagram for manufacture of the CV-8972 200 and 50 mg wet granulated, MR Tablet formulations used for pilot lots. The wet granulation was adopted with minor changes in composition as described in Table 1 and scaled up for manufacture of clinical supply lots each of 43,000 tablets approximately or −24 kg in total batch size.

A summary of the wet granulation process used for the manufacture of clinical supplies is provided here.
Screen the API thorough a 20 mesh screen.
Dispense the API, Mannitol, and Methocel E5 in four equal sections comprising the granulation sub-lots into an HPDE wide mouth drum.
Load each sub-lot of dry blend into a 25 L High Shear granulator and mix at Impeller speed 150 rpm, Chopper speed 360 rpm for 1 minute.
Spray 405 g of Purified Water USP via peristatic pump into the mixer and granulate using IMPELLER 150 RPM/CHOPPER 1500 RPM.
Continue to granulate until no dry powder remains and granules have begun to form at IMPELLER 65 RPM/CHOPPER 800 RPM.
Discharge wet granulation from the high shear granulator via a CoMill (Screen-250; Impellor Round; mill speed 2500 rpm) into a 12 L fluid bed drier bowl; dry to maintain adequate fluidization and a target product temperature of NLT 40° C. (inlet 60° C.; 110 Nm³/h volume throughput).
Determine % LOD at set times based on process experiments during scale up. The target LOD is NMT 2.0% at a target LOD temperature of 85° C.
Discharge the dry granulation and CoMill (screen −50; Impeller-square; 2500 rpm).
Repeat above steps three more times with other sublots and combine the granulations to determine the final weight; sieve through a 20-mesh screen; calculate the amount of Methocel® K100M Premium DC2 and Magnesium Stearate, sieve and add to the granules in the method referenced in steps 15-18.
Blend in an 80 L bin for 5 minutes at 15 rpm and tablet directly using pre-calibrated settings for tablet weight and hardness using 25 tablets.
   Theoretical weight=550 mg (API potency to be salt-factor adjusted)
   Mean weight 25 tablets=95-105% of theory
   Individual weight range=90-110% of theory
   Tablet hardness=15 kp (target); NLT 11 kp
Sample for assay and store bulk tablets in Poly Bags and 20 L HDPE drum.
Package from bulk tablet drum into patient bottles following full quality release and review. Package into 60 cc HDPE containers with a child-resistant caps at room temperature and place on stability storage conditions of 5° C., 25° C./60% RH, or 40° C./75% RH using added desiccant.

The following tests are applied to the release of the tablets:
Tablet Appearance
Identity, Assay, Purity in terms of CV-8814, Unknown and Total Impurities
Content Uniformity
Dissolution Release Testing
Residual Moisture
Tablet Hardness
Microbial Testing

The dissolution profiles for 50 mg and 200 mg MR tablet lots evaluated in the SAD/MAD studies and other pilot lots have been collected in 0.1N HCl. They achieve >80% dissolved in either 4 hours or 8 hours, depending on the formulation.

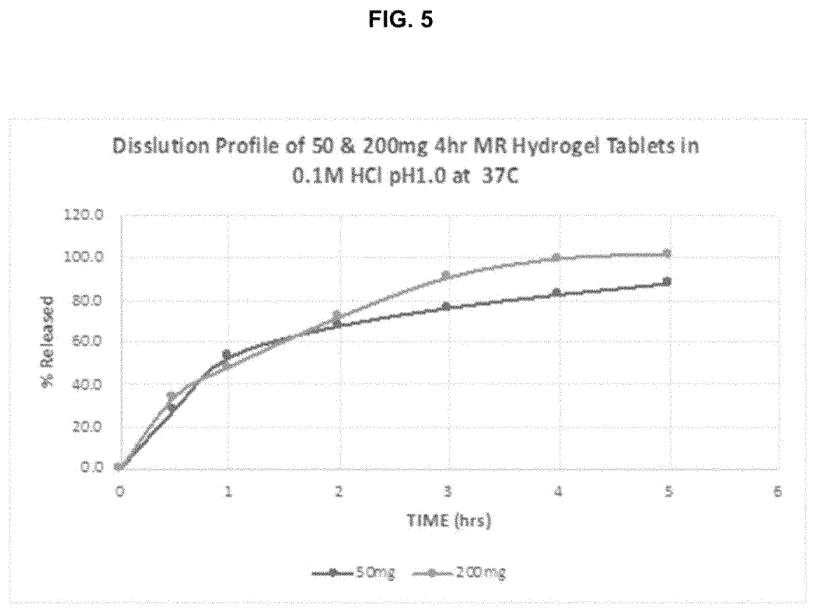

FIG. 5 is graph showing the dissolution profile of 50 mg and 200 mg 4-hour modified release hydrogel tablets in 0.1 M HCl pH 1.0 at 37° C.

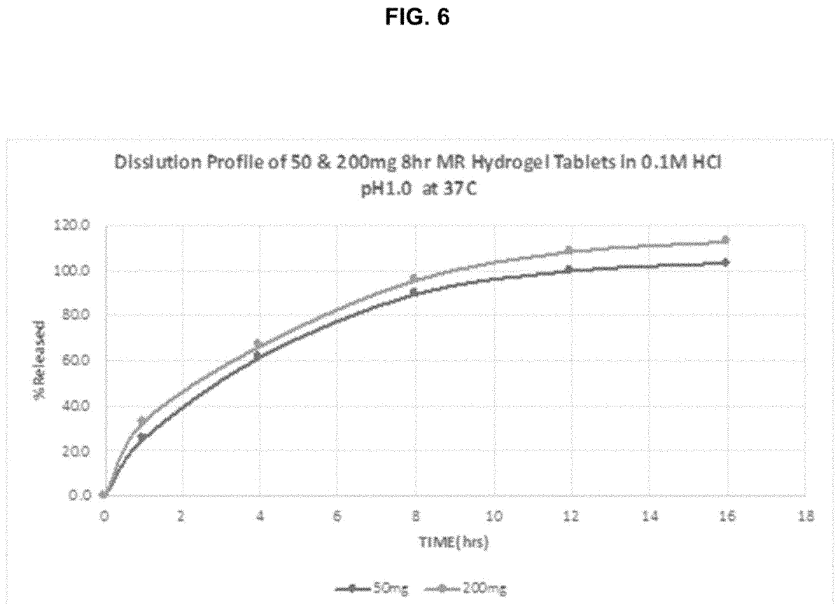

FIG. 6 is graph showing the dissolution profile of 50 mg and 200 mg 8-hour modified release hydrogel tablets in 0.1 M HCl pH 1.0 at 37° C.

Based on these data and the PK profiles obtained, tentative dissolution specifications and a validated dissolution assay were established against which release and stability samples have been evaluated.

Extensive stability evaluation of the 200 mg 8-hour MR tablets to be used at both 25° C./60% RH and 40° C./75% RH has demonstrated remarkable consistency of release when stored either with or without desiccant in the containers.

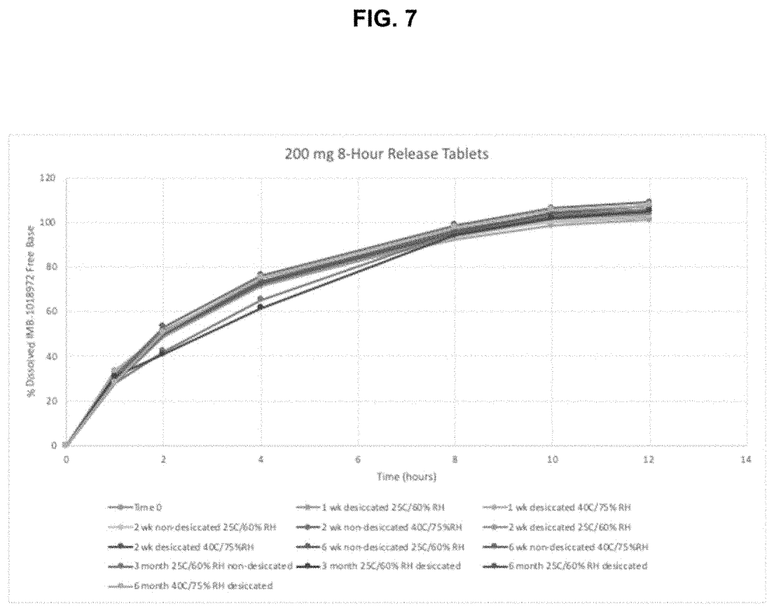

FIG. 7 is graph showing the dissolution profiles of 200 mg 8-hour modified release hydrogel tablets in 0.1 M HCl pH 1.0 at 37° C. following storage under indicated conditions. Tablet were stored at either 25° C./60% RH or 40° C./75% RH for 1 week, 2 weeks, 6 weeks, 3 months, or 6 months, with or without desiccant.

The specifications have been designed to control the combination of initial surface burst of release and the longer term release via matrix erosion to ensure consistent oral delivery. Table 5 shows the proposed dissolution test specifications and the range of means obtained over the ongoing 6-month pilot lot stability study.

TABLE 5

| | | Range (Means of N = 6) | |
|---|---|---|---|
| TIME | Spec. for Release | Lowest | Highest |
| 1 hour | ≤50% | 28.0 | 35.3 |
| 2 hours | 30-65% | 42.2 | 53.3 |
| 8 hours | ≥80% | 92.4 | 98.9 |

In addition, the dissolution data at T=0 for the three clinical lots made under cGMP conditions are shown in Table 6.

TABLE 6

| Lot | 1 hour | 2 hour | 4 hour | 8 hour | 10 hour | 12 hour |
|---|---|---|---|---|---|---|
| DP-APS19-164 | 34.8 | 51.2 | 71.9 | 91.7 | 95.9 | 98.1 |
| DP-APS19-165 | 34.5 | 51.3 | 73.1 | 92.8 | 96.3 | 98.5 |
| DP-APS19-166 | 34.4 | 51.4 | 73.1 | 92.5 | 95.9 | 97.6 |

The overall conclusions from the 200 mg 8-hour MR dissolution studies are:
that the release formulation is extremely consistent and robust;
from a stability perspective, there is little variability seen even under accelerated conditions;
that the formulation performance is reproducible when scaled up and run multiple times.

### Example 3

As can be seen from the previous examples, CV-8972 is very soluble as a salt and a free base with a minimal solubility of around 10 mg/mL as the free base and >100 mg/mL as the salt. Thus, a 250 mL volume of neutral pH aqueous solution can dissolve up to 2.5 g of CV-8972, which is significantly higher by 5-10× that of the potential maximal daily dose of 250-500 mg.

The calculated cLog P for CV-8972 is around 1.5 and in vitro CaCo2 experiments show permeability (Pₐₚₚ) to be much greater than 1×10′ cm/s at around 2.9×10⁻⁵ cm/sec with little evidence of efflux. By comparison, in the same study CV-8814 demonstrated similar behavior.

CV-8972 can therefore be considered as BCS Class 1 per se, but the pre-systemic metabolism to CV-8814 and possibly other metabolites may complicate and impact the actual biopharmaceutical absorption profile found in humans. CV-8972 in dog is estimated to have a >50% bioavailability when given as solution versus IV CV-8814.

The solution stability of CV-8972 was evaluated in multiple media currently used to simulate dissolution and stability conditions in the various sections of the GI tract. As CV-8972 will be subject to hydrolysis as it is dissolving in the GI tract, it will be important to understand these kinetics in terms of designing instant and controlled-release formulations. HPLC methods were used to perform these assessments. A summary of the decomposition kinetics vs. media for CV-8972 at 37° C. and 0.2 mg/mL is shown in Table 7.

TABLE 7

| Media | pH | Est K_{obs} (hrs⁻¹)⁴ | T_{1/2} (hrs) | T_{1/2} (mins) | Components (mM)³ |
|---|---|---|---|---|---|
| 0.1M HCl | 1.0 | 0.221 | 3.13 | 188 | No surfactants |
| FaSSGF¹ | 1.6 | 0.0143 | 48.5 | 2912 | NaT(3); Lec(0.2) |
| FaSSIF¹ | 6.5 | 0.05124 | 13.5 | 811 | NaT(3); Lec(0.2) |
| FeSSIF¹ | 5.8 | 0.00696 | 99.6 | 5974 | NaT(10); |
| | | | | | Lec(2);GMO(5);NaO(0.8) |
| BiCarb adj² | 6-8.2 | 0.455994 | 1.52 | 91.2 | No surfactants |

¹As defined by Jantratid & Dressman;
²0.1M HCl solution neutralized with Sodium Bicarbonate soln. (>1M);
³Surfactant components Sodium Taurocholoate (NaT); Lecithin (Lec);
Glyceryl Mono-oleate (GMO); Sodium Oleate (NaO); No enzymes were present.
⁴K_{obs} is the calculated first order rate constant for the hydrolysis of CV-8972

Points to note about the data in Table 7 are:
There is clearly more complex behavior/interaction of CV-8972 with the components of FaSSGF, FaSSIF, and FeSSIF that cannot all be explained by pH differences.
The presence of surfactants such as bile acids, lecithins, or Glyceryl mono-oleate appears to “protect” CV-8972 or slow down its hydrolysis rate, probably via complexation.
While the above may suggest a potentially beneficial food effect in vivo, little impact of fed versus fasted was actually observed in humans for the 200 mg, 8-hour, MR tablet (see below in Section 5.1.2).

In summary, there may be some protective effect of the components of the FaSSGF, FaSSIF, and FeSSIF, particularly when compared to stability in 0.1N HCl or bicarbonate or phosphate buffer at pH 6-8. On the positive side, the fastest decomposition rate still has a T_{1/2} of 1.5 hours which should provide enough residence time for release and absorption in the upper GI tract.

Although stability in 0.1N HCl at 37° C. appears low, it arguably provides the harshest test of the in vitro release and resistance to hydrolysis for a dissolution test. CV-8972 is considered to be stable enough to use this condition via pH stabilization and rapid assay accounting for parent and CV-8814. There was found to be no difference in dissolution rate between 0.1N HCl, water, and pH 6.8 phosphate buffer. Dissolution in pH 5 acetate buffer, FaSSGF, FaSSIF, and FeSSIF was found to be analytically complex due to interferences with the release and analytical measurement by HPLC. Analytical methodology will be developed to resolve this in parallel with the ongoing evolution of IVIVC/IVIVR data.

Additional and extensive in vitro stability work has been carried out across species to evaluate the impact of GI tract, liver tissues, and plasma on half-life as measured by in vitro clearance of CV-8972 in various biological media. It suggests that CV-8972 is likely to be relatively unstable in the GI tract (per its design), but may be stable enough at the intestinal brush border and the enzymes within it to allow sufficient absorption to occur in intact form.

To enable the evaluation and choice of MR formulations in humans, PK studies in dogs were performed under fasted conditions in order to determine how well the in vitro and in vivo release profiles correlated.

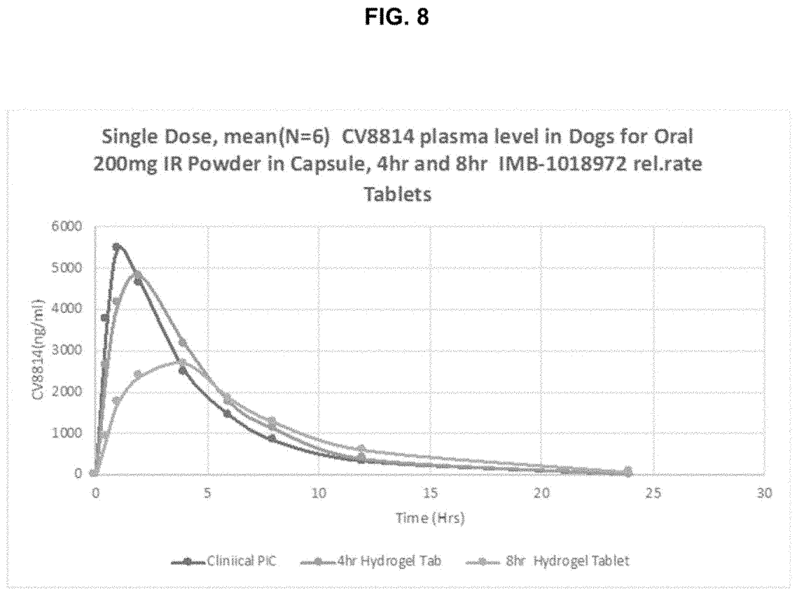

FIG. 8 is a graph showing CV-8814 plasma levels in dogs following oral dosing of 200 mg (free base equivalent) of CV-8972 under fasted conditions. CV-8972 was provided as an instant release powder in a capsule, (b) a 4-hour release rate modified release tablet, or an 8-hour release rate modified release tablet.

Table 8 summarizes the comparison of the calculated in vitro release rate constant (K_{d} hrs⁻¹) and time to release 90% (T_{90%} hrs) with the corresponding in vivo absorption rate constant (Kₐ hrs⁻¹) and time to absorb 90% of the dose (T_{90%} hrs) as calculated via the Wagner-Nelson technique².

TABLE 8

| | K_{d} (hrs −1) Disso | Kₐ (hrs −1) PK | T₉₀ Disso (Hrs) | T₉₀ Abs (Hrs) |
|---|---|---|---|---|
| Formulation | In Vitro | In Vivo | In Vitro | In Vivo |
| 200 mg IR | 5.6 | 3.1 | 0.4 | 0.7 |
| 200 mg 4 hr Hydrogel | 0.7 | 2.0 | 3.4 | 1.2 |
| 200 mg 8 hr Hydrogel | 0.3 | 0.6 | 7.7 | 3.6 |

In general, the in vivo data seemed to show faster absorption than is predicted by in vitro data by a factor of ˜2× for the 4-hour and 8-hour release tablets. Due to metabolic differences between dog and human, the predictive value of these data to humans may be limited, but the estimation of absorption kinetic parameters is considered useful at this stage of development. This approach will be used similarly in humans to de-convolute the absorption PK curves and correlate the in vivo and in vitro release rates.

Recent data obtained from humans in SAD/MAD studies, including fed/fasting, are currently being evaluated; preliminary evaluation shows similar behavior of the 4-hour and 8-hour release tablets at 50 mg and 200 mg to that seen in dogs, but needs more in-depth analysis to make a valid comparison. Little or no food effect on human PK seems to have been observed which appears to be a beneficial outcome of this study with respect to dosing of patients.

Some modelling has already been done with respect to estimating the Cₘᵢₙ versus Cₘₐₓ levels at steady-state following 200 mg 8-hour MR tablet dosing. The impact and benefit of BID over QD dosing for this formulation is clearly seen for CV-8814, showing a 10× decrease in Cₘₐₓ/Cₘᵢₙ ratio of BID versus QD dosing; the comparison is less so for TMZ (ratio of ˜2× reduction) and the combined levels (ratio of ˜3× reduction). Again, this supports and confirms the benefit of BID dosing for this formulation.

Further evaluation is being undertaken to assess the initial IV/IVC read-out in humans covering both active species CV-8814 and Trimetazidine (TMZ).

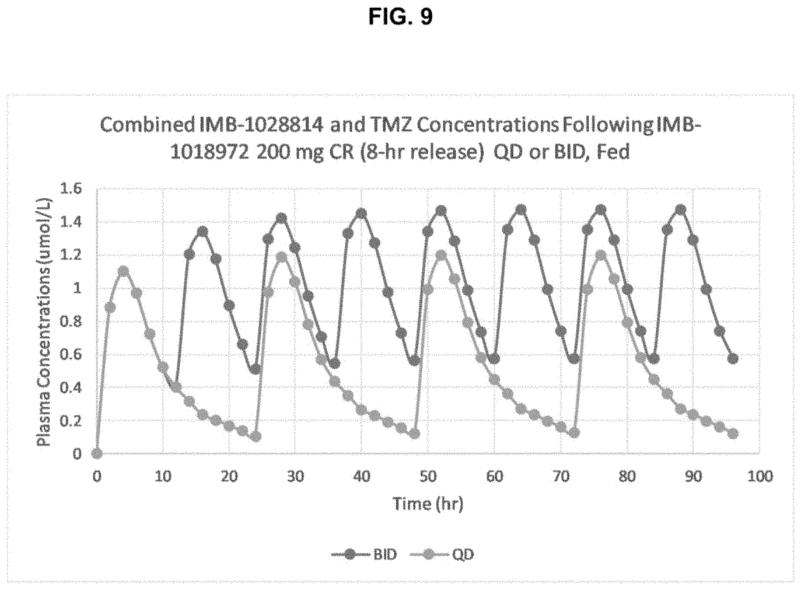

FIG. 9 is a graph showing the simulated combined plasma levels of CV-8814 and trimetazidine projected in humans following oral dosing 200 mg IMB-1018972 MR tablets as 8-hour release tablets under MAD dosing (QD and BID) and fed conditions.

Table 9 shows projected Cₘₐₓ, Cₘᵢₙ, and Cₘₐₓ/Cₘᵢₙ ratio for of CV-8814, trimetazidine, and both in humans following oral dosing 200 mg IMB-1018972 MR tablets as 8-hour release tablets under MAD dosing (QD and BID) and fed conditions.

TABLE 9

| | CV-8972 | Cₘₐₓ | Cₘᵢₙ | Cₘₐₓ/Cₘᵢₙ |
|---|---|---|---|---|
| Compound | Dose Frequency | (μmol/ml) | (μmol/ml) | ratio |
| CV-8814 | BID | 0.825 | 0.169 | 4.9 |
| TMZ | BID | 0.646 | 0.407 | 1.6 |
| CV-8814 + | BID | 1.471 | 0.576 | 2.6 |
| TMZ | | | | |
| CV-8814 | QD | 0.758 | 0.015 | 51.1 |
| TMZ | QD | 0.439 | 0.113 | 3.9 |
| CV-8814 + | QD | 1.197 | 0.127 | 9.4 |
| TMZ | | | | |

### Example 4

### Study Design

A Phase 1 first-in-human, randomized, double-blind, study to investigate the safety, tolerability, and pharmacokinetics (including food effect) or IMB-1018972 in healthy subjects.

Objectives

The primary objective is to assess the safety and tolerability of single oral doses of modified-release (MR) formulations of IMB-1018972, and multiple oral doses of the 200 mg 8-hour MR formulation of IMB-1018972 in healthy subjects.

Secondary objectives include: To assess the pharmacokinetic (PK) profile of single oral doses of MR formulations of IMB-1018972, and multiple oral doses of the 200 mg 8-hour MR formulation of IMB-1018972 in healthy subjects; To assess the absorption and PK profile of the 200 mg 8-hour MR formulation of IMB-1018972 following multiple oral doses taken with food in healthy subjects; and To evaluate the safety and tolerability of the 200 mg 8-hour MR formulation of IMB-1018972 following multiple oral doses taken with food in healthy subjects.

Design and Treatments

This was a double-blind, randomized, study consisting of single-dose and multiple-dose MR parts to assess the safety, tolerability, and PK single oral doses of a MR formulation of trimetazidine, single oral doses of MR formulations of IMB-1018972, and multiple oral doses of the 200 mg 8-hour MR formulation of IMB-1018972.

Single-Dose MR Part

In the single-dose MR part, 1 group of 12 healthy subjects (all on active drug) was included. The subjects received a single oral dose of 1 of 4 MR formulations of IMB-1018972 under fasted conditions (an overnight fast of at least 10 hours) on Days 1, 4, 7, and 10 in a fixed order that was the same for all subjects. The MR formulation of IMB-1018972 to be administered on Day 13 under fed conditions was 1 of the 4 MR formulations administered on Days 1, 4, 7, and 10 under fasted conditions. The formulation chosen for administration on Day 13 was the 200 mg 8-hour MR formulation as determined by the Sponsor based on the available safety, tolerability, and PK results of the 4 MR formulations.

The following treatments were administered in the single-dose MR part:
Day 1: single oral dose of 50 mg 8-hour MR formulation of IMB-1018972 (n=12) under fasted conditions
Day 4: single oral dose of 50 mg 4-hour MR formulation of IMB-1018972 (n=12) under fasted conditions
Day 7: single oral dose of 200 mg 8-hour MR formulation of IMB-1018972 (n=12) under fasted conditions
Day 10: single oral dose of 200 mg 4-hour MR formulation of IMB-1018972 (n=12) under fasted conditions
Day 13: single oral dose of 200 mg 8-hour MR formulation of IMB-1018972 (n=12) under fed conditions

### Multiple-Dose MR Part

In the multiple-dose MR part, 1 group of 12 healthy subjects (all on active drug) was included. Subjects received multiple oral doses of the 200 mg 8-hour MR formulation of IMB-1018972 q12h under fed conditions for 5 consecutive days; on Day 5 only a single morning dose was administered.

Study Schedule
Screening: Between Day −35 and Day −1 (admission)
Confinement period: Single-dose MR part: 1 period in the clinic from Day −1 (admission) to approximately 72 hours after study drug administration on Day 13 (Day 16) Multiple-dose MR part: 1 period in the clinic from Day −1 (admission) to approximately 48 hours after study drug administration on Day 5 (Day 7)
Follow-up: Single-dose MR part: 7 to 14 days after the last PK blood sample (between Day 23 and Day 30); Multiple-dose MR part: 6 to 8 days after the last PK blood sample (Day 14±1 day)

Subjects
Single-dose MR part: 12 healthy male or female subjects; for this group, all efforts were made to have a ratio of 50:50 for male and female subjects, but at minimum at least 4 subjects of each gender were dosed
Multiple-dose MR part: 12 healthy male or female subjects; for this group, all efforts were made to have a ratio of 50:50 for male and female subjects, but at minimum at least 4 subjects of each gender were dosed

Main Criteria for Inclusion
Age: 18 years to 65 years, inclusive, at screening
Body mass index (BMI): 18.0 kg/m² to 32.0 kg/m², inclusive
Status: Healthy subjects

Study Drug

### Active Medication

Drug product: IMB-1018972
Activity: Fatty acid oxidation inhibitor
In development for: Ischemic cardiovascular disease
Strength: 50 mg MR formulation and 200 mg MR formulation with 4-hour and 8-hour dissolution profile (based on free base)
Dosage form: oral MR tablet(s) to be used in the MR parts
Manufacturer: Pharmacy at PRA
Batch number: 2479-1810-00441 (drug substance)

### Active Medication

Drug product: Vastarel MR (trimetazidine dihydrochloride)
Activity: Fatty acid oxidation inhibitor
In development for: Angina pectoris
Strength: 35 mg
Dosage form: Oral modified-release tablet
Manufacturer: Servier Research & Pharmaceuticals (Pakistan) (Pvt.) Ltd.
Batch number: 273782 (drug product)

### Variables

Safety: Adverse events, clinical laboratory, vital signs, 12-lead electrocardiogram, continuous cardiac monitoring (telemetry), and physical examination
Pharmacokinetics: Plasma concentrations of IMB-1018972, IMB-1028814, and trimetazidine Urine concentrations of IMB-1018972, IMB-1028814, and trimetazidine single-dose MR part: Cₘₐₓ, tₘₐₓ, AUC₀₋ₜ, AUC_{0-inf}, % AUCₑₓₜᵣₐ, kₑ₁, t_{1/2}, CL/F (IMB-1028814 only), and V_{z}/F (IMB-1028814 only). Day 1 of multiple-dose MR part: Cₘₐₓ, tₘₐₓ, and AUC₀₋ₜ. and Day 5 of multiple-dose MR part: Cₘₐₓ, tₘₐₓ, Cₘᵢₙ, kₑ₁, t_{1/2}, AUC_{0-T}, CL_{SS}/F (IMB-1028814 only), V_{z}/F (IMB-1028814 only), and Rae Urine PK parameters estimated using noncompartmental analysis, as appropriate: Aeᵤᵣᵢₙₑ, Feᵤᵣᵢₙₑ, and CL_{R}

Statistical Methods
Sample size calculation: For this FIH study, no prospective calculations of statistical power were made. The sample size was selected to provide information on safety, tolerability, and PK single doses of MR formulations of IMB-1018972, and multiple doses of the 200 mg 8-hour MR formulation of IMB-1018972, and is typical for a FIH study. Any p-values to be calculated according to the statistical analysis plan were interpreted in the perspective of the exploratory character of this study.
Safety parameters: Descriptive statistics
PK parameters: Descriptive statistics for all relevant PK parameters: n, mean, SD, minimum, median, maximum, geometric mean, and coefficient of variation; analysis of variance on Cₘₐₓ and AUC parameters to determine dose proportionality and FE

Results

### Subject Disposition

One subject of the single-dose MR part was withdrawn from the study due to a moderate treatment-emergent adverse event (TEAE) of alanine aminotransferase (ALT) increased (possibly related; up to 149 IU/L on Day 11) and did not receive the last single oral dose of 200 mg 8-hour MR formulation of IMB-1018972 under fed conditions on Day 13. None of these discontinued subjects were replaced. All 88 subjects were included in the PK and safety sets.

TABLE 10
Disposition of Subject

| | Number of subjects |
|---|---|
| Screened volunteers | 220 |
| Screening failures | 78 |
| Approved but not receiving study drug | 54 |
| Reserve | 24 |
| Group full | 9 |
| Personal reasons | 9 |
| Group cancelled | 8 |
| Rejected in clinic | 3 |
| Illness of volunteer | 1 |
| Subjects receiving at least 1 dose of | 88 |
| study drug | |
| Any dose of IMB-1018972 | 66 |
| Placebo dose | 14 |
| Trimetazidine dose | 8 |
| Discontinued subjects | |
| Adverse event | 2 |
| Withdrawal by subject | 1 |
| Completed subjects | 85 |

### Demographics

Single-Dose MR Part

Twelve subjects were included of whom 6 were female and 6 were male. Mean age was 32 years and mean BMI was 25.8 kg/m2. Individual age ranged between 19 and 62 years and individual BMI ranged between 21.5 and 31.0 kg/m2. Eleven subjects were of white race and 1 subject was Black or African American.

Multiple-Dose MR Part

Twelve subjects were included of whom 6 were female and 6 were male. Mean age was 45 years and mean BMI was 25.1 kg/m2. Individual age ranged between 24 and 64 years and individual BMI ranged between 20.0 and 29.2 kg/m2. Eleven subjects were of white race and 1 subject was Asian.

Safety

In the single-dose MR part, treatment with the 50 mg MR formulation and 200 mg MR formulation with 4-hour and 8-hour dissolution profile under fasted conditions, and subsequently, the 200 mg 8-hour MR formulation of IMB-1018972 under fed conditions was well tolerated by healthy male and female subjects, except for 1 subject in which ALT was elevated (up to 149 IU/L) after 2 single doses of 50 mg MR formulation and 2 doses of 200 mg MR formulation. No TEAEs of flushing were reported in the single-dose MR part.

In the multiple-dose MR part, 5-day treatment with multiple oral doses of the 200 mg 8-hour MR formulation of IMB-1018972 q12h under fed conditions was well tolerated by healthy male and female subjects. Flushing of mild severity was reported by 2 subjects who were post-menopausal females and 1 of whom had reported ongoing “hot flushes” as part of medical history. No subjects dropped out and no modification of the dose was needed due to the TEAEs of flushing.

Overall, no deaths were reported during the study. The majority of the reported TEAEs were transient and resolved without sequelae by follow-up. Most TEAEs were of mild severity and no severe TEAEs were reported during the study. TEAEs of moderate severity were the 5 TEAEs of flushing mentioned above and 1 TEAE each of restlessness, back pain, nausea, tonsillitis, post procedural hemorrhage, ALT increased, and influenza like illness. The moderate TEAE of ALT increased was reported by a subject of the single-dose MR part. This subject was withdrawn from the study as a result of this TEAE. The TEAE of ALT increased (up to 149 IU/L on Day 11) was considered by the Investigator to be possibly related to the study drug.

In the single-dose MR part, there was no clear difference between fasted and fed IMB-1018972 administration for the number and incidence of TEAEs.

The most frequently reported TEAEs during the study were of the system organ class vascular disorders (mainly TEAEs of flushing), general disorders and administration site conditions, nervous system disorders, gastrointestinal disorders, and musculoskeletal and connective tissue disorders.

The majority of the TEAEs reported during the study were considered by the Investigator not to be related to the study drug.

There were no findings of clinical relevance with respect to clinical laboratory, vital signs, 12-lead ECG, continuous cardiac monitoring (telemetry), or physical examination.

### Pharmacokinetics

All blood samples of subjects that received IMB-1018972 in this study were analyzed for IMB-1018972 in plasma, but IMB-1018972 could be measured in only few plasma samples. Therefore, the IMB-1018972 concentrations have only been listed and no descriptive statistics or concentration-time profiles have been presented in this clinical study report. In addition, no PK parameters have been calculated for plasma IMB-1018972. As a result, urine samples were not analyzed for IMB-1018972 concentrations.

Since the pharmacodynamic effect of IMB-1028814 and trimetazidine is the same, data are presented for IMB-1028814 and trimetazidine individually, as well as for the sum of IMB-1028814 and trimetazidine concentrations.

### Single-Dose MR Part

Following administration of both the 50-mg and 200-mg single oral fasted doses of IMB-1018972, tₘₐₓ for IMB-1028814 was earlier with the 8-hour MR formulation (2 hours for 50 mg and 200 mg IMB-1018972) than with the 4-hour MR formulation (5 hours for 50 mg IMB-1018972 and 3 hours for 200 mg IMB-1018972). Following administration of both the 50-mg and 200-mg single oral fasted doses of IMB-1018972, tₘₐₓ for trimetazidine was later with the 8-hour MR formulation (8 hours for 50 mg IMB-1018972 and 5 hours for 200 mg IMB-1018972) than with the 4-hour MR formulation (6 hours for 50 mg IMB-1018972 and 3 hours for 200 mg IMB-1018972). Following administration of both the 50-mg and 200-mg single oral fasted doses of IMB-1018972, tₘₐₓ for IMB-1028814+trimetazidine was similar for the 8-hour MR formulation (5 hours for 50 mg IMB-1018972 and 2.5 hours for 200 mg IMB-1018972) and the 4-hour MR formulation (5 hours for 50 mg IMB-1018972 and 3 hours for 200 mg IMB-1018972).

Following administration of the 50-mg and 200-mg single oral fasted doses of IMB-1018972, Cₘₐₓ for IMB-1028814 was 35% and 32% lower, respectively, Cₘₐₓ for trimetazidine was 20% and 24% lower, respectively, and Cₘₐₓ for IMB-1028814+trimetazidine was 21% and 34% lower, respectively, for the 8-hour MR formulation relative to the 4-hour MR formulation.

Following administration of the 50-mg single oral fasted dose of IMB-1018972, AUC₀₋ₜ for IMB-1028814 was 26% lower, AUC₀₋ₜ for trimetazidine was 12% lower, and AUC₀₋ₜ for IMB-1028814+trimetazidine was 18% lower after the 8-hour MR formulation than after the 4-hour MR formulation. Following the 200-mg single oral fasted dose of IMB-1018972, AUC₀₋ₜ for IMB-1028814 was 6% higher, AUC₀₋ₜ for trimetazidine was 4% higher, and AUC₀₋ₜ for IMB-1028814+trimetazidine was 5% higher after the 8-hour MR formulation than after the 4-hour MR formulation.

Following administration of the 50-mg MR formulation and 200 mg MR formulation with 4-hour and 8-hour dissolution profile under fasted conditions, geometric mean t½ ranged between 3.35 hours and 4.27 hours for IMB-1028814, between 8.11 hours and 9.35 hours for trimetazidine, and between 6.95 hours and 7.96 hours for IMB-1028814+trimetazidine. Thus, for each of the analytes, no difference was observed in t½ was between the 4 fasted treatments.

### Effect of Food

The possible effect of food on the PK of IMB-1028814 and trimetazidine was explored by comparing administration of single oral doses of 200 mg MR formulation of IMB-1018972 with 8-hour dissolution profile after an FDA-defined high-fat breakfast and under fasted conditions.

Median IMB-1028814 tₘₐₓ was reached at 3 hours postdose under both conditions. Median trimetazidine tₘₐₓ was reached at 5 hours postdose relative to 3 hours postdose under fasted conditions.

The effect of food of IMB-1028814 and trimetazidine was explored for Cₘₐₓ, AUC₀₋ₜ, and AUC_{0-inf}. No evidence for an effect of food was observed on the IMB-1028814 exposure parameters AUC₀₋ₜ and AUC_{0-inf} (both with an estimate of 1.16 and 90% CI ranging from 1.05 to 1.28). However, Cₘₐₓ was approximately 42% higher following administration of a single dose of 200 mg 8-hour MR IMB-1018972 after an FDA-defined high-fat breakfast relative to administration under fasted conditions (estimate of 1.42; 90% CI ranging from 1.24 to 1.63).

No evidence for an effect of food was observed on the trimetazidine exposure parameters Cₘₐₓ (estimate of 1.10; 90% CI ranging from 0.99 to 1.21), AUC₀₋ₜ (estimate of 0.99; 90% CI ranging from 0.91 to 1.09), and AUC_{0-inf} (estimate of 0.97; 90% CI ranging from 0.88 to 1.07) following administration of a single dose of 200 mg 8-hour MR IMB-1018972.

### Multiple-Dose MR Part

Following administration of the 200 mg 8-hour MR IMB-1018972 dose, median IMB-1028814 tₘₐₓ was 2 hours on Day 1 and Day 5, and median tₘₐₓ was 5.5 hours and 5 hours for trimetazidine on Day 1 and Day 5, respectively.

Based upon visual inspection of the geometric mean plasma concentration-time profiles and the geometric mean trough concentrations, it can be concluded that steady state for both IMB-1028814 and trimetazidine concentrations was reached by Day 5 following multiple dose administration of 200 mg 8-hour MR IMB-1018972.

Geometric mean R_{ac} for IMB-1028814, trimetazidine, and IMB-1028814+trimetazidine were 1.22, 2.28, and 1.66 on Day 5 relative to Day 1. This indicates minimal accumulation of IMB-1028814 in plasma, moderate accumulation of trimetazidine in plasma, and moderate accumulation of IMB-1028814+trimetazidine in plasma. The geometric mean half-life of the 200 mg 8-hour MR IMB-1018972 dose was 3.85 hours, 9.52 hours, and 8.64 hours for IMB-1028814, trimetazidine, and IMB-1028814+trimetazidine, respectively.

### Conclusions

### Safety

Overall, single and multiple doses of MR formulations, were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the MR formulations.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions, no clear difference between fasted and fed IMB-1018972 administration for the number and incidence of TEAEs was observed in the single-dose MR part.

### Pharmacokinetics

IMB-1018972 could be measured in only few plasma samples taken during this study.
When combining the single and multiple MR formulations of IMB-1018972 dose results under fasted and fed conditions, the initial hydrolysis of IMB-1018972 to IMB-1028814 and subsequent systemic bioavailability of IMB-1028814 was relatively rapid with median tₘₐₓ ranging between 0.5 hours and 5 hours postdose for IMB-1028814, and between 1.5 hours and 8 hours postdose for trimetazidine. Median tₘₐₓ did not increase with increasing IMB-1018972 dose
No evidence for an effect of food was observed on the IMB-1028814 exposure parameters AUC₀₋ₜ and AUC_{0-inf} following administration of a single dose of 200 mg 8-hour MR IMB-1018972. However, Cₘₐₓ was approximately 42% higher following administration of a single dose of 200 mg 8-hour MR IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 200 mg 8-hour MR IMB-1018972.
When combining the single and multiple IMB-1018972 dose results under fasted and fed conditions, the geometric mean t½ ranged between 2.5 hours and 4.5 hours for IMB-1028814, and between 6.5 hours and 9.5 hours for trimetazidine. Geometric mean t_{1/2} did not increase with increasing IMB-1018972 dose.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted as trimetazidine.
Within 48 hours following administration of a single oral dose of 35 mg trimetazidine, on average 54.47% of the dose was excreted in urine as trimetazidine.
Following 5 days of twice daily dosing with 200 mg 8-hour MR IMB-1018972 under fed conditions, no relevant accumulation of IMB-1028814 (Rac of 1.22) was observed, whereas accumulation of trimetazidine was moderate (Rac of 2.28).

### Overall

In view of the positive risk/benefit profile and the observed PK characteristics of the IMB-1018972 metabolites IMB-1028814 and trimetazidine in this FIH study, further clinical development of IMB-1018972 is warranted.

### Introduction

IMB-1018972 is an orally administered small molecule that is being developed as a treatment for ischemic cardiovascular disease and the associated abnormal cellular energetics. Potential indications include angina pectoris, heart failure, and peripheral vascular disease. IMB-1018972 is a new chemical entity (NCE) of the drug class partial fatty acid oxidation (pFOX) inhibitors that acts to preserve or enhance energy metabolism in cells exposed to hypoxia or ischemia. Other pFOX inhibitors include ranolazine (Ranexa), perhexiline, and trimetazidine. Glucose oxidation is a more efficient producer of adenosine triphosphate per oxygen molecule consumed compared to fatty acid oxidation.

IMB-1018972 undergoes hydrolysis after administration, and the hydrolysis products are nicotinic acid (also known as niacin or vitamin B3) and an inhibitor of 3-ketoacyl CoA thiolase (3-KAT) named IMB-1028814. In addition to IMB-1018972, IMB-1028814 has been studied and characterized extensively in nonclinical studies. IMB-1028814 undergoes further metabolism and 1 metabolite is trimetazidine, a drug marketed in Europe since 1987 for the treatment of angina pectoris.

The primary mechanism of action of IMB-1028814 is thought to be competitive inhibition of 3-KAT that results in the shift of substrate utilization in the myocardium from fatty acid oxidation to glucose oxidation. The delivery of nicotinic acid may serve to additionally enhance cellular energetics.

The nonclinical pharmacology and toxicology data collected at the time the CSP was finalized supported conducting clinical studies that administer IMB-1018972 for up to 4 weeks to assess its safety, tolerability, PK, and pharmacodynamics in humans.

Trimetazidine administered in this study is a drug marketed in Europe since 1978 for the treatment of angina pectoris.

Study Rationale

No clinical studies with IMB-1018972 had been performed prior to the study described in this CSR. Therefore, this first-in-human study (FIH), was conducted to assess the safety, tolerability, and PK of IMB-1018972 as a modified-release (MR) formulation.

During the study, a single-dose MR part was added assessing the safety, tolerability, and PK profile of single oral doses of newly developed MR formulations of IMB-1018972. These concerned 4 MR formulations: 50 mg and 200 mg dose strengths of IMB-1018972, each with a 4-hour dissolution profile and an 8-hour dissolution profile. The objectives of these MR formulations were two-fold: the first objective was to lower the Cₘₐₓ of IMB-1018972 and its subsequent metabolites; the second was to extend the absorption time and preserve total exposure as measured by the AUCs. The expectation was that lower Cₘₐₓ would improve overall tolerability and extended absorption time with preserved AUCs was expected to decrease the variability seen in the exposures of the IR formulation. Based on the available safety, tolerability, and PK results of the 4 MR formulations administered under fasted conditions in the single-dose MR part, the formulation chosen by the Sponsor for administration under fed conditions in the single-dose MR part was the 200 mg 8-hour MR formulation. This MR formulation testing was important as it was planned to use this formulation in the Phase 2 proof-of-concept studies planned to commence in the year 2020.

A final part (multiple-dose MR part) was added assessing the safety, tolerability, and PK profile of multiple doses (every 12 hours [q12h] for 5 consecutive days) of the MR formulation with a 200 mg dose strength and an 8-hour dissolution profile (200 mg 8-hour MR formulation), taken with food. This dose and formulation were tested in the fasted and fed states in the single-dose MR part. This dose and formulation are targeted for use in a patient population in later studies, and data collected from the subject cohort in this final part would inform that decision.

Study Objectives

### Primary

To assess the safety and tolerability of single oral doses of MR formulations of IMB-1018972, and multiple oral doses of the 200 mg 8-hour MR formulation of IMB-1018972 in healthy subjects.

### Secondary

To assess the PK profile of single oral doses of MR formulations of IMB-1018972, and multiple oral doses of the 200 mg 8-hour MR formulation of IMB-1018972 in healthy subjects
To assess the absorption and PK profile of the 200 mg 8-hour MR formulation of IMB-1018972 following multiple oral doses taken with food in healthy subjects
To evaluate the safety and tolerability of the 200 mg 8-hour MR formulation of IMB-1018972 following multiple oral doses taken with food in healthy subjects

### Investigational Plan

### Overall Study Design and Plan

Type of Study

This was a double-blind, randomized, study consisting of single-dose and multiple-dose MR parts to assess the safety, tolerability, and PK of single oral doses of MR formulations of IMB-1018972, and multiple oral doses of the 200 mg 8-hour MR formulation of IMB-1018972. The study started with the SAD part.

Single-Dose MR Part

In the single-dose MR part, 1 group of 12 healthy subjects (all on active drug) was included. The subjects received a single oral dose of 1 of 4 MR formulations of IMB-1018972 under fasted conditions (an overnight fast of at least 10 hours) on Days 1, 4, 7, and 10 in a fixed order that was the same for all subjects. The MR formulation of IMB-1018972 to be administered on Day 13 under fed conditions was 1 of the 4 MR formulations administered on Days 1, 4, 7, and 10 under fasted conditions as determined by the Sponsor based on the available safety, tolerability, and PK results of the 4 MR formulations.

The single-dose MR part consisted of:
An eligibility screening period of up to 35 days
One study period involving administration of single doses of IMB-1018972 on Days 1, 4, 7, 10, and 13
Safety assessments and blood sampling for PK purposes from predose on Day 1 up to 48 hours after the last drug administration
Discharge at 72 hours after the last study drug administration
A follow-up visit 7 to 14 days after the last PK blood sample

Multiple Dose MR Part

In the multiple-dose MR part, 1 group of 12 healthy subjects (all on active drug) was included. Subjects received multiple oral doses of the MR formulation of IMB-1018972 q12h under fed conditions for 5 consecutive days; on Day 5 only a single morning dose was administered. The MR formulation of IMB-1018972 administered was the same as that administered in the single-dose MR part under both fasted and fed conditions.

The multiple-dose MR part consists of:
An eligibility screening period of up to 35 days
One study period involving administration of multiple doses of the 200 mg 8-hour MR formulation of IMB-1018972 from Day 1 to Day 5
Safety assessments and blood sampling for PK purposes from predose on Day 1 up to 48 hours after the last drug administration
Discharge at 48 hours after the last drug administration
A follow-up visit 6 to 8 days after the last PK blood sample

Screen Period

Subjects reported to the medical screening facility for the eligibility screening within 5 weeks prior to (the first) study drug administration.

Subjects signed the study-specific ICF prior to any study-specific screening procedures being performed. The written informed consent was obtained for all subjects, regardless of their eligibility for the study. The signed ICFs were retained and archived at PRA and a copy was provided to the subject.

Treatment Period

Subjects were in the clinic for 1 treatment period. The subjects were admitted to the clinical research center in the afternoon of Day −1. Day 1 was the day of (the first) drug administration.

Subjects of the single-dose MR part were discharged on Day 16 (72 hours after the last study drug administration on Day 13) after completion of the assessments. Subjects of the multiple-dose MR part were discharged on Day 7 (48 hours after the last study drug administration on Day 5) after completion of the assessments

Follow-Up

For the single-dose MR part, the follow-up assessments were performed 7 to 14 days after the last PK blood sample (between Day 23 and Day 30). For the multiple-dose MR part, the follow-up assessments were performed 6 to 8 days after the last PK blood sample (Day 14±1 day).

### Discussion of Study Design

Effect of Food

In the single-dose MR part, 12 healthy subjects (all on active drug) received a single oral dose of 1 of 4 MR formulations of IMB-1018972 under fasted conditions (an overnight fast of at least 10 hours) on Days 1, 4, 7, and 10 in a fixed order that was the same for all subjects. On Day 13, 1 of these 4 MR formulations was chosen to be administered to the same subjects under fed conditions (FDA-defined high-fat breakfast prior to dosing) to evaluate the possible effect of food on the PK of IMB-1018972). This allowed for a within-subject comparison of the PK of IMB-1018972 in plasma and tolerability after administration of this MR formulation in fasted and fed conditions.

In the multiple-dose MR part, 12 healthy subjects received multiple oral doses of the MR formulation of IMB-1018972 q12h under fed conditions for 5 consecutive days; on Day 5 only a single morning dose was administered. The MR formulation of IMB-1018972 administered was the same as that administered in the single-dose MR part. The safety, tolerability, and PK of multiple doses of this MR formulation taken with food were evaluated.

Other

The planned confinement period, day of discharge, and follow-up period could be adapted depending on emerging study results. Also, the timing, type, and number of safety and PK assessments could be changed during the study.

There was no indication from in vitro studies (cytochrome P450 [CYP]3A4/GT1A1/CYP2C19/CYP2C9) for interaction with oral contraceptives. Women of childbearing potential who were using adequate contraception were included in the present study, in order to make the outcome of this FIH study relevant for the female target patient population.

The use of healthy subjects as opposed to patients allowed a clearer interpretation of the study results, as there were no confounding factors resulting from changes in disease state and/or concomitant medications.

The study was performed in different groups of subjects since the number of doses to be tested, and all assessments associated with these sessions, were regarded as too extensive to be performed in a single group of subjects participating repeatedly.

The Investigator took all the usual precautions necessary for studies at an early stage in the development of a new drug.

### Selection of Study Population

The overall study population consisted of 88 subjects.

In the single-dose and multiple-dose MR parts, a total of 24 healthy male or female subjects (12 in each part) were included. All efforts were made to have a ratio of 50:50 for male and female subjects, but at minimum at least 4 subjects of each gender were dosed in each part.

Inclusion Criteria

Subjects were eligible for inclusion in the study if they met all the following inclusion criteria:
1. Gender: male or female.
2. Age: 18 years to 65 years, inclusive, at screening.
3. Body mass index (BMI): 18.0 kg/m2 to 32.0 kg/m2, inclusive.
4. Status: healthy subjects.
5. At screening, females could be of childbearing potential (but not pregnant or lactating), or of nonchildbearing potential (either surgically sterilized or physiologically incapable of becoming pregnant, or at least 1 year postmenopausal [amenorrhea duration of 12 consecutive months]); nonpregnancy was confirmed for all females by a serum pregnancy test conducted at screening and each admission.
6. Female subjects of childbearing potential who had a fertile male sexual partner had to agree to use adequate contraception from screening until 90 days after the follow-up visit. Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
7 Male subjects, if not surgically sterilized, had to agree to use adequate contraception and not donate sperm from (first) admission to the clinical research center until 90 days after the follow-up visit. Adequate contraception for the male subject (and his female partner) was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
8. All prescribed medication had to be stopped at least 30 days prior to (first) admission to the clinical research center. An exception was made for hormonal contraceptives, which could be used throughout the study.
9. All over-the-counter medication, vitamin preparations and other food supplements, or herbal medications (e.g., St. John's Wort) had to be stopped at least 14 days prior to (first) admission to the clinical research center. An exception was made for paracetamol, which was allowed up to admission to the clinical research center.
10. Willingness to abstain from alcohol, methylxanthine-containing beverages or food (coffee, tea, cola, chocolate, energy drinks), grapefruit (juice), and tobacco products from 48 hours prior to (each) admission to the clinical research center.
11. Good physical and mental health on the basis of medical history, physical examination, clinical laboratory, and vital signs, as judged by the Investigator.
12. Had no clinically significant abnormal 12-lead ECG (incomplete right bundle branch block could be accepted) at screening: PR-interval <210 ms, QRS-duration <120 ms, and QTc-interval (Fridericia's)≤450 msec for males and females.
13. Willing and able to sign the ICF.

Exclusion Criteria

Subjects were excluded from participation if any of the following exclusion criteria applied:
1. Previous participation in the current study.
2. Employee of PRA or the Sponsor.
3. History of relevant drug and/or food allergies.
4. Using tobacco products within 3 months prior to (the first) drug administration.
5. History of alcohol abuse or drug addiction (including soft drugs like cannabis products).
6. Positive drug and alcohol screen (opiates, methadone, cocaine, amphetamines [including ecstasy], cannabinoids, barbiturates, benzodiazepines, tricyclic antidepressants, and alcohol) at screening and (each) admission to the clinical research center.
7. Average intake of more than 24 units of alcohol per week (1 unit of alcohol equals approximately 250 mL of beer, 100 mL of wine, or 35 mL of spirits).
8. Positive screen for hepatitis B surface antigen (HBsAg), anti-hepatitis C virus (HCV) antibodies, or anti-HIV 1 and 2 antibodies.
9. Participation in a drug study within 60 days prior to (the first) drug administration in the current study. Participation in more than 4 other drug studies in the 12 months prior to (the first) drug administration in the current study.
10. Donation or loss of more than 100 mL of blood within 60 days prior to (the first) drug administration. Donation or loss of more than 1.5 liters of blood (for male subjects)/more than 1.0 liters of blood (for female subjects) in the 10 months prior to (the first) drug administration in the current study.
11. Significant and/or acute illness within 5 days prior to (the first) drug administration that could impact safety assessments, in the opinion of the Investigator.
12. Unsuitable veins for infusion or blood sampling.

Please note that subjects were to refrain from consumption of any foods containing poppy seeds within 48 hours (2 days) prior to screening to the clinical research center to avoid false positive drug screen results. In addition, they were to refrain from strenuous exercise within 96 hours (4 days) prior to screening as this could result in abnormal clinical laboratory values.

Removal of Subject from Assessment

Participation in the study was strictly voluntary. A subject had the right to withdraw from the study at any time for any reason.

The Investigator had the right to terminate participation of a subject for any of the following reasons: difficulties in obtaining blood samples, violation of the protocol, severe AEs or SAEs, or for any other reason relating to the subject's safety or the integrity of the study data.

If a subject was withdrawn from the study, the Sponsor was to be informed immediately. If there was a medical reason for withdrawal, the subject remained under the supervision of the Investigator until satisfactory health had returned.

Subjects who dropped out or withdrew for any reason without completing all screening evaluations successfully, were considered screening failures.

A subject who was withdrawn or voluntarily withdrew from the study for any reason, whether related to the study drug or not, after having received a subject number, was considered an early-termination subject. If a subject was withdrawn for a reason related to the study drug, according to the judgment of the Investigator, the early-termination subject was not replaced. If a subject did not complete the study for a reason not related to the study drug, the early-termination subject could be replaced after mutual agreement between the Sponsor and PRA.

The decision regarding the replacement of subjects was documented.

PRA made every effort to ensure that early-termination subjects who had received study drug completed the safety follow-up assessments.

Stopping Rules for Individual Subjects

Dosing of a subject was stopped at any time during the study if any of the following circumstances occurred:
A serious adverse reaction (i.e., an SAE considered at least possibly related to the study drug administration).
An overall pattern of clinically significant changes in any safety parameter (e.g., moderate or severe AEs in >1 subject) that could appear to be minor in terms of an individual event but, in the opinion of the Sponsor or Investigator, collectively represented a safety concern.
Other findings that, at the discretion of the Investigator and/or Sponsor's Medical Monitor, indicated that further dosing should be stopped.

### Treatments

Single-Dose MR Part

The formulation chosen for administration on Day 13 was the 200 mg 8-hour MR formulation as determined by the Sponsor based on the available safety, tolerability, and PK results of the 4 MR formulations administered on Days 1, 4, 7, and 10 under fasted conditions.

The following treatments were administered in the single-dose MR part:

Day 1: single oral dose of 50 mg 8-hour MR formulation of IMB-1018972 (n=12) under fasted conditions
Day 4: single oral dose of 50 mg 4-hour MR formulation of IMB-1018972 (n=12) under fasted conditions
Day 7: single oral dose of 200 mg 8-hour MR formulation of IMB-1018972 (n=12) under fasted conditions
Day 10: single oral dose of 200 mg 4-hour MR formulation of IMB-1018972 (n=12) under fasted conditions
Day 13: single oral dose of 200 mg 8-hour MR formulation of IMB-1018972 (n=12) under fed conditions

Multiple-Dose MR Part

Twelve subjects received multiple oral doses of the 200 mg 8-hour MR formulation of IMB-1018972 q12h under fed conditions for 5 consecutive days; on Day 5 only a single morning dose was administered.

### Identity of Investigational Products

Active Medication
Drug product: IMB-1018972
Activity: Fatty acid oxidation inhibitor
In development for: Ischemic cardiovascular disease
Strength: 50 mg MR formulation and 200 mg MR formulation with 4-hour and 8-hour dissolution profile (based on free base)
Dosage form: oral MR tablet(s) to be used in the MR parts
Manufacturer: Pharmacy at PRA
Batch number: 2479-1810-00441 (drug substance)

Active Medication
Drug product: Vastarel MR (trimetazidine dihydrochloride)
Activity: Fatty acid oxidation inhibitor
In development for: Angina pectoris
Strength: 35 mg
Dosage form: Oral MR tablet
Manufacturer: Servier Research & Pharmaceuticals (Pakistan) (Pvt.) Ltd.
Batch number: 273782 (drug product)

The study drug was stored in the pharmacy at PRA in a locked facility under the required storage conditions with continuous monitoring. The study drug was dispensed by the pharmacist to the Investigator or authorized designee.

Method of Assignment Subjects to Treatment Groups

After obtaining informed consent, subjects were screened according to the inclusion and exclusion criteria. Subjects who met all eligibility criteria received a subject number upon inclusion in the study. They received the subject number just prior to dosing according to the randomization code generated by the Biostatistics Department of PRA. The subject number ensured identification throughout the study.

Subject numbers 501 to 512 for the single-dose MR part, and 513 to 524 for the multiple-dose MR part.

Any replacement subject was to receive the number of the subject to be replaced, increased by 200, and was to be administered the same treatment(s). Subjects were assigned to a study part and group based on their availability. Treatments within a group were assigned according to the randomization code generated by the Biostatistics Department of PRA.

In both MR parts, all 12 subjects received IMB-1018972.

Subjects who dropped out or withdrew for any reason without completing all screening evaluations successfully were considered screening failures. Such subjects, and also subjects who were eligible for inclusion in the study but did not receive the study drug, received no subject number, and only applicable data were entered in the eCRFs.

Selection of Doses in the Study

Based on the nonclinical toxicology data, it was considered that subjects in this clinical study were not at unreasonable risk of adverse effects. Based on the 28-day dog no observed adverse effect level (NOAEL) of 200 mg/kg/day (oral), the calculated human equivalent dose (HED) is 108 mg/kg/day. For a 60-kg individual, the NOAEL dose would be 6480 mg. With a 10-fold safety factor applied, this would allow for a maximum recommended starting dose (MRSD) of 648 mg/day. 7,8 The planned starting dose in the current Phase 1 study was 50 mg, equivalent to 0.83 mg/kg/day for a 60-kg subject. This starting dose is less than 10% of the MRSD determined from the dog NOAEL and less than 1% of the dog NOAEL

The maximum planned dose in this study of 1600 mg in healthy volunteers was 25% of the HED NOAEL dose of 6480 mg and only 2.5 fold higher than the MRSD. The conservative dosing margin was expected to cover potential supratherapeutic exposures, for instance in patients with renal or hepatic impairment, or in case of potential drug interactions with IMB-1018972. This risk for healthy volunteers at these exposure levels was determined to be acceptable based on the absence of irreversible or significant toxicities without sentinel safety biomarkers.

The relevant animal study was the 28-day dog study where the NOAEL for IMB-1018972 was 200 mg/kg/day. The AUC_{0-8×2} for IMB-1028814 on Day 26 at this dose was 417,733 and 652,849 ng*h/mL for males and females, respectively. The AUC_{0-8×2} for trimetazidine on Day 26 at this dose was 15,042, and 13,834 ng*h/mL for males and females, respectively.

A cohort was added by the Sponsor that was testing a single 35 mg MR dose of trimetazidine (Vastarel). This dose was selected as it is the most commonly used dose of trimetazidine in treating angina and it was therefore known that it has an efficacious PK profile.

Timing of Doses in the Study

The study drug was administered with 240 mL of tap water to the subject in the upright position. If needed, an additional volume of water was allowed to consume the capsules/tablets comfortably; this additional volume was documented in the eCRF. The dose was given between 08:00 h and 11:00 h, and between 20:00 h and 23:00 h for the afternoon/evening dose. Dosing for each individual subject was at around the same time (±15 min) on each dosing day. The study drug was not chewed.

Administration of the study drug was supervised by the Investigator or authorized designee. After drug administration, a mouth and hand inspection took place.

### Dosing Under Fasted Conditions

Single-Dose MR Part

Before dosing (on Days 1, 4, 7, and 10 in the single-dose MR part), subjects fasted overnight for at least 10 hours following a light supper on the evening before. Following dosing, subjects fasted for 4 hours until lunch. During fasting, fluids other than water were not allowed; however, water was not allowed from 2 hours predose until 1 hour postdose (apart from the water taken with the dose).

Subjects of the single-dose MR part were not allowed to lie down for 4 hours after dosing, except when required for assessments that needed to be performed.

### Dosing Under Fed Conditions

Single-Dose MR Part Before dosing on Day 13, subjects fasted overnight for at least 10 hours following a snack on the evening before. On Day 13, subjects received a FDA-defined high-fat breakfast that had to be consumed within 20 minutes. Dosing occurred at 30 minutes after the start of breakfast. Following dosing, subjects fasted for 4 hours until lunch. During fasting, fluids other than water were not allowed. Subjects were not allowed to lie down for 4 hours after dosing, except when required for assessments that needed to be performed.

Multiple-Dose MR Part

### Morning Dose

Before each morning dose, subjects fasted overnight for at least 10 hours following a snack the evening before. On Days 1 and 5, subjects received a standardized breakfast that had to be consumed within 20 minutes. Dosing took place 30 minutes after the start of breakfast. After dosing, subjects fasted for 4 hours until lunch. During fasting, fluids other than water were not allowed. On Days 2 to 4, breakfast was not standardized, and was given maximally within 1 hour before dosing and consumed before dosing. Subjects did not fast after dosing on these days.

### Evening Dose

From Day 1 to Day 4, an evening snack was given maximally within 1 hour before dosing and consumed before dosing. Subjects fasted overnight for at least 10 hours after consuming the snack.

Subjects were not allowed to lie down for 4 hours after morning or evening dosing, except when required for assessments that needed to be performed.

Meals During the Study

A fasting period of at least 4 hours was required before obtaining clinical laboratory samples at all time points.

When not fasting, meals and snacks (such as decaffeinated coffee, herbal tea, fruit, and biscuits) were provided according to PRA standard operating procedures (SOPs). A light supper was provided on the evening before those days where fasting was required until lunch time (fasted conditions); a snack was provided on the evening before those days where fasting was required until the FDA-defined high-fat breakfast or breakfast (fed conditions).

For Day 13 of the single-dose MR part, the FDA-defined high-fat breakfast of 918 kcal consisted of:
2 fried eggs (in 15 g butter/margarine) (approximately 100 g)
1 portion of bacon (40 g) (or brie 60+ for vegetarians)
1 portion of fried potatoes (115 g)
2 slices of (toasted) (wheat) bread (approximately 70 g) with 15 g margarine
1 glass of whole milk (240 mL)
   The total of 918 kcal (vegetarian version 915 kcal) could be broken down as follows:
39 g protein=156 kcal
59 g fat=527 kcal
59 g carbohydrates=235 kcal

Blinding

In both MR parts, all 12 subjects received IMB-1018972.

The Bioanalytical Laboratory of PRA where the PK samples were analyzed was provided a copy of the randomization code by the pharmacy since only samples of subjects who had received the active drug IMB-1018972 were to be analyzed.

Previous and Concomitant Therapy and Other Restrictions During the Study

The use of all prescribed medication was not allowed from (first) admission to the clinical research center until follow-up. An exception was made for hormonal contraceptives, which were allowed throughout the study. The use of all over-the-counter medication, vitamin preparations and other food supplements, or herbal medications (e.g., St. John's Wort) was not allowed from (first) admission to the clinical research center until follow-up. An exception was made for paracetamol: from (first) admission onwards, the Investigator could permit a limited amount of paracetamol for the treatment of headache or any other pain. Other medication to treat AEs could only be prescribed if deemed necessary by the Investigator. If medication was used, the name of the drug, the dose, and dosage regimen were recorded in the eCRF.

The use of alcohol, methylxanthine-containing beverages or food (coffee, tea, cola, chocolate, energy drinks), grapefruit (juice), and tobacco products was not allowed during the stay in the clinical research center.

Strenuous exercise was not allowed within 96 hours (4 days) prior to (each) admission and during the stay(s) in the clinical research center.

Subjects were not allowed to consume any foods containing poppy seeds within 48 hours (2 days) prior to (each) admission to the clinical research center as this could cause a false positive drug screen result.

Female subjects of childbearing potential, with a fertile male sexual partner, were required to use adequate contraception (see description below) from screening until 90 days after the follow-up visit.

Male subjects, if not surgically sterilized, were required to use adequate contraception (see description below) and not donate sperm from (first) admission to the clinical research center until 90 days after the follow-up visit.

Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.

Subjects were not allowed to donate blood during the study until the follow-up visit (other than the blood sampling planned for this study).

Treatment Compliance

Study drug was administered in the clinical research center. To ensure treatment compliance, administration of the study drug was supervised by the Investigator or authorized designee. Compliance was further confirmed by bioanalytical assessment of IMB-1018972, IMB-1028814, and trimetazidine in plasma and urine samples.

The exact times of study drug administration and the number of units administered were recorded in the eCRF. Drug accountability procedures as specified in the CSP were followed.

### Safety and Pharmacokinetic Measurements and Variables

The present study was performed to assess safety, tolerability, and PK following single oral doses of MR formulations of IMB-1018972, and multiple oral doses of the 200 mg 8-hour MR formulation of IMB-1018972. This study did not comprise efficacy or pharmacodynamic assessments.

Adverse Events

AEs were recorded from (first) admission until completion of the follow-up visit. Any clinically significant observations in results of clinical laboratory, 12-lead ECGs, vital signs, or physical examinations were recorded as AEs.

A treatment-emergent AE (TEAE) was defined as any event not present prior to (the first) administration of the study drug or any event already present that worsened in either severity or frequency following exposure to the study drug.

An AE that occurred prior to (the first) administration of the study drug was considered a pretreatment AE.

At several time points before and after drug administration, subjects were asked nonleading questions to determine the occurrence of AEs. Subjects were asked in general terms about any AEs at regular intervals during the study. In addition, all AEs reported spontaneously during the course of the study were recorded. Details included description of the event, date and time of onset, date and time of end, total duration, severity, relationship to study drug, intervention, seriousness, and outcome. All answers were interpreted by the Investigator and were recorded in the eCRF. All AEs were classified according to the Medical Dictionary for Regulatory Activities (MedDRA; Version 22.0) for AEs.

The severity of the AEs was rated as mild, moderate, or severe; the relationship between the AEs and the study drug was indicated as none, unlikely, possibly, likely, or definitely. Adverse events assessed as possibly, likely, or definitely were considered related to the study drug; AEs assessed as none or unlikely were considered not related to the study drug.

Concomitant medication or other therapy required in case of any AEs was recorded. Concomitant medications were classified according to the World Health Organization Drug Dictionary (Version 22.0).

All AEs were followed up until their resolution or stabilization.

Clinical Laboratory

Blood and urine samples for clinical laboratory assessments were collected according to PRA SOPs.

The following parameters were measured:
Clinical chemistry (serum quantitatively): total bilirubin, alkaline phosphatase, gamma glutamyl transferase, aspartate aminotransferase (AST), alanine aminotransferase (ALT), lactate dehydrogenase, creatinine, urea, total protein, glucose, inorganic phosphate, sodium, potassium,
calcium, and chloride
Hematology (blood quantitatively): leukocytes, erythrocytes, hemoglobin, hematocrit, thrombocytes, partial automated differentiation (lymphocytes, monocytes, eosinophils, basophils, and neutrophils), mean corpuscular volume, mean corpuscular hemoglobin, and mean corpuscular hemoglobin concentration
Coagulation (blood quantitatively): prothrombin time (reported in seconds and as international normalized ratio), activated partial thromboplastin time, and fibrinogen
Urinalysis (urine qualitatively): hemoglobin, urobilinogen, ketones, glucose, and protein
Serology: HBsAg, anti-HCV, and anti-HIV 1 and 2
Drug and alcohol screen: opiates, methadone, cocaine, amphetamines (including ecstasy), cannabinoids, barbiturates, benzodiazepines, tricyclic antidepressants, and alcohol
Pregnancy test (females only): (3-human chorionic gonadotropin in serum

Urine for urinalysis was taken from the PK urine collection container at the end of a collection interval.

In case of unexplained or unexpected clinical laboratory test values, the tests were repeated as soon as possible and followed up until the results had returned to the normal range and/or an adequate explanation for the abnormality was found. The clinical laboratory clearly marked all laboratory test values that were outside the normal range, and the Investigator indicated which of these deviations were clinically significant. Clinically significant laboratory result deviations were recorded as AEs and the relationship to the treatment was indicated.

Vital Signs

Systolic and diastolic blood pressure and pulse were recorded after the subject had been resting for at least 5 minutes in the supine position. These assessments were made using an automated device. Body temperature and respiratory rate were measured subsequently.

Electrocardiogram

A standard 12-lead ECG was recorded after the subject had been resting for at least 5 minutes in the supine position. The ECG was recorded using an ECG machine equipped with computer-based interval measurements (with no/minimal disturbance by procedures). The following ECG parameters were recorded: heart rate, PR-interval, QRS-duration, QT-interval, QTcF-interval, and the interpretation of the ECG profile by the Investigator.

Continuous Cardiac Monitoring (Telemetry)

In the single-dose and multiple-dose MR parts, no telemetry was performed.

All relevant or significant arrhythmic events were recorded in rhythm strips (10 seconds). The ECG was evaluated by the Investigator for clinically significant events.

Physical Examination

Physical examination was performed according to PRA SOPs. In addition, body weight and height were measured according to PRA SOPs.

Pharmacokinetic Measurements

Blood Sampling

At the time points defined in the schedules of assessments, blood samples of 3 mL per time point were taken for the analysis of IMB-1018972, IMB-1028814, and trimetazidine in plasma samples. The blood samples were taken via an indwelling intravenous catheter or by direct venipuncture. The exact times of blood sampling were recorded in the eCRF.

During days with telemetry, subjects remained quietly supine (with no/minimal disturbance by procedures) for 10 minutes followed by an up to 5-minute period for each ECG assessment that was planned just prior to PK sampling. Start and stop time of the (in total) 15-minute periods were recorded.

Details on sample collection, sample aliquoting, sample handling, sample storage, and sample shipping can be found in the laboratory manual prepared by PRA.

Plasma samples may (in the future) also be used for research purposes such as evaluation of the activity of IMB-1018972 and trimetazidine, identification of exploratory biomarkers that are predictive of activity, cytochrome P450 profiling, or other exploratory evaluations that may help characterize the molecular mechanisms of IMB-1018972 and trimetazidine. The samples will be stored for a maximum of 15 years for this purpose.

Urine Collection

During the intervals defined in the schedules of assessments, urine was collected for the analysis of IMB-1018972, IMB-1028814, and trimetazidine. The subjects were instructed to empty their bladders completely before study drug administration and at the end of each collection interval. A blank urine sample was collected within 12 hours prior to study drug administration. The exact times of urine collection and the urine weight of the entire interval (before and after addition of any urine stabilizers, if used) were recorded in the eCRF.

Details on sample collection, sample aliquoting, sample handling, sample storage, and sample shipping can be found in the laboratory manual prepared by PRA.

Urine samples could be kept for a maximum of 1 year for further analysis of metabolites in urine in case unknown metabolites were found in plasma.

Genotyping

At the time points defined in the schedules of assessments, a blood sample of a maximum of 7 mL was collected for genotyping to better understand the effects of genotype, such as CYP alleles, on PK data. This blood sample was optional for subjects that had already been screened prior to IEC approval of protocol Version 3.0 (25 Mar. 2019), whereas it was mandatory for subjects participating in this study that had been screened after IEC approval of protocol Version 3.0 (25 Mar. 2019).

The blood sample was double coded (1 code at PRA and 1 code at the Sponsor), and the sample was kept until 15 years after completion of the study.

The blood sample was taken via an indwelling intravenous catheter or by direct venipuncture. The exact time of blood sampling was recorded in the eCRF.

Details on sample collection, sample aliquoting, sample handling, sample storage, and sample shipping can be found in the laboratory manual prepared by PRA.

Safety and Pharmacokinetic Variables

The safety variables to be measured included:
AEs
Clinical laboratory
Vital signs
12-lead ECG
Continuous cardiac monitoring (telemetry)
Physical examination

Pharmacokinetic Variables

Pharmacokinetic variables were the plasma and urine concentrations of IMB-1018972, IMB-1028814, and trimetazidine, and their PK parameters. The PK parameters that were determined or calculated using noncompartmental analysis are given in Table

TABLE 11
Plasma IMB-1018972, IMB-1028814, and Trimetazidine Parameters

| | | MD- | MD- | |
|---|---|---|---|---|
| | SD- | MR | MR | |
| Parameter | MR | Day1 | Day 5 | Description |
| Cₘₐₓ | X | X | X | Maximum plasma concentration. |
| | | | | Observed peak analyte concentration |
| | | | | obtained directly from the experimental |
| | | | | data without interpolation, expressed in |
| | | | | concentration units. |
| Cₘᵢₙ | | | X | Minimum plasma concentration (predose |
| | | | | concentration excluded). |
| tₘₐₓ | X | X | X | Time to maximum plasma concentration. |
| | | | | First observed time to reach peak analyte |
| | | | | concentration obtained directly from the |
| | | | | experimental data without interpolation, |
| | | | | expressed in time units. |
| AUC₀₋ₜ | X | | | Area under the plasma concentration- |
| | | | | time curve (time 0 to time of last |
| | | | | quantifiable concentration). |
| AUC_{0-inf} | X | | | Area under the plasma concentration- |
| | | | | time curve (time 0 to infinity). Percent |
| | | | | extrapolation less than or equal to 20% |
| | | | | is required to obtain a reliable AUC_{0-inf} |
| % AUCₑₓₜᵣₐ | X | | | Percentage of estimated part of the |
| | | | | calculation of AUC_{0-inf}. Calculated as: |
| | | | | ([AUC_{0-inf} − AUC₀₋ₜ]/AUC_{0-inf}) * |
| | | | | 100%. |
| AUC_{0-T} | | X | X | Area under the plasma concentration- |
| | | | | time curve over the dosing interval of |
| | | | | 0-12 hours postmorning dose. |
| kₑₗ | X | | X | Terminal elimination rate constant |
| | | | | calculated by linear regression of the |
| | | | | terminal log-linear portion of the |
| | | | | concentration vs time curve. Linear |
| | | | | regression of at least 3 points and an |
| | | | | adjusted r² greater than 0.80 were |
| | | | | required to obtain a reliable kₑₗ. |
| t_{1/2} | X | | X | Terminal elimination half-life |
| | | | | expressed in time units. |
| | | | | Percent extrapolation less than or |
| | | | | equal to 20% and adjusted r² |
| | | | | greater than 0.80 was required to |
| | | | | obtain a reliable t_{1/2}. |
| CL/F | X | | | Apparent oral clearance, |
| | | | | calculated as dose/AUC_{0-inf} IMB- |
| | | | | 1028814 only, assuming 100% IMB- |
| | | | | 1018972 was converted to IMB- |
| | | | | 1028814. |
| CLₛₛ/F | | | X | Apparent oral clearance at steady |
| | | | | state, calculated as dose/AUC_{0-T}. The |
| | | | | AUC_{0-T} after the morning dose was used |
| | | | | in the calculation. IMB-1028814 only, |
| | | | | assuming 100% IMB-1018972 was |
| | | | | converted to IMB-1028814. |
| V_{z}/F | X | | X | Apparent volume of distribution at |
| | | | | terminal phase, calculated as (CL/F)/kₑₗ |
| | | | | (SAD/FE/MR), or as (CLss/F)/kₑₗ |
| | | | | (MAD). For IMB-1028814 only. |
| R_{ac} | | | X | Accumulation ratio, based on AUC_{0-T}, |
| | | | | of Day 14 vs Day 1 (AUC_{0-T} after |
| | | | | morning dose). |

FE = food effect;
MAD = multiple ascending dose;
SAD = single ascending dose;
MD = multiple dose;
MR = modified release;
SD = single dose

The sum of IMB-1028814 and trimetazidine concentrations and PK parameters was calculated corrected for molecular weights of 310 kDa for IMB-1028814 and 266 kDa for trimetazidine.

Plasma trough levels of IMB-1018972, IMB-1028814, and trimetazidine were also determined (MAD part only).

The AUCs were calculated using the linear up/log down trapezoidal rule, expressed in units of concentration x time.

TABLE 12
Urine IMB-1018972, IMB-1028814, and Trimetazidine Parameters

| | SAD/FE | |
|---|---|---|
| Parameter | (first period) | Description |
| Aeᵤᵣᵢₙₑ | X | Total amount of drug excreted unchanged into urine to time t |
| | | (time of last measurable concentration), obtained by adding the |
| | | amounts excreted over each collection interval. |
| Feᵤᵣᵢₙₑ | X | Fraction (%) of the administered dose excreted unchanged into |
| | | urine. Calculated as: Feᵤᵣᵢₙₑ = (Aeᵤᵣᵢₙₑ/Dose) * 100. |
| CL_{R} | X | Renal clearance. Calculated as Aeᵤᵣᵢₙₑ/AUC₀₋ₜ. |

Drug Concentration Measurements

The analysis of IMB1018972, IMB-1028814, and trimetazidine in plasma and urine samples was performed at the Bioanalytical Laboratory of PRA using validated liquid chromatography-mass spectrometry/mass spectrometry methods. The results from calibration samples and quality control samples demonstrated acceptable performance of the methods throughout the experimental period. Data on the performance of the method and stability indicate that the sample results as reported are reliable.

### Statistical and Analytical Plan for Safety and Pharmacokinetic Evolution

Safety Set

All subjects who had received at least 1 dose of IMB101897 or trimetazidine.

Pharmacokinetic Set

All subjects who had received at least 1 dose of IMB-1018972 or trimetazidine and provided sufficient bioanalytical assessment results to calculate reliable estimates of the PK parameters.

Statistical and Analytical Plan for Safety and Pharmacokinetic Evaluation

Details on the preparation of the listings and summary tables and figures can be found in the SAP and was generated by the Biostatistics Department of PRA. The SAP was finalized prior to database lock (and unblinding of study treatment codes).

All safety and PK data were listed. In addition, all data were summarized in tabular and/or graphical form and descriptive statistics were given, as appropriate.

Evolution of Safety and Tolerability

Safety and tolerability were assessed through AEs, clinical laboratory, vital signs, ECGs, continuous cardiac monitoring (telemetry), and physical examination findings, and any other parameter that was relevant for safety assessment.

All individual safety results were listed and descriptive statistics including change from baseline were calculated, where applicable.

Pharmacokinetic Evaluation

Descriptive statistics (number, arithmetic mean, SD, coefficient of variation, minimum, maximum, median, and geometric mean) were calculated for plasma and urine PK parameters of IMB-1028814, trimetazidine, and IMB-1028814+trimetazidine in the PK population, where applicable.

The effect of food on the relative oral bioavailability of IMB-1018972 following a single oral administration was explored. This occurred in in the single-dose MR part where subjects received the same dose, first under fasted conditions and then under fed conditions. The evaluation was based on 90% CIs for the ratio of the geometric means, based on log-transformed data, for Cₘₐₓ, AUC₀₋ₜ, and AUC_{0-inf}.

Determination of Sample Size

For this FIH study, no prospective calculations of statistical power were made. The sample size was selected to provide information on safety, tolerability, and PK following single doses of MR formulations of IMB-1018972, and multiple doses of the 200 mg 8-hour MR formulation of IMB-1018972, and is typical for a FIH study. Any p-values to be calculated according to the SAP were interpreted in the perspective of the explorative character of this study.

### Study Subjects

Subject 505 of the single-dose MR part was withdrawn from the study due to a moderate TEAE of ALT increased (possibly related; up to 149 IU/L on Day 11) and did not receive the last single oral dose of 200 mg 8-hour MR formulation of IMB-1018972 under fed conditions on Day 13. None of these discontinued subjects were replaced. All 88 subjects were included in the PK and safety sets.

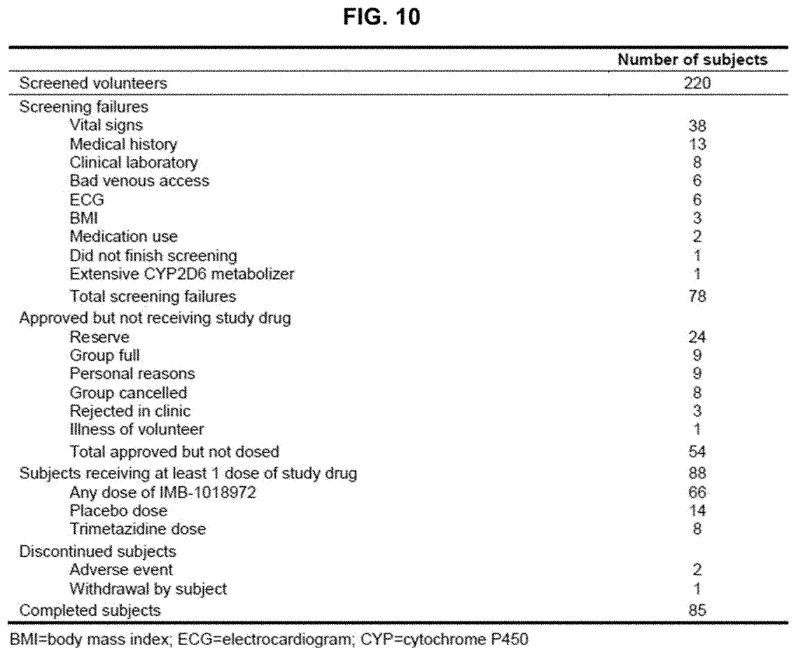

FIG. 10 is a table of the disposition of subjects.

Genotyping

All subjects provided a blood sample for genotyping. The blood sample was used to genotype subjects with a particular interest on CYP2D6 to better understand differences in the PK data. Any results of the analysis of the relationship between genotype and PK data will presented separately from this CSR.

Measurements of Treatment Compliance

Study drug was administered in the clinical research center. To ensure treatment compliance, administration of the study drug was supervised by the Investigator or authorized designee. There was no indication of noncompliance based on observations during study drug administration. In addition, bioanalytical assessment of IMB-1018972, IMB-1028814, and trimetazidine in plasma and urine samples confirmed treatment compliance.

### Clinical Laboratory Evaluation

Laboratory Values Over Time

Although several individual changes from baseline were observed in the clinical laboratory values, no clinically important trends were seen.

Individual Subject Changes

The majority of the subjects had one or more out of range values for clinical laboratory tests at various times during the study. Most of these were minor and considered by the Investigator to have no clinical implication. A number of ALT levels measured for 1 subject were above the normal range and considered to be clinically significant abnormal.

### Vital Signs, ECGs, Physical Findings, and Other Observations Related to Safety

Vital Signs

Although several individual changes from baseline were observed, blood pressure, pulse, body temperature, and respiratory rate showed no trends or clinically relevant changes during any of the study parts.

Electrocardiogram

No changes or trends of clinical significance were seen for the heart rate, PR-interval, QRS-duration, QT-interval, or QTcF-interval during any of the study parts. All 12-lead ECG evaluations were recorded as normal or, in case of abnormal recordings, these were not considered to be clinically significant.

Continuous Cardiac Monitoring (Telemetry)

All telemetric ECG evaluations obtained in the SAD and MAD parts were recorded as normal or, in case of abnormal recordings, these were not considered to be clinically significant.

Physical Examination

All abnormalities observed at screening and all changes observed after screening for physical examinations were considered to be of no clinical significance.

Tables for Modified Release Formulations

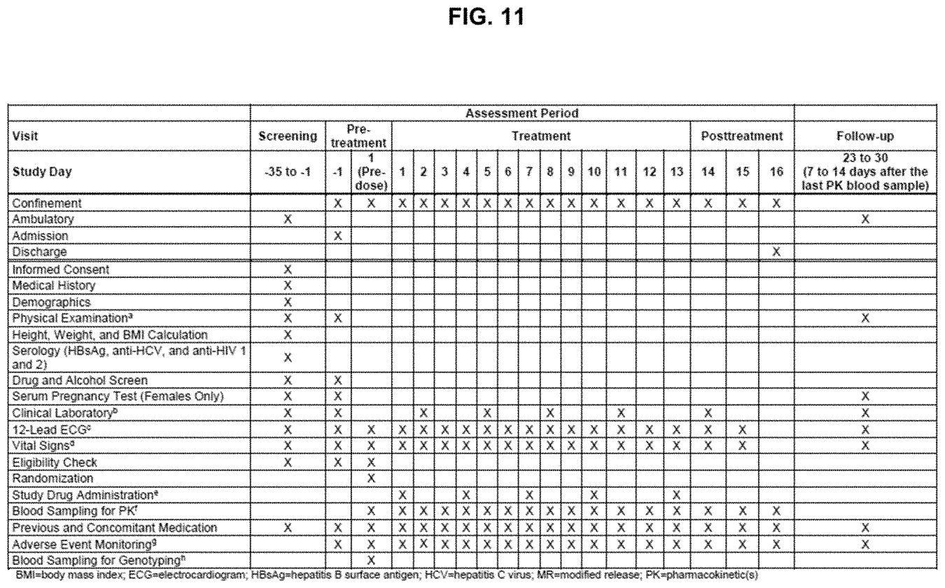

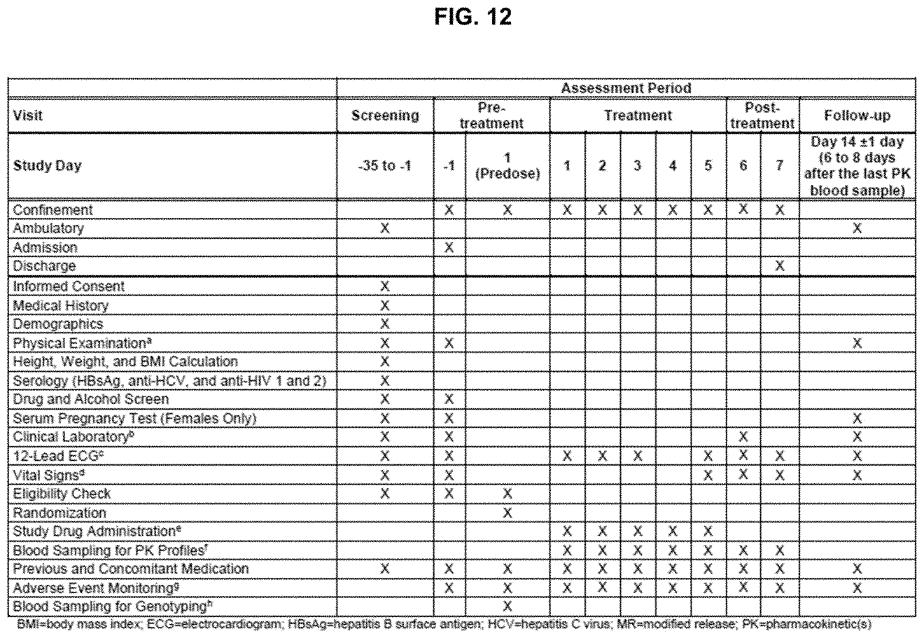

FIG. 11 is a table of assessments given for the Single-Dose MR Part, with the following notations:
a. Physical examination: at screening, on Day −1 (admission; this was a directed examination only done at the discretion of the Investigator), and at follow-up. On other days, a physical examination could be done on indication only at the discretion of the Investigator.
b. Clinical laboratory tests (including clinical chemistry, hematology, coagulation, and urinalysis): at screening, on Day −1 (admission), at 24 hours after each dose, and at follow-up.
c. 12-lead ECG: at screening, on Day −1 (admission), prior to each dose and just prior to the PK sampling time points of 1, 4, 6, 12, 24, and 48 hours after each dose, and at follow-up.
d. Vital signs (supine systolic and diastolic blood pressure, pulse, body temperature, and respiratory rate): at screening, on Day −1 (admission), prior to each dose and at 1, 4, 6, 12, 24, and 48 hours after each dose, and at follow-up.
e. The subjects received a single oral dose of 1 of 4 MR formulations of IMB-1018972 under fasted conditions (an overnight fast of at least 10 hours) on Days 1, 4, 7, and 10 in a fixed order which was the same for all subjects. The MR formulation of IMB-1018972 to be administered on Day 13 under fed conditions was 1 of the 4 MR formulations administered on Days 1, 4, 7, and 10 under fasted conditions.
f. Blood sampling for PK of IMB-1018972, IMB-1028814, and trimetazidine in plasma: prior to each dose and 0.25, 0.5, 1, 2, 3, 4, 5, 6, 8, 10, 12, 16, 24, 36, and 48 hours after each dose (and 72 hours after dosing on Day 13).
g. AEs were recorded from admission until completion of the follow-up visit.
h. Blood sampling for genotyping was mandatory.
   FIG. 12 is a table of assessments given for the Multiple-Dose MR part, with the following notations:
a. Physical examination: at screening, on Day −1 (admission; this was a directed examination only done at the discretion of the Investigator), and at follow-up. On other days, a physical examination could be done on indication only at the discretion of the Investigator.
b. Clinical laboratory tests (including clinical chemistry, hematology, coagulation, and urinalysis): at screening, on Day −1 (admission), at 24 hours after the last dose, and at follow-up.
c. 12-lead ECG: at screening, on Day −1 (admission), just before the time points of 1, 4, 6, 12, 24, and 48 hours after the first dose on Day 1 and after the last dose on Day 5, and at follow-up.
d. Vital signs (supine systolic and diastolic blood pressure, pulse, body temperature, and respiratory rate): at screening, on Day −1 (admission), before the last dose, at 1, 4, 6, 12, 24, and 48 hours after the last dose, and at follow-up.
e. Study drug (200 mg 8-hour MR formulation) was administered twice daily for 5 days; on Day 5 only a single morning dose was administered. Study drug administration was conducted under fed conditions.
f. On Day 1, blood sampling for PK of IMB-1028814 and trimetazidine was predose and 0.5, 1, 2, 4, 5, 6, 8, 10, and 12 hours postdose. The 12-hour sample was collected prior to the evening dose. On Day 5, blood sampling for PK of IMB-1028814 and trimetazidine was predose and 0.5, 1, 2, 4, 5, 6, 8, 10, 12, 16, 24, 36, and 48 hours postdose. Morning predose samples were also collected on Days 2, 3, and 4.
g. AEs were recorded from admission until completion of the follow-up visit.
h. Blood sampling for genotyping was mandatory.

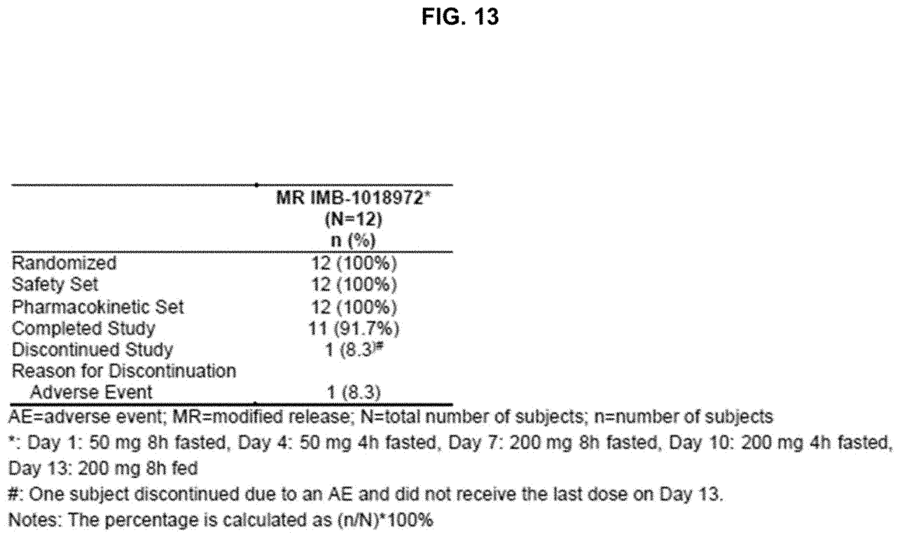

FIG. 13 is a table of analysis data sets for the Single-Dose MR Part

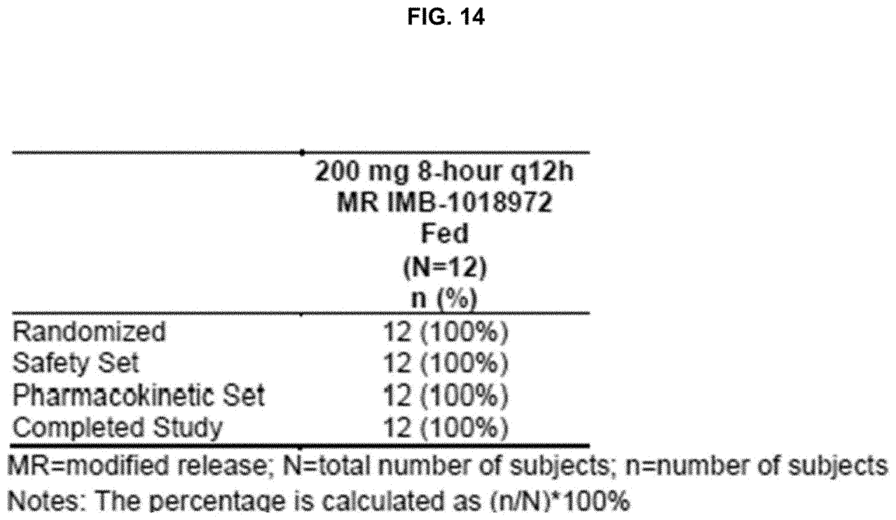

FIG. 14 a table of analysis data sets for the Multiple-Dose MR Part

### Demographic and Other Baseline Characteristics

Single-Dose MR Part

Twelve subjects were included of whom 6 were female and 6 were male. Mean age was 32 years and mean BMI was 25.8 kg/m2. Individual age ranged between 19 and 62 years and individual BMI ranged between 21.5 and 31.0 kg/m2. Eleven subjects were of white race and 1 subject was Black or African American. Eleven subjects were not of Hispanic or Latino ethnicity whereas 1 subject was of Hispanic or Latino ethnicity. The summary of the PK set was identical to that of the safety set.

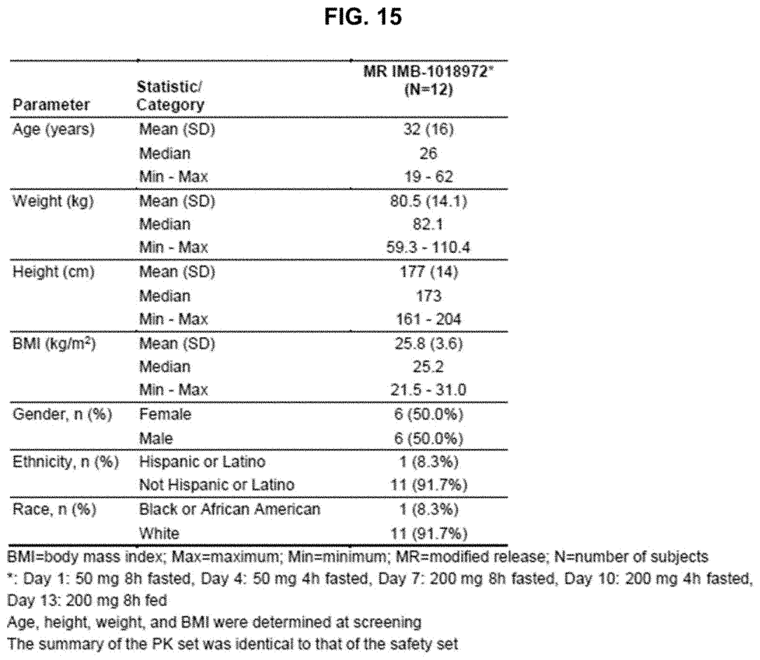

FIG. 15 is a table of a summary of demographic characteristics—Single-Dose MR Part (Safety Set).

Multiple-Dose MR Part

Twelve subjects were included of whom 6 were female and 6 were male. Mean age was 45 years and mean BMI was 25.1 kg/m2. Individual age ranged between 24 and 64 years and individual BMI ranged between 20.0 and 29.2 kg/m2. Eleven subjects were of white race and 1 subject was Asian. None of the 12 subjects were of Hispanic or Latino ethnicity. The summary of the PK set was identical to that of the safety set.

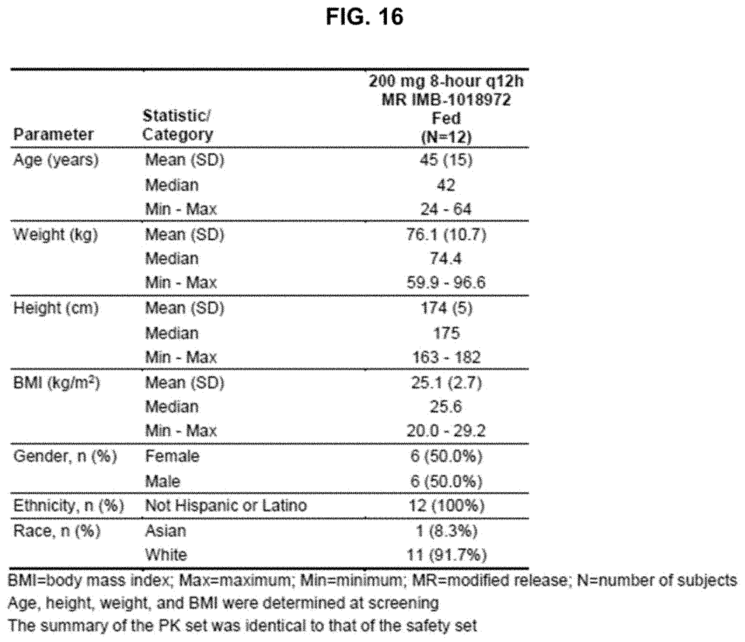

FIG. 16 is a table of a summary of demographic characteristics—Multiple-Dose MR Part (Safety Set).

Other Baseline Characteristics

All subjects complied with the inclusion and exclusion criteria. There were no clinically significant findings with regard to medical history or previous medication. Drug and alcohol screen results were negative for all subjects at screening and (each) admission. The results for the serology parameters were negative at screening for all subjects. The pregnancy test results were negative at screening, (each) admission, and follow-up for all females participating in this study.

Extent of Exposure

A total of 88 subjects were dosed in this study: 12 subjects in the single-dose MR part, and 12 subjects in the multiple-dose MR part.

In the single-dose MR part, all 12 subjects received 4 single doses of an MR formulation of IMB-1018972 under fasted conditions: 50 mg 8-hour MR formulation on Day 1, 50 mg 4-hour MR formulation on Day 4, 200 mg 8-hour MR formulation on Day 7, and 200 mg 4-hour MR formulation on Day 10. On Day 13, all 11 of 12 subjects received the 200 mg 8-hour MR formulation of IMB-1018972 under fed conditions. Subject 505 was withdrawn from the study due to a moderate TEAE of ALT increased (possibly related; up to 149 IU/L on Day 11) and did not receive the last single oral dose of 200 mg 8-hour MR formulation of IMB-1018972 under fed conditions on Day 13.

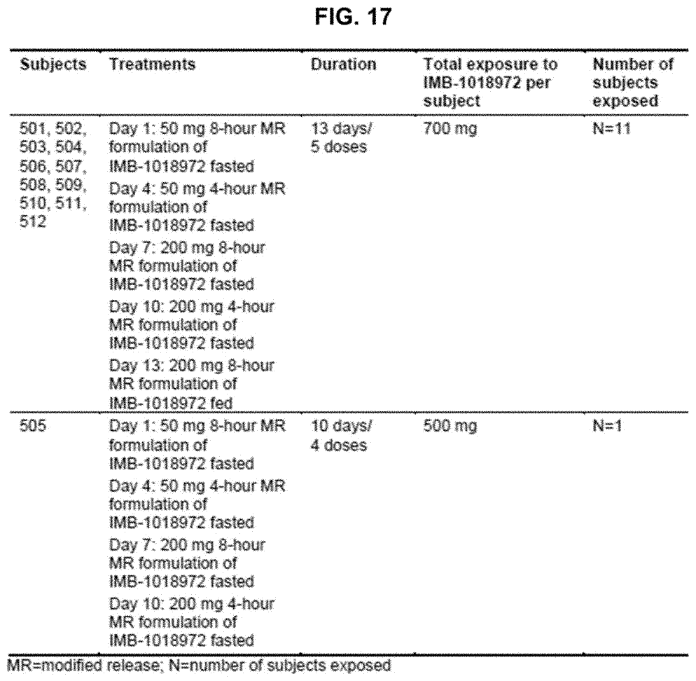

FIG. 17 is a table of the Extent of Exposure—Single-Dose MR Part (Safety Set)

In the multiple-dose MR part, all 12 subjects received multiple oral doses of the 200 mg 8-hour MR formulation of IMB-1018972 q12h under fed conditions from Day 1 to Day 4 followed by a single morning dose on Day 5.

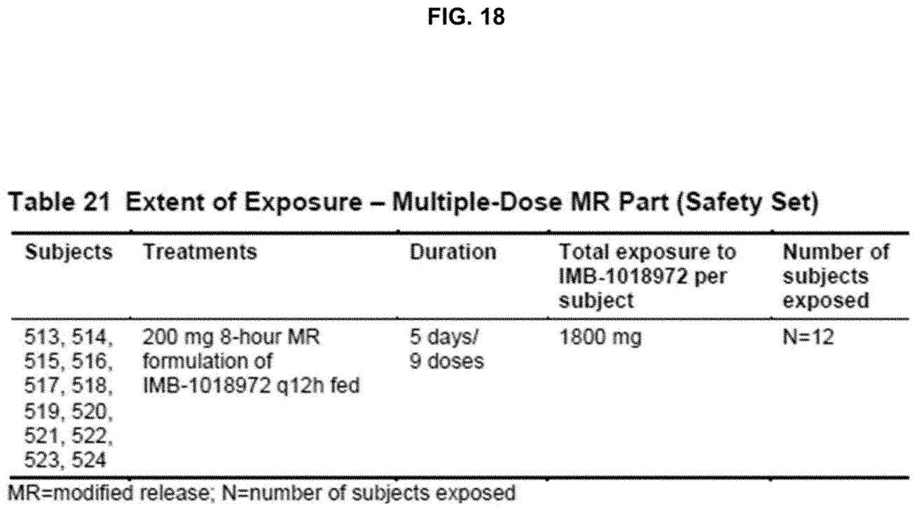

FIG. 18 is a table of the Extent of Exposure—Multiple-Dose MR Part (Safety Set)

Single-Dose MR Part

Following administration of both the 50-mg and 200-mg single oral fasted doses of IMB-1018972, tₘₐₓ for IMB-1028814 was earlier with the 8-hour MR formulation (2 hours for 50 mg and 200 mg IMB-1018972) than with the 4-hour MR formulation (5 hours for 50 mg IMB-1018972 and 3 hours for 200 mg IMB-1018972). Following administration of both the 50-mg and 200-mg single oral fasted doses of IMB-1018972, tmax for trimetazidine was later with the 8-hour MR formulation (8 hours for 50 mg IMB-1018972 and 5 hours for 200 mg IMB-1018972) than with the 4-hour MR formulation (6 hours for 50 mg IMB-1018972 and 3 hours for 200 mg IMB-1018972). Following administration of both the 50-mg and 200-mg single oral fasted doses of IMB-1018972, tmax for IMB-1028814+trimetazidine was similar for the 8-hour MR formulation (5 hours for 50 mg IMB-1018972 and 2.5 hours for 200 mg IMB-1018972) and the 4-hour MR formulation (5 hours for 50 mg IMB-1018972 and 3 hours for 200 mg IMB-1018972).

Following administration of the 50-mg and 200-mg single oral fasted doses of IMB-1018972, Cmax for IMB-1028814 was 35% and 32% lower, respectively, Cmax for trimetazidine was 20% and 24% lower, respectively, and Cmax for IMB-1028814+trimetazidine was 21% and 34% lower, respectively, for the 8-hour MR formulation relative to the 4-hour MR formulation (Table 28).

Following administration of the 50-mg single oral fasted dose of IMB-1018972, AUC₀₋ₜ for IMB-1028814 was 26% lower, Cₘₐₓ for trimetazidine was 12% lower, and Cₘₐₓ for IMB-1028814+trimetazidine was 18% lower after the 8-hour MR formulation than after the 4-hour MR formulation. Following the 200-mg single oral fasted dose of IMB-1018972, AUC0-t for IMB-1028814 was 6% higher, Cₘₐₓ for trimetazidine was 4% higher, and Cₘₐₓ for IMB-1028814+trimetazidine was 5% higher after the 8-hour MR formulation than after the 4-hour MR formulation.

Following administration of the 50-mg MR formulation and 200 mg MR formulation with 4-hour and 8-hour dissolution profile under fasted conditions, geometric mean t_{1/2} ranged between 3.35 hours and 4.27 hours for IMB-1028814, between 8.11 hours and 9.35 hours for trimetazidine, and between 6.95 hours and 7.96 hours for IMB-1028814+trimetazidine. Thus, for each of the analytes, no difference was observed in t½ was between the 4 fasted treatments.

### Effect of Food

The possible effect of food on the PK of IMB-1028814 and trimetazidine was explored by comparing administration of single oral doses of 200 mg MR formulation of IMB-1018972 with 8-hour dissolution profile after an FDA-defined high-fat breakfast and under fasted conditions.

After study drug administration under fed conditions, the geometric mean IMB-1028814 plasma concentrations initially increased less rapidly than after study drug administration under fasted conditions. However, median tₘₐₓ was reached at 3 hours postdose under both conditions.

The trimetazidine plasma concentrations under fed conditions increased less rapidly than after study drug administration under fasted conditions. Median tₘₐₓ was reached at 5 hours postdose relative to 3 hours postdose under fasted conditions.

The effect of food of IMB-1028814 and trimetazidine was explored for Cₘₐₓ, AUC₀₋ₜ, and AUC_{0-inf}. No evidence for an effect of food was observed on the IMB-1028814 exposure parameters AUC₀₋ₜ and AUC_{0-inf} (both with an estimate of 1.16 and 90% CI ranging from 1.05 to 1.28). However, Cₘₐₓ was approximately 42% higher following administration of a single dose of 200 mg 8-hour MR IMB-1018972 after an FDA-defined high-fat breakfast relative to administration under fasted conditions (estimate of 1.42; 90% CI ranging from 1.24 to 1.63).

No evidence for an effect of food was observed on the trimetazidine exposure parameters Cₘₐₓ (estimate of 1.10; 90% CI ranging from 0.99 to 1.21), AUC₀₋ₜ (estimate of 0.99; 90% CI ranging from 0.91 to 1.09), and AUC_{0-inf} (estimate of 0.97; 90% CI ranging from 0.88 to 1.07) following administration of a single dose of 200 mg 8-hour MR IMB-1018972.

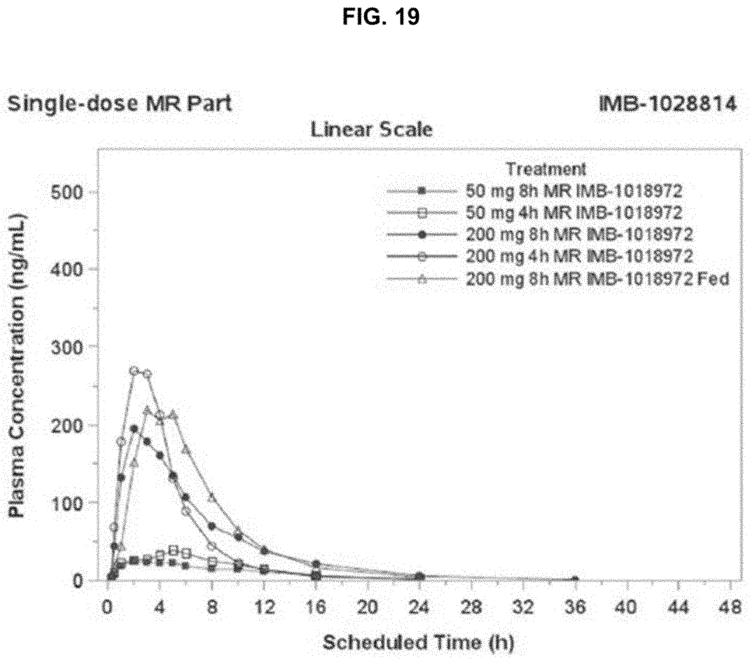

FIG. 19 is a graph of Geometric Mean IMB-1028814 Plasma Concentration-Time Profiles (Linear)—Single-Dose MR Part (PK Set)

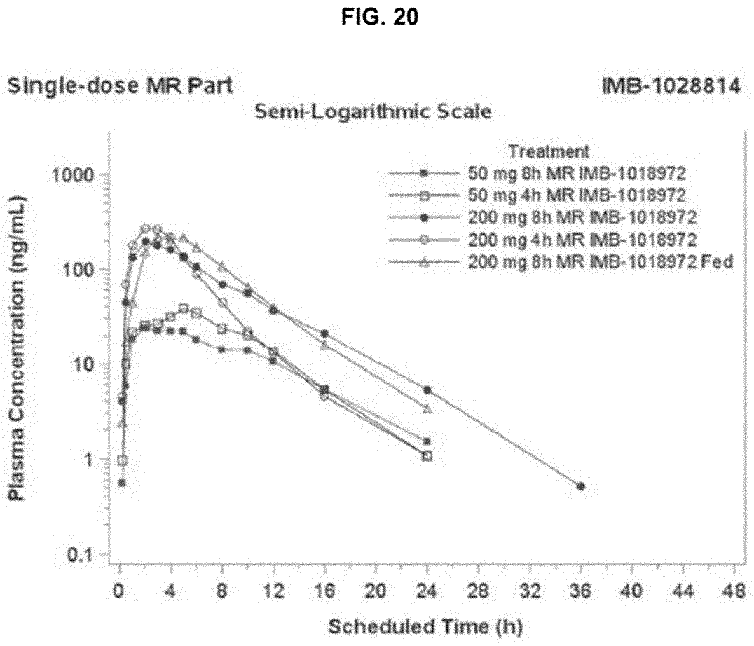

FIG. 20 is a graph of Geometric Mean IMB-1028814 Plasma Concentration-Time Profiles (Semi-Logarithmic Scale)—Single-Dose MR Part (PK Set)

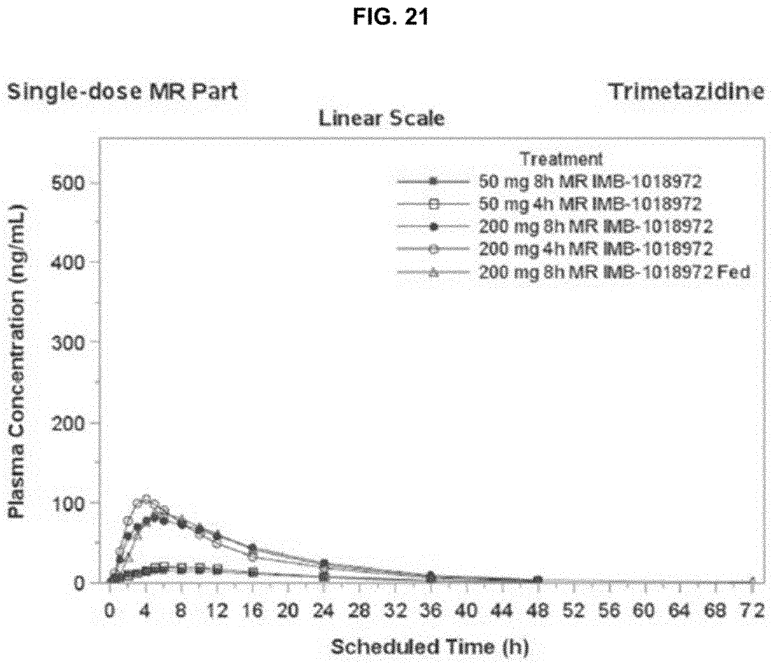

FIG. 21 is a graph of Geometric Mean Trimetazidine Plasma Concentration-Time Profiles (Linear)—Single-Dose MR Part (PK Set)

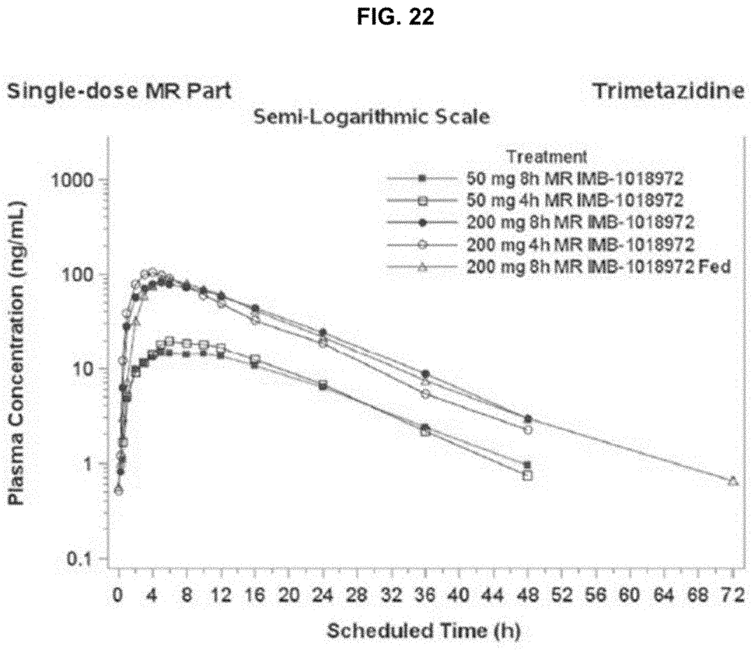

FIG. 22 is a graph of Geometric Mean Trimetazidine Plasma Concentration-Time Profiles (Semi-Logarithmic Scale)—Single-Dose MR Part (PK Set)

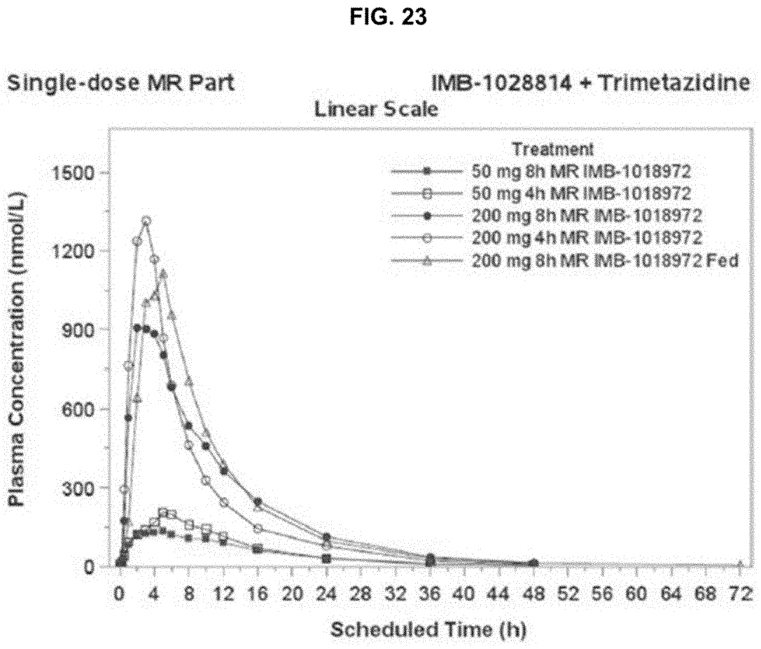

FIG. 23 is a graph of Geometric Mean IMB-1028814+Trimetazidine Plasma Concentration-Time Profiles (Linear)—Single-Dose MR Part (PK Set)

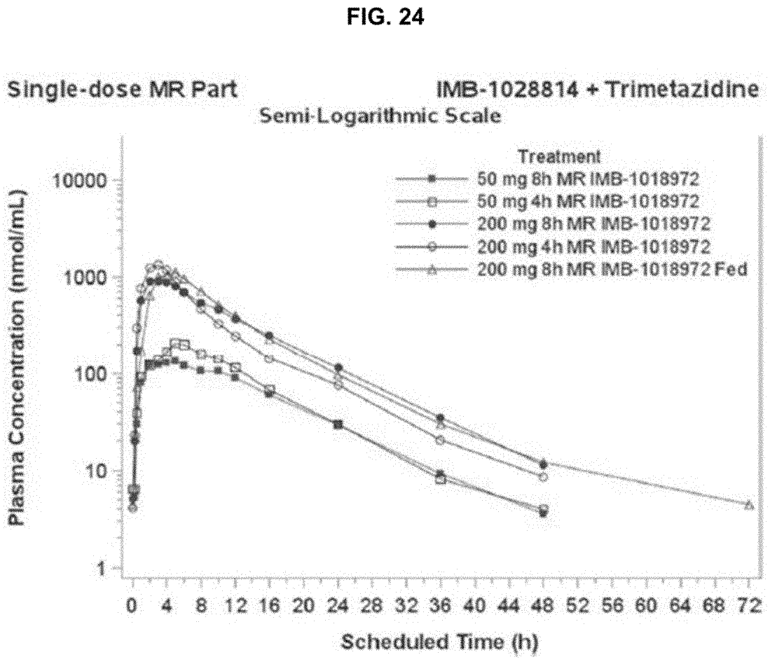

FIG. 24 is a graph of Geometric Mean IMB-1028814+Trimetazidine Plasma Concentration-Time Profiles (Semi-Logarithmic Scale)—Single-Dose MR Part (PK Set)

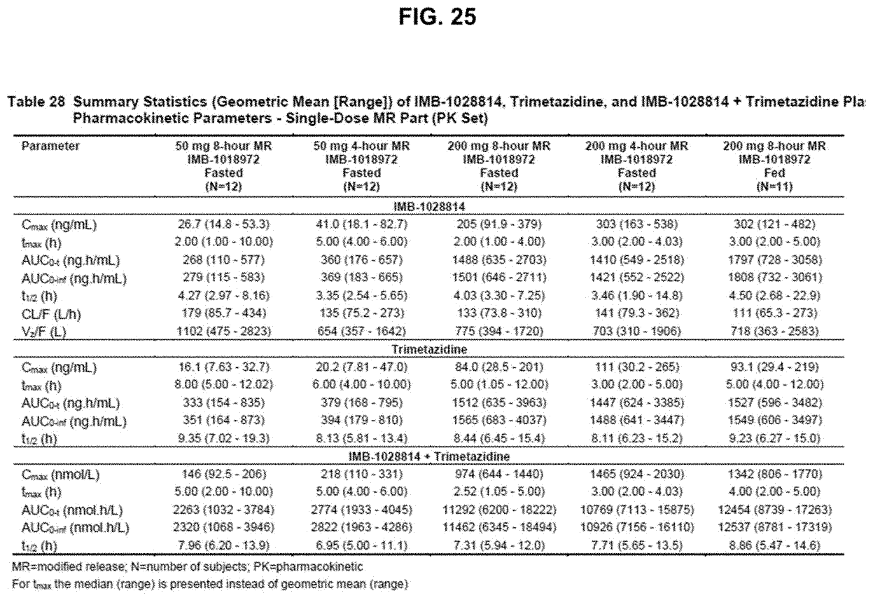

FIG. 25 is a table of Summary Statistics Geometric Mean [Range]) of IMB-1028814, Trimetazidine, and IMB-1028814+Trimetazidine Plasma Pharmacokinetic Parameters—Single-Dose MR Part (PK Set)

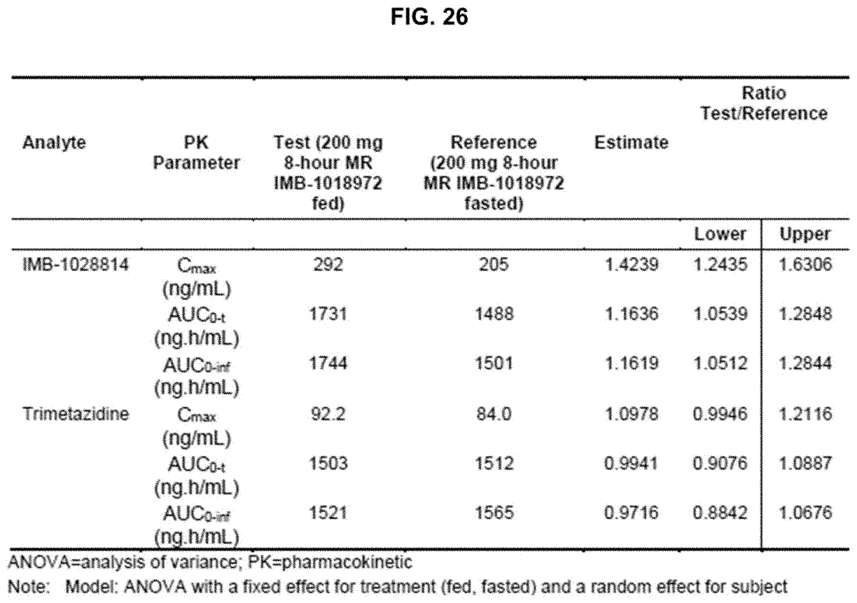

FIG. 26 is a table of Exploratory Analysis of Food Effect for IMB-1028814 and Trimetazidine following Administration of 200 mg 8-hour MR IMB-1018972—Single-Dose MR

Part (PK Set)

Multiple-Dose MR Part

All predose samples on Day 1 were below the LLOQ for IMB-1028814 and trimetazidine plasma concentrations.

Similar to the SAD and MAD parts, initial hydrolysis of IMB-1018972 to IMB-1028814 and subsequent systemic bioavailability of IMB-1028814 of the 200 mg 8-hour MR IMB-1018972 dose on Days 1 and 5 was relatively rapid. Median IMB-1028814 tₘₐₓ was 2 hours on Day 1 and Day 5, and median tₘₐₓ was 5.5 hours and 5 hours for trimetazidine on Day 1 and Day 5, respectively.

Based upon visual inspection of the geometric mean plasma concentration-time profiles and the geometric mean trough concentrations, it can be concluded that steady state for both IMB-1028814 and trimetazidine concentrations was reached by Day 5 following multiple dose administration of 200 mg 8-hour MR IMB-1018972.

Geometric mean Rac for IMB-1028814, trimetazidine, and IMB-1028814+trimetazidine were 1.22, 2.28, and 1.66 on Day 5 relative to Day 1. This indicates minimal accumulation of IMB-1028814 in plasma, moderate accumulation of trimetazidine in plasma, and moderate accumulation of IMB-1028814+trimetazidine in plasma.

The geometric mean half-life of the 200 mg 8-hour MR IMB-1018972 dose was 3.85 hours, 9.52 hours, and 8.64 hours for IMB-1028814, trimetazidine, and IMB-1028814+trimetazidine, respectively.

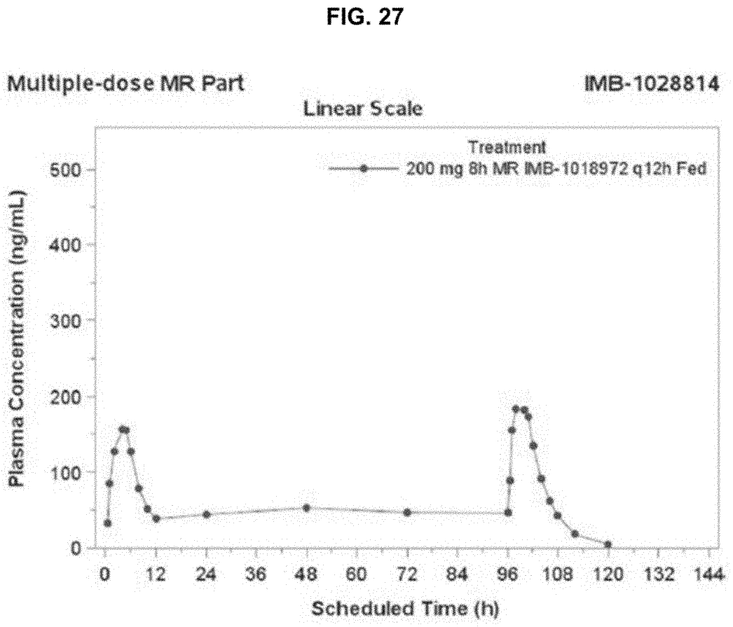

FIG. 27 is a graph of Geometric Mean IMB-1028814 Plasma Concentration-Time Profiles from Day 1 through Day 5 (Linear)—Multiple-Dose MR Part (PK Set)

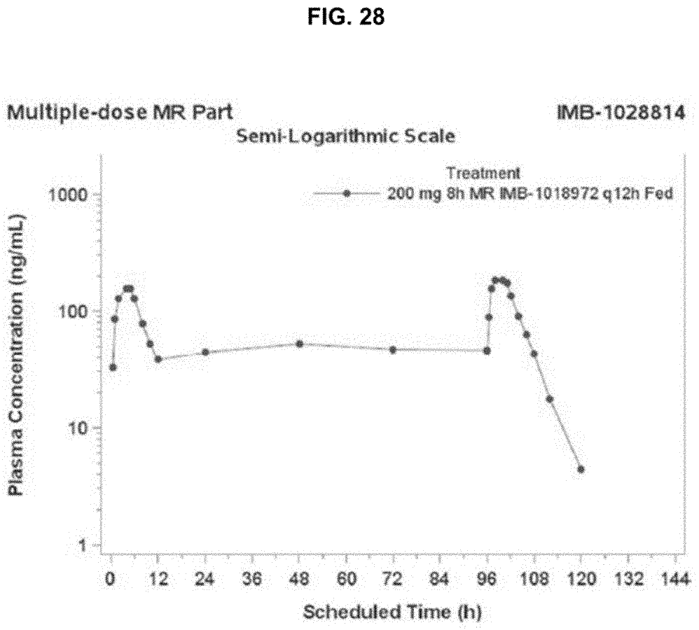

FIG. 28 is a graph of Geometric Mean IMB-1028814 Plasma Concentration-Time Profiles from Day 1 through Day 5 (Semi-Logarithmic Scale)—Multiple-Dose MR Part (PK Set)

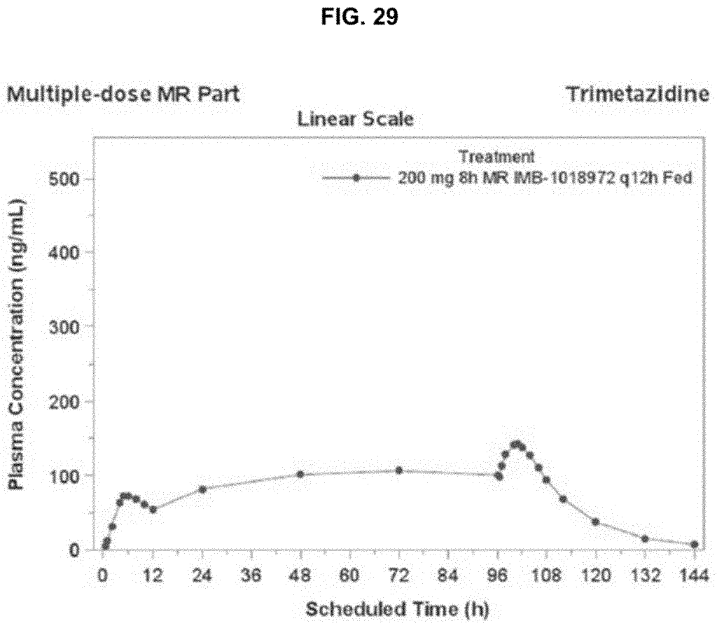

FIG. 29 is a graph of Geometric Mean Trimetazidine Plasma Concentration-Time Profiles from Day 1 through Day 5 (Linear)—Multiple-Dose MR Part (PK Set)

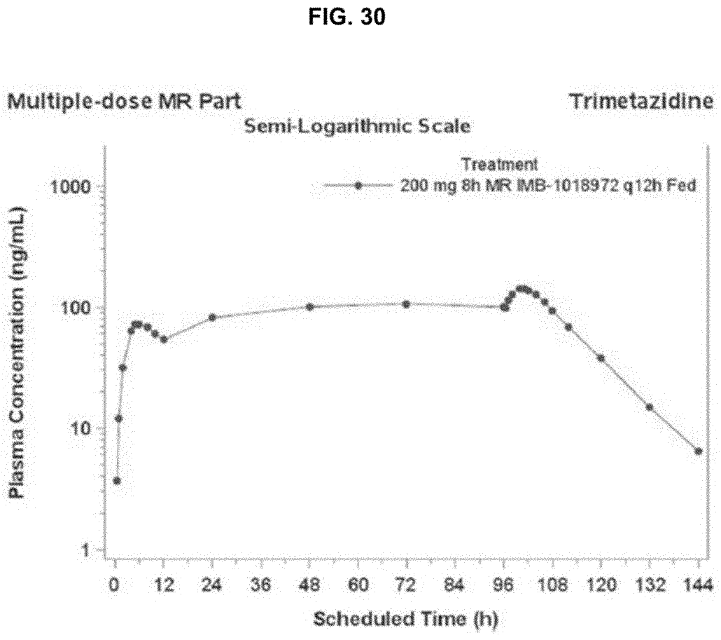

FIG. 30 is a graph of Geometric Mean Trimetazidine Plasma Concentration-Time Profiles from Day 1 through Day 5 (Semi-Logarithmic Scale)—Multiple-Dose MR Part (PK Set)

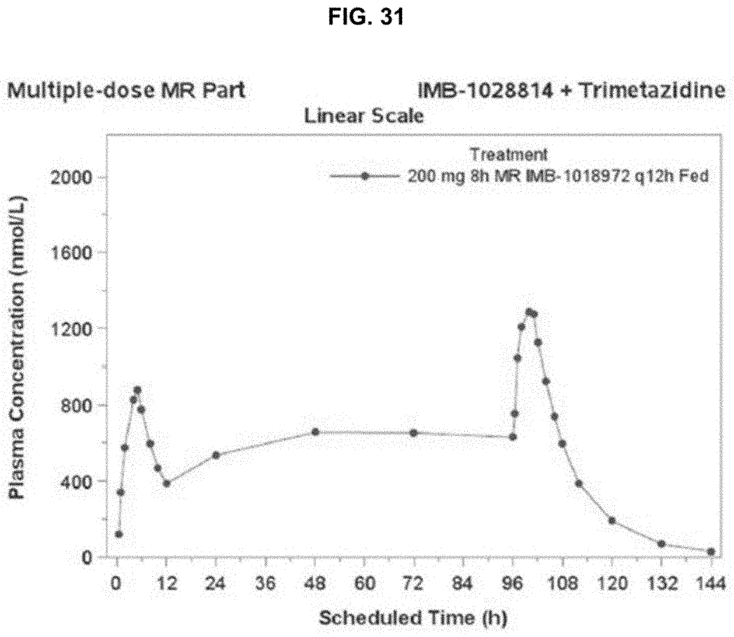

FIG. 31 is a graph of Geometric Mean IMB-1028814+Trimetazidine Plasma Concentration-Time Profiles from Day 1 through Day 5 (Linear)—Multiple-Dose MR Part (PK Set)

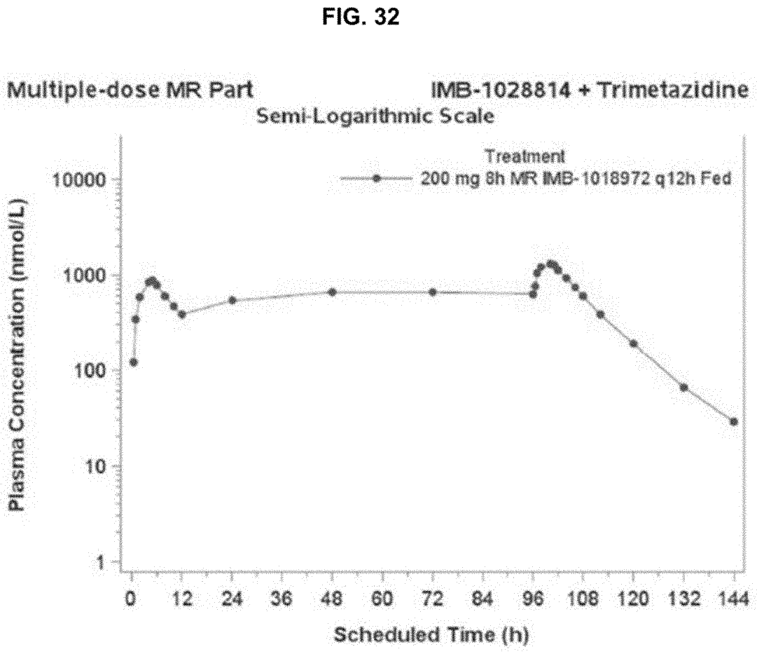

FIG. 32 is a graph of Geometric Mean IMB-1028814+Trimetazidine Plasma Concentration-Time Profiles from Day 1 through Day 5 (Semi-Logarithmic Scale)—Multiple-Dose MR Part (PK Set)

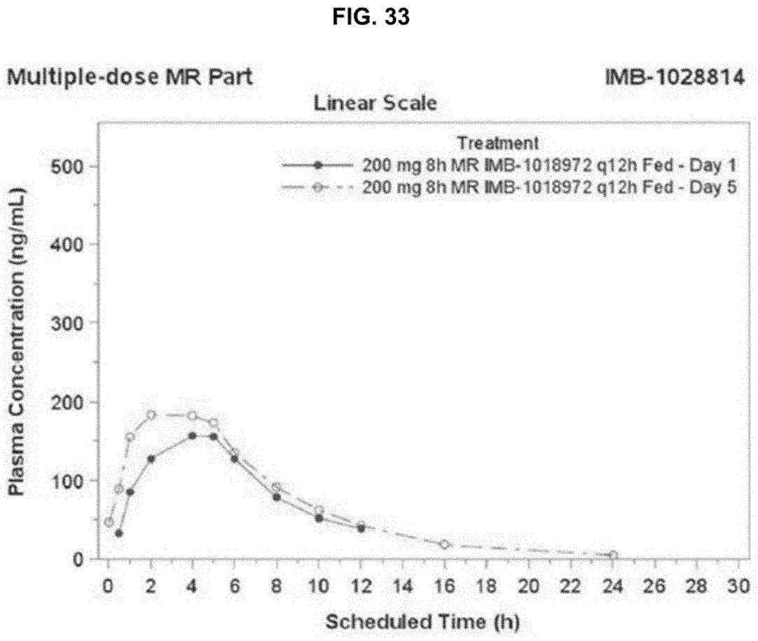

FIG. 33 is a graph of Geometric Mean IMB-1028814 Plasma Concentration-Time Profiles after Day 1 through Day 5 (Linear)—Multiple-Dose MR Part (PK Set)

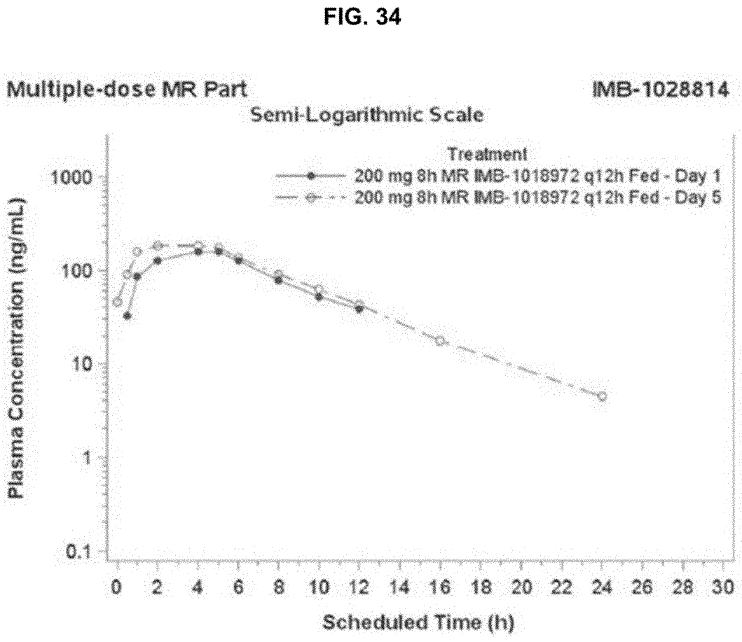

FIG. 34 is a graph of Geometric Mean IMB-1028814 Plasma Concentration-Time Profiles after Day 1 through Day 5 (Semi-Logarithmic Scale)—Multiple-Dose MR Part (PK Set)

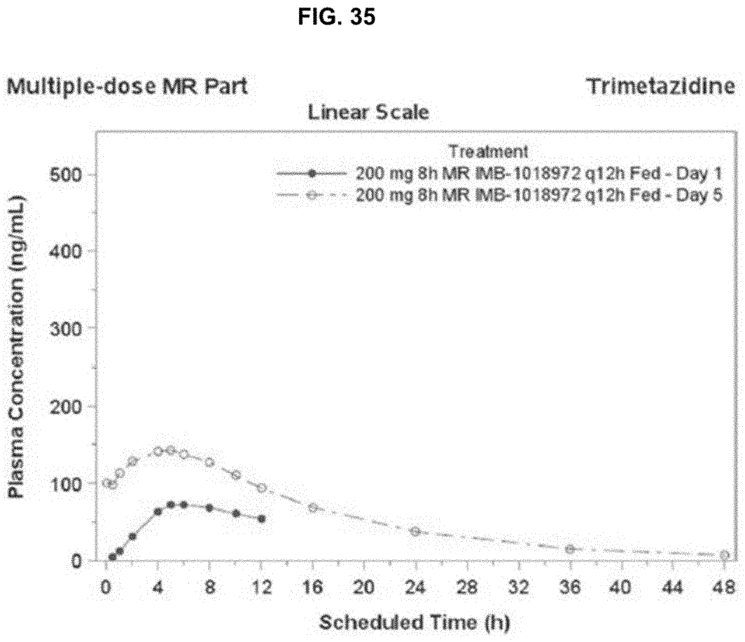

FIG. 35 is a graph of Geometric Mean Trimetazidine Plasma Concentration-Time Profiles after Day 1 through Day 5 (Linear)—Multiple-Dose MR Part (PK Set)

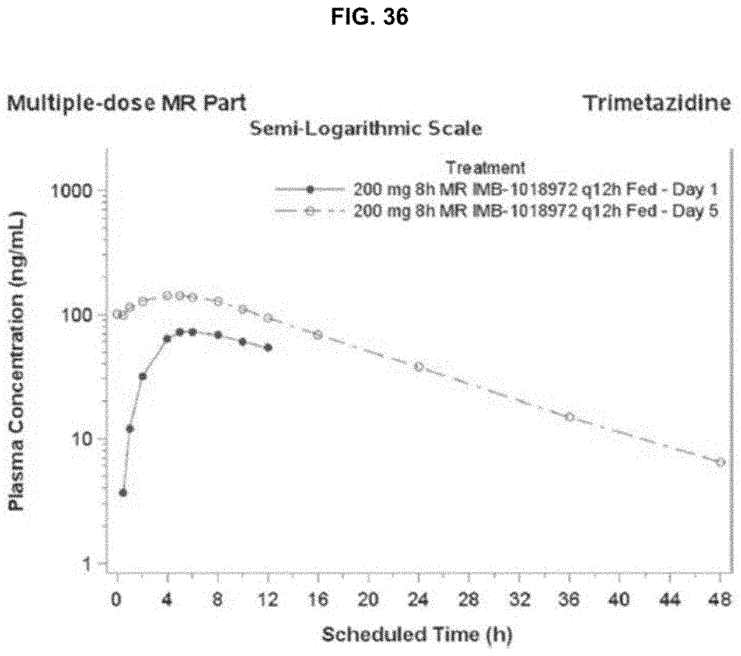

FIG. 36 is a graph of Geometric Mean Trimetazidine Plasma Concentration-Time Profiles after Day 1 through Day 5 (Semi-Logarithmic Scale)—Multiple-Dose MR Part (PK Set)

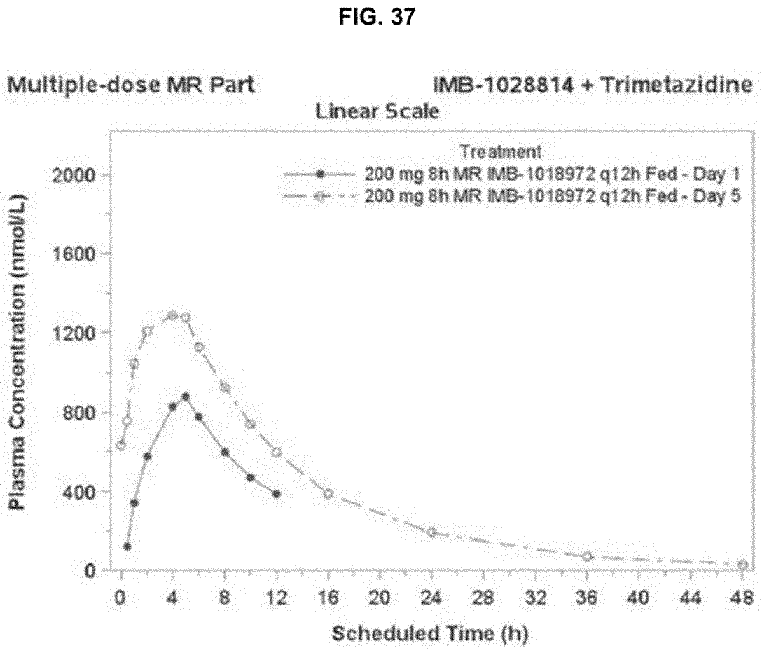

FIG. 37 is a graph of Geometric Mean IMB-1028814+Trimetazidine Plasma Concentration-Time Profiles after Day 1 through Day 5 (Linear)—Multiple-Dose MR Part (PK Set)

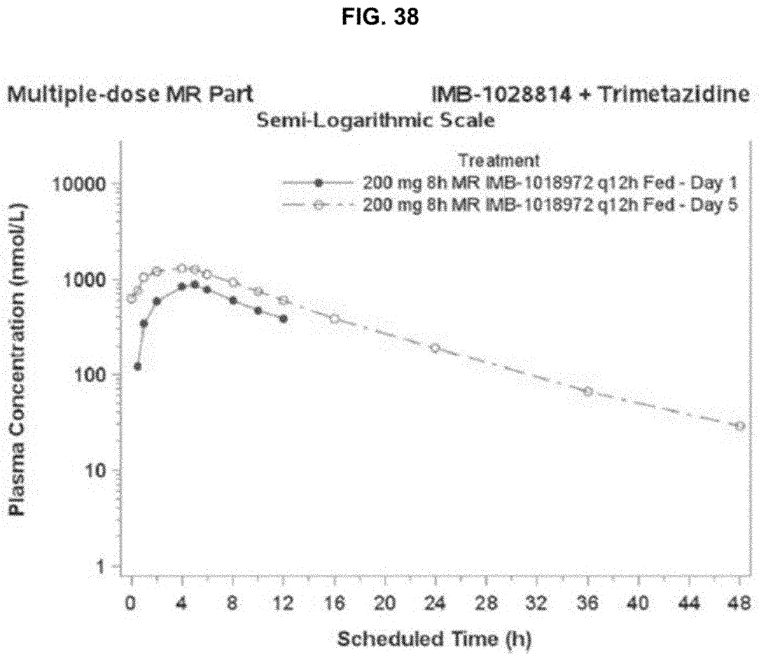

FIG. 38 is a graph of Geometric Mean IMB-1028814+Trimetazidine Plasma Concentration-Time Profiles after Day 1 through Day 5 (Semi-Logarithmic Scale)—Multiple-Dose MR Part (PK Set)

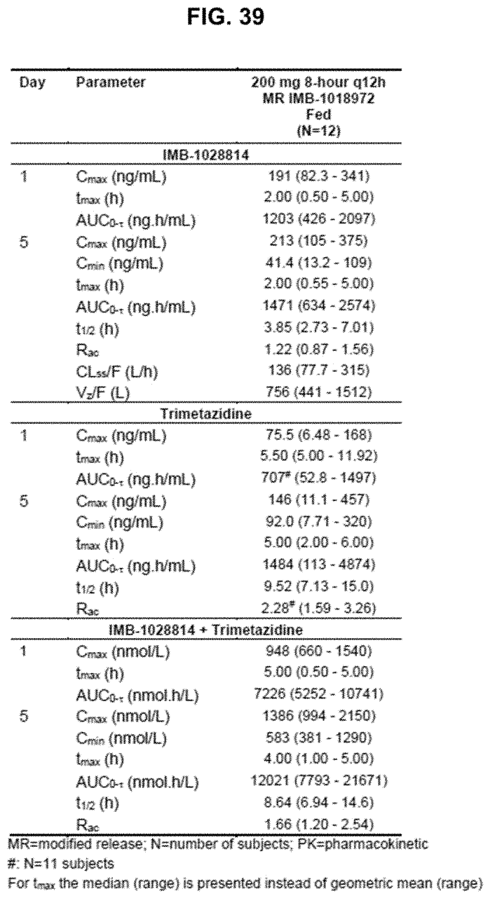

FIG. 39 is a table of Summary Statistics Geometric Mean [Range]) of IMB-1028814, Trimetazidine, and IMB-1028814+Trimetazidine Plasma Pharmacokinetic Parameters—Multiple-Dose MR Part (PK Set)

Conclusion on Pharmacokinetics
IMB-1018972 could be measured in only few plasma samples taken during this study.
When combining the single and multiple IMB-1018972 dose results under fasted and fed conditions, including those of the MR formulations, the initial hydrolysis of IMB-1018972 to IMB-1028814 and subsequent systemic bioavailability of IMB-1028814 was relatively rapid with median tₘₐₓ ranging between 0.5 hours and 5 hours postdose for IMB-1028814, and between 1.5 hours and 8 hours postdose for trimetazidine. Median tₘₐₓ did not increase with increasing IMB-1018972 dose.
The predefined stopping criterion for IMB-1028814 plasma exposure of 417,733 and 652,849 ng*h/mL for males and females, respectively, was not reached by any of the subjects during the SAD part or MAD part.
Following single oral IMB-1018972 doses in the range of 50 to 400 mg under fasted conditions, systemic exposure to IMB-1028814 and trimetazidine was dose proportional for Cₘₐₓ, AUC₀₋ₜ, and AUC_{0-inf}.
No evidence for an effect of food was observed on the IMB-1028814 exposure parameters AUC₀₋ₜ and AUC_{0-inf} following administration of a single dose of 150 mg IMB-1018972. However, Cₘₐₓ was approximately 36% lower following administration of a single dose of 150 mg IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 150 mg IMB-1018972.
No evidence for an effect of food was observed on the IMB-1028814 exposure parameters AUC₀₋ₜ and AUC_{0-inf} following administration of a single dose of 200 mg 8-hour MR IMB-1018972. However, Cₘₐₓ was approximately 42% higher following administration of a single dose of 200 mg 8-hour MR IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 200 mg 8-hour MR IMB-1018972.
When combining the single and multiple IMB-1018972 dose results under fasted and fed conditions, including those of the MR formulations, the geometric mean t_{1/2} ranged between 2.5 hours and 4.5 hours for IMB-1028814, and between 6.5 hours and 9.5 hours for trimetazidine. Geometric mean t½ did not increase with increasing IMB-1018972 dose.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted in urine as trimetazidine.
Within 48 hours following administration of a single oral dose of 35 mg trimetazidine, on average 54.47% of the dose was excreted in urine as trimetazidine. □Following 14 days of twice daily dosing with 150 mg and 50 mg IMB-1018972 under fed conditions, no relevant accumulation was observed of IMB-1028814 (R_{ac} of 1.18 and 1.10 for 150 mg and 50 mg, respectively) and accumulation of trimetazidine was modest (R_{ac} of 1.63 and 1.89 for 150 mg and 50 mg, respectively) was observed. Following 5 days of twice daily dosing with 200 mg 8-hour MR IMB-1018972 under fed conditions, no relevant accumulation of IMB-1028814 (R_{ac} of 1.22) was observed, whereas accumulation of trimetazidine was moderate (R_{ac} of 2.28).

Summary of Adverse Events

### Single-Dose MR Part

A total of 37 TEAEs was reported by 10 of 12 (83.3%) subjects who received IMB-1018972. There were no deaths reported. Subject 505 of the single-dose MR part was withdrawn from the study due to a moderate TEAE of ALT increased. This TEAE is described below and more extensively in Section 12.2.2. The majority of the TEAEs were transient and resolved without sequelae by follow-up. Three TEAEs were still ongoing at follow-up: aphthous ulcer, catheter site hematoma, and catheter site related reaction. Thirty-six of 37 TEAEs were of mild severity and 1 TEAE was of moderate severity. No severe TEAEs were reported. The moderate TEAE was an event of ALT increased (up to 149 IU/L on Day 11) that was considered by the Investigator not to be related to the study drug. The subject (Subject 505) was withdrawn from the study due to this TEAE and did not receive the last single oral dose of 200 mg 8-hour MR formulation of IMB-1018972 under fed conditions on Day 13.

Of 37 TEAEs, 6 were reported by 4 (33.3%) subjects receiving the 50 mg 8-hour MR formulation of IMB-1018972 under fasted conditions, 9 were reported by 6 (50%) subjects receiving the 50 mg 4-hour MR formulation of IMB-1018972 under fasted conditions, 6 were reported by 5 (41.7%) subjects receiving the 200 mg 8-hour MR formulation of IMB-1018972 under fasted conditions, 11 were reported by 8 (66.7%) subjects receiving the 200 mg 4-hour MR formulation of IMB-1018972 under fasted conditions, and 5 were reported by 2 (18.2%) subjects receiving the 200 mg 8-hour MR formulation of IMB-1018972 under fed conditions. There was no apparent dose or dissolution time dependency of the number and incidence of TEAEs. Neither was there any clear difference between fasted and fed IMB-1018972 administration of the 200 mg 8-hour MR formulation of IMB-1018972 for the number and incidence of TEAEs.

The most frequently reported TEAEs (i.e., reported by ≥20% of the subjects) by SOC were:
General disorders and administration site conditions with 11 TEAEs reported by 7 (58.3%) subjects (5 TEAEs of catheter site related reaction, 2 TEAEs of catheter site hematoma, and 1 TEAE each of catheter site pain, malaise, medical device site dryness, and medical device site irritation.
Nervous system disorders with 8 TEAEs reported by 7 (58.3%) subjects (6 TEAEs of headache and 2 TEAEs of dizziness).
Gastrointestinal disorders with 5 TEAEs reported by 4 (33.3%) subjects (2 TEAEs of abdominal pain and 1 TEAE each of abdominal pain upper, aphthous ulcer, and diarrhea).
   Of 37 TEAEs reported, 7 TEAEs reported by 4 of 12 (33.3%) subjects were considered by the Investigator to be related to the study drug and 30 TEAEs reported by 10 of 12 (83.3%) subjects were considered by the Investigator not to be related to the study drug. No drug-related TEAEs were reported following the 200 mg 8-hour MR formulation of IMB-1018972 under fed conditions. The reported drug-related TEAEs were:
Renal and urinary disorders with 5 TEAEs of pollakiuria in 2 (16.7%) subjects.
Investigations with 1 TEAE of ALT increased.
Nervous system disorders with 1 TEAE of headache.

Overall Tolerability

Treatment with the 50 mg MR formulation and 200 mg MR formulation with 4-hour and 8-hour dissolution profile under fasted conditions, and subsequently, the 200 mg 8-hour MR formulation of IMB-1018972 under fed conditions were well tolerated by healthy male and female subjects, except for 1 subject in which ALT was elevated (up to 149 IU/L) after 2 single doses of 50 mg MR formulation and 2 doses of 200 mg MR formulation.

### Multiple-Dose MR Part

A total of 40 TEAEs was reported by 12 of 12 (100%) subjects who received IMB-1018972. All TEAEs were of mild severity and there were no deaths reported. The majority of the TEAEs were transient and resolved without sequelae by follow-up. Four TEAEs were still ongoing at follow-up: dermatitis contact, erythema, influenza like illness, oropharyngeal pain, and medical device site irritation.

The most frequently reported TEAEs (i.e., reported by ≥30% of the subjects) by SOC were:
Nervous system disorders with 9 TEAEs reported by 7 (58.3%) subjects (6 TEAEs of headache, 2 TEAEs of dizziness, and 1 TEAE of dizziness postural).
Gastrointestinal disorders with 9 TEAEs reported by 5 (41.7%) subjects (2 TEAEs of abdominal pain and 1 TEAE each of diarrhea, dyspepsia, feces pale, flatulence, gingival pain, oral discomfort, and toothache).
General disorders and administration site conditions with 4 TEAEs reported by 4 (33.3%) subjects (1 TEAE each of catheter site pain, influenza like illness, medical device site irritation, and thirst.
Musculoskeletal and connective tissue disorders with 4 TEAEs reported by 4 (33.3%) subjects (2 TEAEs of myalgia, and 1 TEAE each of neck pain and pain in extremity.
Renal and urinary disorders with 4 TEAEs of pollakiuria in 4 (33.3%) subjects.

Of 40 TEAEs reported, 10 TEAEs reported by 7 of 12 (58.3%) subjects were considered by the Investigator to be related to the study drug and 30 TEAEs reported by 9 of 12 (75%) subjects were considered by the Investigator not to be related to the study drug. The most frequently reported drug-related TEAEs (i.e., reported by ≥15% of the subjects) by SOC were:
Renal and urinary disorders with 4 TEAEs of pollakiuria in 4 (33.3%) subjects.
Nervous system disorders with 2 TEAEs reported by 2 (16.7%) subjects (1 TEAE each of dizziness and headache).
Vascular disorders with 2 TEAEs of flushing reported by 2 (16.7%) subjects.

Overall Tolerability

Five-day treatment with multiple oral doses of the 200 mg 8-hour MR formulation of IMB-1018972 q12h under fed conditions was well tolerated by healthy male and female subjects. Of note, 2 instances of flushing of mild severity were reported by 2 subjects, Subjects 513 and 517, who were post-menopausal females and 1 of whom had reported ongoing “hot flushes” as part of medical history. No subjects dropped out and no modification of the dose was needed due to the TEAEs of flushing.

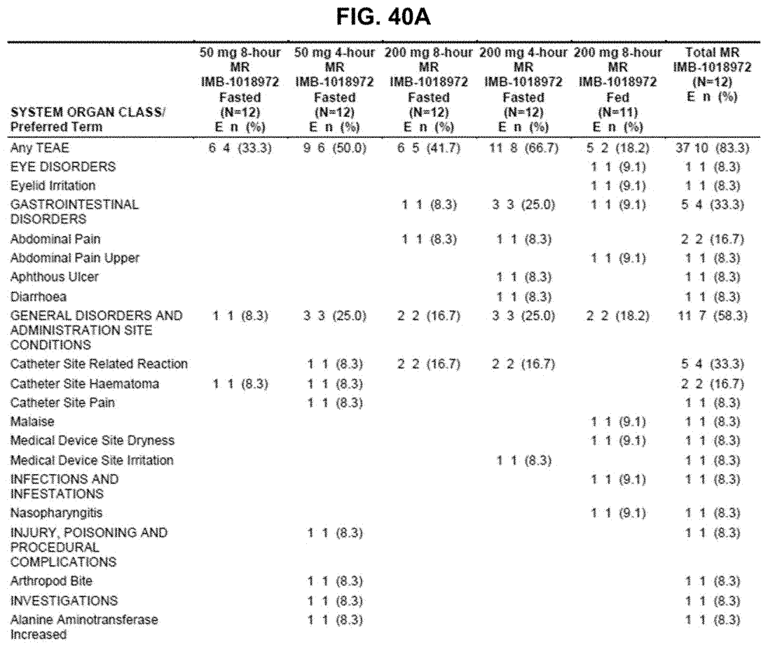

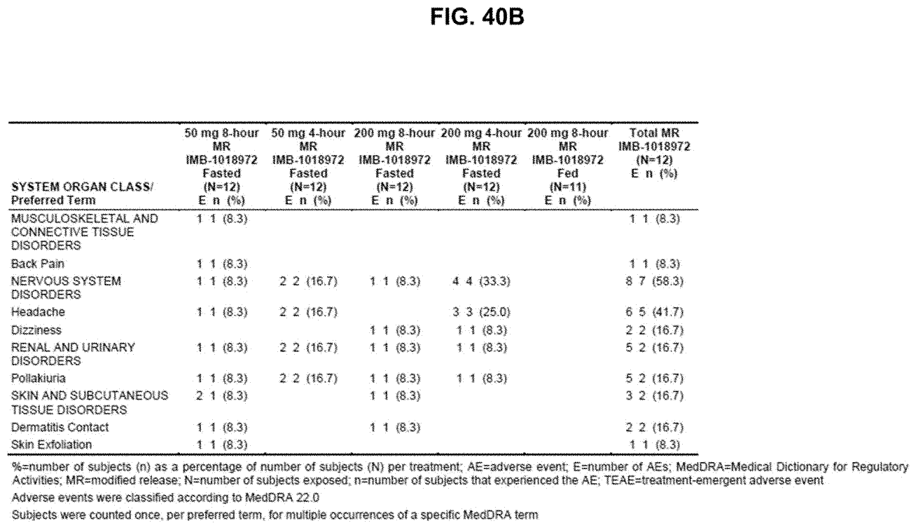

FIG. 40A and FIG. 40B is a table Summary of All TEAEs by System Organ Class, Preferred Term and Treatment—Single-Dose MR Part (Safety Set)

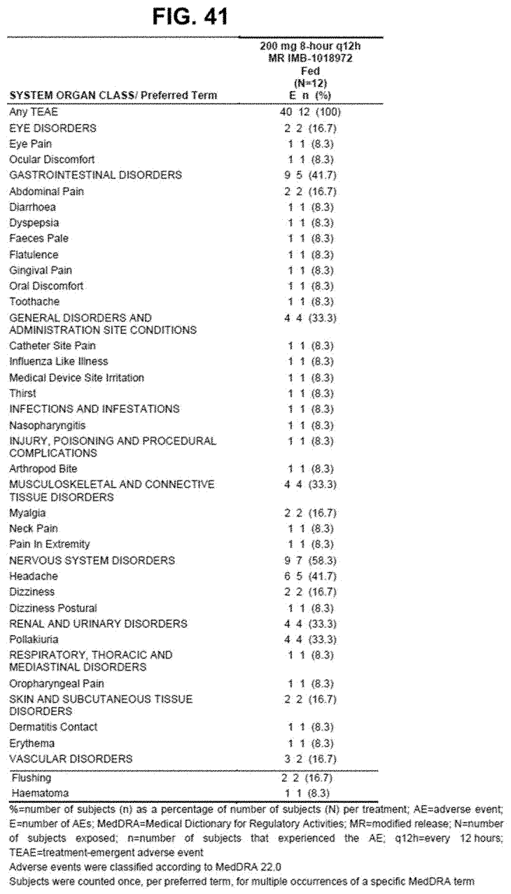

FIG. 41 is a table Summary of All TEAEs by System Organ Class, Preferred Term and Treatment—Single-Dose MR Part (Safety Set)

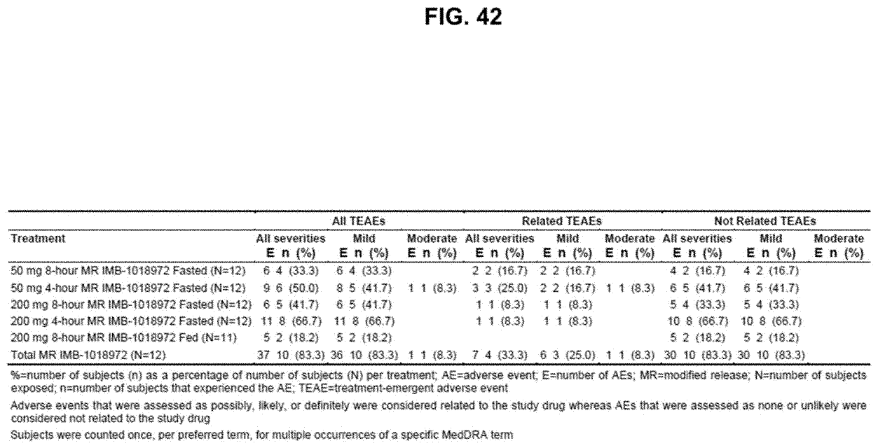

FIG. 42 is a table Summary of All TEAEs by Treatment, Relationship, and Severity-Single-Dose MR Part (Safety Set)

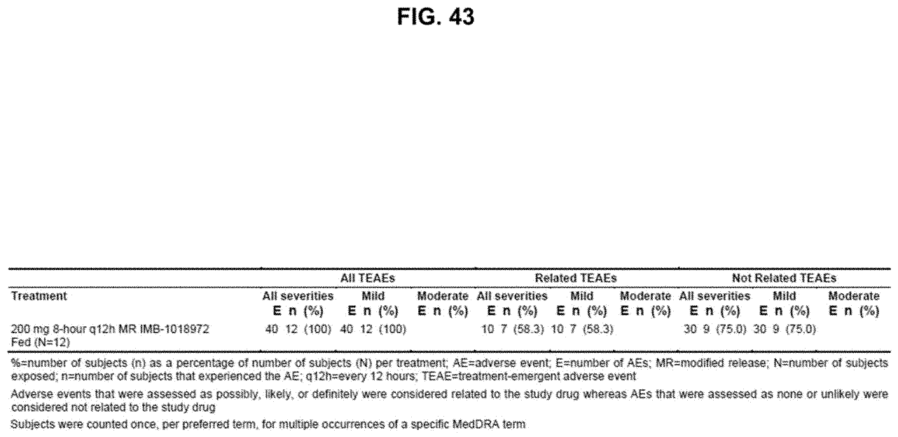

FIG. 43 is a table Summary of All TEAEs by Treatment, Relationship, and Severity—Multiple-Dose MR Part (Safety Set)

### Deaths, Other Serious Adverse Events, and Other Significant Adverse Events

One subject was withdrawn during the study.

Subject 505 was a 21-year old white male with a BMI of 21.5 kg/m2. The subject participated in the single-dose MR part and was planned to receive 50 mg of the 8-hour MR formulation on Day 1 under fasted conditions, 50 mg of the 4-hour MR formulation on Day 4 under fasted conditions, 200 mg of the 8-hour MR formulation on Day 7 under fasted conditions, 200 mg of the 4-hour MR formulation on Day 10 under fasted conditions, and 200 mg 8-hour MR formulation of IMB-1018972 on Day 13 under fed conditions. He reported no relevant medical history and received no concomitant medication at baseline. A TEAE of ALT increased was reported for this subject starting on Day 5, 1 day after dosing with 50 mg of the 4-hour MR formulation on Day 4. This TEAE was of moderate severity and considered by the Investigator to be possibly related to the study drug. The subject also received the doses of 200 mg of the 8-hour MR formulation on Day 7 under fasted conditions and 200 mg of the 4-hour MR formulation on Day 10 under fasted conditions. ALT values for this subject were within normal range (0-68 IU/L) at screening (29 IU/L), on Day −1 (34 IU/L), and on Day 2 (31 IU/L). ALT levels increased to values above the upper limit of normal (68 IU/L) of 72 IU/L on Day 5, 97 IU/L on Day 8, and 149 IU/L on Day 11, and then decreased again to 102 IU/L on Day 14, and 84 IU/L on Day 16. By follow-up on Day 24, ALT levels had returned to 42 IU/L, which was within the normal range. This was also the day that this TEAE was recorded to have recovered. The high ALT level of 149 IU/L on Day 11 was considered by the Investigator to be clinically significant abnormal, based on which the Investigator decided to withdraw the subject from the study (receive no further doses). Throughout this entire period, AST levels were within normal range. As a result of withdrawal, the subject did not receive the planned last single oral dose of 200 mg 8-hour MR formulation of IMB-1018972 under fed conditions on Day 13. After withdrawal on Day 11, the subject returned on Day 24 for a follow-up with safety assessments conducted as planned. The subject also reported mild TEAEs of dermatitis contact on Day 1 (not related), skin exfoliation from Day 3 to Day 6 (not related), and abdominal pain from Day 13 to Day 14 (not related).

The TEAE of ALT increased that led to the withdrawal of Subject 505 from the study was considered by the Investigator to be possibly related to the study drug due to its time-relationship with study drug administration.

Subject 505 of the single-dose MR part was withdrawn from the study due to a moderate TEAE of ALT increased. The TEAE started on Day 5, 1 day after dosing with 50 mg of the 4-hour MR formulation on Day 4. ALT values for this subject were within normal range (0-68 IU/L) at screening (29 IU/L), on Day −1 (34 IU/L), and on Day 2 (31 IU/L). ALT levels increased to values above the upper limit of normal (68 IU/L) of 72 IU/L on Day 5, 97 IU/L on Day 8, and 149 IU/L on Day 11, and then decreased again to 102 IU/L on Day 14, and 84 IU/L on Day 16. By follow-up on Day 24, ALT levels had returned to 42 IU/L, which was within the normal range. This was also the day that this TEAE was recorded to have recovered. The high ALT level of 149 IU/L on Day 11 was considered by the Investigator to be clinically significant abnormal, based on which the Investigator decided to withdraw the subject from the study. Throughout this entire period, AST levels for Subject 505 were within normal range. No other cases of clinically significant abnormal laboratory parameters were recorded at any time during this study.

### Concomitant Treatment

Single-Dose MR Part

Seven subjects in the single-dose MR part received or took concomitant medication. Five female subjects used contraception during the study. In addition, 4 subjects received concomitant medication as follows:
One subject (Subject 501) received 1000 mg paracetamol once because of headache.
One subject (Subject 509) received 1000 mg paracetamol once because of headache intermittent (preferred term: headache).
One subject (Subject 510) received 500 mg paracetamol twice and 1000 mg paracetamol once because of headache.
One subject (Subject 511) received 1000 mg paracetamol once because of common cold (preferred term: nasopharyngitis).
   These medications were not considered to have influenced the outcome of the study.

Multiple-Dose MR Part

Five subjects in the multiple-dose MR part received or took concomitant medication. Three female subjects used contraception during the study. In addition, 3 subjects received concomitant medication as follows:
One subject (Subject 514) received 1000 mg paracetamol twice because of headache.
One subject (Subject 519) received gelomyrtol 3 times a day for 2 days and 500 mg paracetamol 3 times a day for 2 days because of flu like symptoms (preferred term: influenza like illness).
One subject (Subject 524) received 500 mg paracetamol once because of headache. These medications were not considered to have influenced the outcome of the study.

### Discussion and Overall Conclusions

This was a double-blind, randomized study, consisting of single-dose and multiple-dose MR parts to assess the safety, tolerability, and PK of single oral doses of a MR formulation of trimetazidine, single oral doses of MR formulations of IMB-1018972, and multiple oral doses of the 200 mg 8-hour MR formulation of IMB-1018972.

Safety Discussion

Overall, single and multiple doses of MR formulations, were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the MR formulations.

Nicotinic acid (niacin) is an immediate hydrolysis product of IMB-1018972 and constitutes approximately 30% of the molecular mass of IMB-1018972. In this study, TEAEs of flushing, of which the characteristics were consistent with the flushing seen with the administration of niacin, were reported. All events were transient and resolved without intervention. No subjects dropped out and no modification of the dose was needed due to the TEAEs of flushing.

In the multiple-dose MR part, 2 instances of flushing of mild severity were reported by 2 subjects who were post-menopausal females and 1 of whom had reported ongoing “hot flushes” as part of medical history. No subjects dropped out and no modification of the dose was needed due to the TEAEs. The nature of these TEAEs being sporadic, transient, self-limiting, and of mild severity in the MR formulation indicate an acceptable tolerability profile of IMB-1018972 in the 200 mg 8-hour MR formulation.

One subject of the single-dose MR part was withdrawn from the study due to a moderate TEAE of ALT increased. The TEAE of ALT increased (up to 149 IU/L on Day 11) was considered by the Investigator to be possibly related to the study drug and resolved without intervention. The most frequently reported TEAEs during the study were of the SOC vascular disorders (mainly TEAEs of flushing), general disorders and administration site conditions, nervous system disorders, gastrointestinal disorders, and musculoskeletal and connective tissue disorders. The majority of the TEAEs reported during the study were considered by the Investigator not to be related to the study drug.

Pharmacokinetics

The 200 mg 8-hour MR IMB-1018972 formulation has been chosen to be most suitable to be used in Phase 2 proof-of-concept studies.

### Safety—Conclusion

Overall, single and multiple doses of MR formulations, were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 either as the IR or MR formulations.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions no clear difference between fasted and fed IMB-1018972 administration for the number and incidence of TEAEs was observed in the single-dose MR part.

### Pharmacokinetics—Conclusions

IMB-1018972 could be measured in only few plasma samples taken during this study.
When combining the single and multiple IMB-1018972 dose results under fasted and fed conditions, the initial hydrolysis of IMB-1018972 to IMB-1028814 and subsequent systemic bioavailability of IMB-1028814 was relatively rapid with median tₘₐₓ ranging between 0.5 hours and 5 hours postdose for IMB-1028814, and between 1.5 hours and 8 hours postdose for trimetazidine. Median tₘₐₓ did not increase with increasing IMB-1018972 dose.
Following single oral IMB-1018972 doses in the range of 50 to 400 mg under fasted conditions, systemic exposure to IMB-1028814 and trimetazidine was dose proportional for Cₘₐₓ, AUC₀₋ₜ, and AUC_{0-inf}.
No evidence for an effect of food was observed on the IMB-1028814 exposure parameters AUC₀₋ₜ and AUC_{0-inf} following administration of a single dose of 200 mg 8-hour MR IMB-1018972. However, Cₘₐₓ was approximately 42% higher following administration of a single dose of 200 mg 8-hour MR IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 200 mg 8-hour MR IMB-1018972.
When combining the single and multiple IMB-1018972 dose results under fasted and fed conditions, including those of the MR formulations, the geometric mean t_{1/2} ranged between 2.5 hours and 4.5 hours for IMB-1028814, and between 6.5 hours and 9.5 hours for trimetazidine. Geometric mean t_{1/2} did not increase with increasing IMB-1018972 dose.
Following 5 days of twice daily dosing with 200 mg 8-hour MR IMB-1018972 under fed conditions, no relevant accumulation of IMB-1028814 (R_{ac} of 1.22) was observed, whereas accumulation of trimetazidine was moderate (R_{ac} of 2.28).

### Overall

In view of the positive risk/benefit profile and the observed PK characteristics of the IMB-1018972 metabolites IMB-1028814 and trimetazidine in this single-dose and multiple-dose FIE study, further clinical development of IMB-1018972 is warranted.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A pharmaceutical composition comprising a mixture comprising:
a compound of Formula (X): 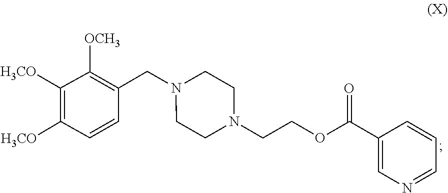 [SMILES: COc1ccc(CN2CCN(CCOC(=O)c3cccnc3)CC2)c(OC)c1OC]
and
hydroxypropyl methylcellulose (HPMC),
wherein the mixture comprises the compound of Formula (X) and HPMC in a weight ratio of from about 1:10 to about 10:1.

2. The pharmaceutical composition of claim 1, wherein the mixture comprises the compound of Formula (X) and the polymer in a weight ratio of from about 1:5 to about 5:1.

3. The pharmaceutical composition of claim 2, wherein the mixture comprises the compound of Formula (X) and the polymer in a weight ratio of from about 1:3 to about 2:1.

4. The pharmaceutical composition of claim 3, wherein the composition is a unit dosage comprising from about 10 mg to about 500 mg of the compound of Formula (X).

5. The pharmaceutical composition of claim 1, wherein the HPMC comprises a first polymeric form that has a first viscosity and a second polymeric form that has a second viscosity that is lower than the first viscosity.

6. The pharmaceutical composition of claim 5, wherein the first viscosity is at least 75,000 cP for a 2% aqueous solution of the first polymeric form of HPMC at 20° C.

7. The pharmaceutical composition of claim 5, wherein the first polymeric form comprises at least 50% by weight of the HPMC.

8. The pharmaceutical composition of claim 5, wherein the first polymeric form comprises HPMC having a degree of methoxyl substitution of from about 19% to about 24%.

9. The pharmaceutical composition of claim 5, wherein the first polymeric form comprises HPMC having a degree of hydroxypropoxyl substitution of from about 7% to about 12%.

10. The pharmaceutical composition of claim 1, wherein the composition is formulated for oral administration.

11. A method of treating a disease, disorder, condition in a subject, the method comprising providing to a subject having a disease, disorder, or condition pharmaceutical composition comprising a mixture comprising:
a compound of Formula (X): 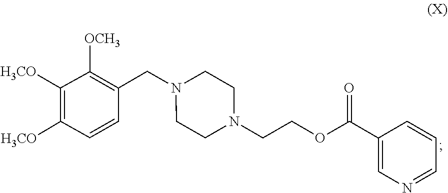 [SMILES: COc1ccc(CN2CCN(CCOC(=O)c3cccnc3)CC2)c(OC)c1OC]
and
hydroxypropyl methylcellulose (HPMC),
wherein the mixture comprises the compound of Formula (X) and HPMC in a weight ratio of from about 1:10 to about 10:1.

12. The method of claim 11, wherein the mixture comprises the compound of Formula (X) and the polymer in a weight ratio of from about 1:5 to about 5:1.

13. The method of claim 12, wherein the mixture comprises the compound of Formula (X) and the polymer in a weight ratio of from about 1:3 to about 2:1.

14. The method of claim 11, wherein the HPMC comprises a first polymeric form that has a first viscosity and a second polymeric form that has a second viscosity that is lower than the first viscosity.

15. The method of claim 14, wherein the first viscosity is at least 75,000 cP for a 2% aqueous solution of the first polymeric form of HPMC at 20° C.

16. The method of claim 14, wherein the first polymeric form comprises at least 50% by weight of the HPMC.

17. The method of claim 11, wherein the composition is provided orally to the subject.

18. The method of claim 11, wherein an interval between a first time point at which the composition is provided to the subject and a second time point at which a maximum level of a metabolite of the compound of Formula (X) is achieved in plasma of the subject is at least two hours.

19. The method of claim 11, wherein the metabolite of the compound of Formula (X) is a compound of Formula (IX):

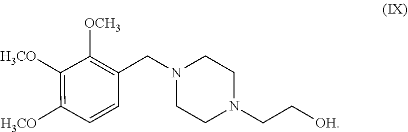 [SMILES: COc1ccc(CN2CCN(CCO)CC2)c(OC)c1OC]

20. The method of claim 11, wherein the disease, disorder, or condition is selected from the group consisting of aneurysm, angina, atherosclerosis, atherosclerosis, atherosclerosis, atherosclerosis, cardiomyopathy, cerebral vascular disease, congenital heart disease, coronary artery disease, coronary heart disease, diabetic cardiomyopathy, heart attack, heart failure, high blood pressure, ischemic heart disease, pericardial disease, peripheral arterial disease, rheumatic heart disease, stroke, transient ischemic attacks, valvular heart disease, and valvular heart disease.
